(19) United States
(12) Patent Application Publication
(10) Pub. No.: US 2026/0284084 A1
(43) Pub. Date: Sep. 24, 2026

(54) METHODS AND SYSTEMS FOR ACNE CLEARING

(71) Applicant: Face Reality, LLC, Danville, CA (US)

(72) Inventors: Laura Lee COOKSEY, Port Angeles, WA (US); Patrick Edward ROMANI, Oakland, CA (US); Nancy W. LEUNG, Oakland, CA (US); Patricia Gene CASSALIA, Oakland, CA (US)

(21) Appl. No.: 19/086,778

(22) Filed: Mar. 21, 2025

(51) Int. Cl. A61K 31/60; A61K 8/44; A61K 31/327; A61K 33/04; A61P 17/10; A61Q 19/10

(52) U.S. Cl. CPC A61K 31/60; A61K 8/44; A61K 31/327; A61K 33/04; A61P 17/10; A61Q 19/10

(57) ABSTRACT

Provided herein, in certain aspects, are method and systems for clearing acne in a subject. In some aspects, methods and systems utilize adaptive, customizable skin care regimens and acne clearing skin care formulations to improve skin quality and appearance in a subject having mild, moderate, or severe inflamed acne, or in a subject having mild, moderate, or severe noninflamed acne.

### BACKGROUND

The presence of mild, moderate, or severe acne is a common occurrence in subjects who can benefit from skin care regimens using topical formulations designed to effectively improve acne conditions. Benzoyl peroxide is used in topical formulations to treat dermatologic conditions such as acne vulgaris (commonly referred to as acne). Problems associated with topical skin care using compositions containing benzoyl peroxide may include ineffectiveness of acne clearance and/or local irritation at the site of application. There is a need for improved systems and methods of skin care regimens for effectively clearing acne.

### SUMMARY

Described herein, in some aspects, are methods for clearing inflamed or combination acne in a subject in need thereof, the methods comprising: (i) assessing an extent of acne severity in the subject; (ii) determining a skin type of the subject; (iii) assigning a customizable skin care regimen to the subject, wherein the customizable skin care regimen is initiated by the subject and comprises: a) cleansing the skin of the subject with a facial skin cleanser; b) optionally applying ice to the skin of the subject following the cleansing; c) applying a toner to the skin of the subject following the cleansing in a), or the optional application of the ice in b); d) applying a serum to the skin of the subject following the application of the toner; e) applying a topical bactericidal-based acne treatment formulation to the skin of the subject following the application of the serum, wherein the topical bactericidal-based acne treatment formulation is selected from: i] a benzoyl peroxide-based acne treatment topical formulation; ii] a salicylic acid-based acne treatment topical formulation; or iii] a sulfur-based acne treatment topical formulation; and (iv) conducting an in-clinic assessment and treatment comprising at least one facial acne peel or an acne hydrating treatment, wherein the assessment and treatment are each administered by a clinician. Described herein, in some aspects, are methods for clearing inflamed or combination acne in a subject in need thereof, the methods comprising: (i) assessing an extent of acne severity in the subject; (ii) determining a skin type of the subject; (iii) assigning a customizable skin care regimen to the subject, wherein the customizable skin care regimen is initiated by the subject and comprises: a) cleansing the skin of the subject with a facial skin cleanser; b) optionally applying ice to the skin of the subject following the cleansing; c) applying a toner to the skin of the subject following the cleansing in a), or the optional application of the ice in b); d) applying a serum to the skin of the subject following the application of the toner; e) applying a topical bactericidal-based acne treatment formulation to the skin of the subject following the application of the serum, wherein the topical bactericidal-based acne treatment formulation is selected from: i] a benzoyl peroxide-based acne treatment topical formulation; ii] a salicylic acid-based acne treatment topical formulation; or iii] a sulfur-based acne treatment topical formulation; f) applying a hydrator or a moisturizer to the skin of the subject; and g) applying a sunscreen to the skin of the subject daily in the AM; and (iv) conducting an in-clinic assessment and treatment comprising at least one facial acne peel or an acne hydrating treatment, wherein the assessment and treatment are each administered by a clinician. In some embodiments, the customizable skin care regimen comprises applying a hydrator and a moisturizer to the skin of the subject. In some embodiments, the sunscreen is a broad-spectrum sunscreen. In some embodiments, the broad-spectrum sunscreen is a mineral-based sunscreen. In some embodiments, the mineral-based sunscreen is rated to at least SPF 50. In some embodiments, the broad-spectrum sunscreen is a chemical-based sunscreen. In some embodiments, the broad-spectrum sunscreen is a non-comedogenic sunscreen. In some embodiments the non-comedogenic sunscreen is rated to at least SPF 30. In some embodiments, the non-comedogenic sunscreen is rated to at least SPF 50. In some embodiments, the in-clinic assessment and treatment begin on or around the same starting date as the customizable skin care regimen begins, and wherein the in-clinic assessment and treatment are recurrently conducted by the clinician until the subject has achieved a significant extent of clearance of inflamed acne or combination acne. In some embodiments, the in-clinic assessment and treatment are used to revise the customizable skin care regimen to achieve favorable acne clearance and improved facial skin condition in the subject. In some embodiments, the in-clinic assessment and treatment are used to revise the customizable skin care regimen to improve tolerance to skin care products selected as part of the customizable skin care regimen. In some embodiments, the subject has mild, moderate, or severe inflamed or combination acne at the time of a first in-clinic assessment. In some embodiments, the subject is assigned an initial skin care routine for twice daily use at home. In some embodiments, the initial skin care routine is customized to the skin type of the subject. In some embodiments, the skin type of the subject is (i) dry, (ii) mature, (iii) Fitzpatrick type 4, 5, or 6, (iv) normal, (v) oily and Fitzpatrick type 4, 5, or 6, (vi) oily, or (vii) oily in a subject that is pregnant or nursing (oily P/N). In some embodiments, the skin type of the subject is (i) dry, (ii) mature, or (iii) Fitzpatrick type 4, 5, or 6, and wherein the cleanser is an amino acid surfactant-based cleanser. In some embodiments, the skin type of the subject is (i) dry, and wherein the toner is a hydrating toner. In some embodiments, the skin type of the subject is (i) dry, (ii) mature, or (iii) Fitzpatrick type 4, 5, or 6, and wherein the serum is a 5% solution of an alpha hydroxy acid-based serum or an 8% solution of an alpha hydroxy acid-based serum. In some embodiments, the skin type of the subject is (i) dry, (ii) mature, or (iii) Fitzpatrick type 4, 5, or 6, and wherein the topical bactericidal-based acne treatment formulation comprises about 2.5% micronized benzoyl peroxide. In some embodiments of methods described herein, a peptide-containing moisturizer is optionally applied to the face of the subject. In some embodiments, the skin type of the subject is (iv) normal, and wherein the cleanser is a surfactant-based gel cleanser. In some embodiments, the skin type of the subject is (iv) normal, and wherein the toner is a soothing facial toner. In some embodiments, the skin type of the subject is (iv) normal, and wherein the serum is a 5% solution of an alpha hydroxy acid-based serum or an 8% solution of an alpha hydroxy acid-based serum. In some embodiments, the skin type of the subject is (iv) normal, and wherein the topical bactericidal-based acne treatment formulation comprises about 5% micronized benzoyl peroxide. In some embodiments, the skin type of the subject is (v) oily and Fitzpatrick type 4, 5, or 6, and wherein the cleanser is a surfactant-based gel cleanser. In some embodiments, the skin type of the subject is (v) oily and Fitzpatrick type 4, 5, or 6, and wherein the toner is a soothing facial toner. In some embodiments, the skin type of the subject is (v) oily and Fitzpatrick type 4, 5, or 6, wherein the serum is a 5% solution of an alpha hydroxy acid-based serum or an 8% solution of an alpha hydroxy acid-based serum. In some embodiments, the skin type of the subject is (v) oily and Fitzpatrick type 4, 5, or 6, wherein the topical bactericidal-based acne treatment formulation comprises about 2.5% micronized benzoyl peroxide. In some embodiments, the skin type of the subject is (vi) oily, and wherein the toner is a salicylic-acid and vitamin C-based toner. In some embodiments, the skin type of the subject is (vii) oily P/N, and wherein the toner is a soothing facial toner. In some embodiments, the skin type of the subject is (vi) oily or (vii) oily P/N, and wherein the cleanser is a surfactant-based gel cleanser or a surfactant-based exfoliating gel cleanser. In some embodiments, the skin type of the subject is (vi) oily or (vii) oily P/N, and wherein the serum is a 5% solution of an alpha hydroxy acid-based serum or an 8% solution of an alpha hydroxy acid-based serum. In some embodiments, the skin type of the subject is (vi) oily or (vii) oily P/N, and wherein the topical bactericidal-based acne treatment formulation comprises about 5% micronized benzoyl peroxide. In some embodiments, the applying ice to the skin of the subject is done for between about 5-90 seconds. In some embodiments, the optionally applying ice to the skin of the subject is done for at least about 5, 10, 15, 20, 25, 30, 35, 40, 45, 50, 55, 60, 70, 80, or 90 seconds. In some embodiments, the optionally applying ice to the skin of the subject is done for less than about 90, 60, 45 or 30 seconds. In some embodiments of methods described herein, an optional hydrator is applied to the skin of the subject. In some embodiments, the optional hydrator is a glycerin-based hydrating gel. In some embodiments of methods described herein, an ester-based moisturizer is optionally applied to the skin of the subject depending on an extent of facial skin dryness. In some embodiments of methods described herein, a sunscreen is applied to the face of the subject in the morning. In some embodiments of methods described herein, a non-comedogenic sunscreen is applied to the face of the subject in the morning. In some embodiments, starting from Week 3 onward in the customizable skin care regimen, sulfur-based over the counter (OTC) acne medication is applied to areas of the facial skin of the subject having inflamed acne breakouts. In some embodiments, starting from Week 7 onward in the customizable skin care regimen, the strength of the micronized benzoyl peroxide in the topical bactericidal-based acne treatment formulation used by the subject in the customizable skin care regimen is increased if the subject has not achieved sufficient acne clearance. In some embodiments, starting from Week 7 onward in the customizable skin care regimen, the strength of the micronized benzoyl peroxide in the topical bactericidal-based acne treatment formulation is increased if total inflamed acne count is the same or more than during a previous in-clinic assessment and treatment session and if the total noninflamed acne count is less, the same, or more than during the most recent in-clinic assessment and treatment session. In some embodiments, starting from Week 7 onward in the customizable skin care regimen, the strength of the serum is increased one level if total inflamed acne count is the same or more than during a previous in-clinic assessment and treatment session and the total noninflamed acne count is the same or more than during the most recent in-clinic assessment and treatment session. In some embodiments, the serum is changed to a retinaldehyde serum if the total inflamed acne count is zero and if the total noninflamed acne count is the same or more than during the most recent in-clinic assessment and treatment session. In some embodiments, no changes are made to an ongoing customizable skin care regimen if the total inflamed acne count and the total noninflamed acne count are both reduced. In some embodiments, a facial acne peel is administered by a clinician to the subject at the start of treatment in Week 1, in Week 3, in Week 5, in Week 7, in Week 9, in Week 11, in Week 13, or in Week 15, or any combination thereof if tolerated by the subject. In some embodiments, an acne hydrating treatment is administered by a clinician to the subject at the start of treatment, at Week 1, at Week 3, at Week 5, at Week 7, at Week 9, at Week 11, at Week 13, or at Week 15, or any combination thereof upon determining that an acne peel will not be well-tolerated by the subject at the time.

Described herein, in some aspects, are methods for clearing noninflamed acne in a subject in need thereof, the methods comprising: (i) assessing an extent of acne severity in the subject; (ii) determining a skin type of the subject; (iii) assigning a customizable skin care regimen to the subject, wherein the customizable skin care regimen is initiated by the subject and comprises: a) cleansing the skin of the subject with facial skin cleanser; b) applying a toner to the skin of the subject following the cleansing; c) applying a serum to the skin of the subject following the application of the toner; d) applying a topical bactericidal-based acne treatment formulation to the skin of the subject following the application of the serum, wherein the topical bactericidal-based acne treatment formulation is selected from: i] a benzoyl peroxide-based acne treatment topical formulation; ii] a salicylic acid-based acne treatment topical formulation; or iii] a sulfur-based acne treatment topical formulation; e) applying a moisturizer to the skin of the subject; and f) applying a sunscreen to the skin of the subject daily in the AM; and (iv) conducting an in-clinic assessment and treatment comprising at least one facial acne peel or an acne hydrating treatment, wherein the assessment and treatment are each administered by a clinician. In some embodiments, the in-clinic assessment and treatment begin on or around the same starting date as the customizable skin care regimen begins, and wherein the in-clinic assessment and treatment are recurrently conducted by the clinician until the subject has achieved a significant extent of clearance of inflamed acne or combination acne. In some embodiments, the in-clinic assessment and treatment are used to revise the customizable skin care regimen to achieve favorable acne clearance and improved facial skin condition in the subject. In some embodiments, the in-clinic assessment and treatment are used to revise the customizable skin care regimen to improve tolerance to skin care products selected as part of the customizable skin care regimen. In some embodiments, the subject has moderate or severe noninflamed acne, and the subject does not have inflamed acne. In some embodiments, the subject is assigned an initial skin care routine for twice daily use at home. In some embodiments, the initial skin care routine is customized to the skin type of the subject. In some embodiments, the skin type of the subject is (i) dry, (ii) mature, (iii) Fitzpatrick type 4, 5, or 6, (iv) normal, (v) oily and Fitzpatrick type 4, 5, or 6, (vi) oily, or (vii) oily in a subject that is pregnant or nursing (oily P/N). In some embodiments, the skin type of the subject is (i) dry, (ii) mature, or (iii) Fitzpatrick type 4, 5, or 6, and wherein the cleanser is an amino acid surfactant-based cleanser or a sulfate-free gel cleanser. In some embodiments, the skin type of the subject is (i) dry, (ii) mature, or (iii) Fitzpatrick type 4, 5, or 6, and wherein the toner is a humectant-based toner. In some embodiments, the skin type of the subject is (i) dry, (ii) mature, or (iii) Fitzpatrick type 4, 5, or 6, and wherein the serum is a 5% solution of an alpha hydroxy acid-based serum or an 8% solution of an alpha hydroxy acid-based serum. In some embodiments, the skin type of the subject is (i) dry, (ii) mature, or (iii) Fitzpatrick type 4, 5, or 6, and wherein the topical bactericidal-based acne treatment formulation comprises about 2.5% micronized benzoyl peroxide. In some embodiments, an optional peptide-containing moisturizer is applied to the face of the subject. In some embodiments, the skin type of the subject is (iv) normal or (v) oily and Fitzpatrick type 4, 5, or 6, and wherein the cleanser is a surfactant-based gel cleanser. In some embodiments, the skin type of the subject is (vi) oily, or (vii) oily P/N, and wherein the cleanser is a surfactant-based gel cleanser or a surfactant-based exfoliating gel cleanser. In some embodiments, the toner is a soothing facial toner. In some embodiments, the serum is a 5% solution of an alpha hydroxy acid-based serum or an 8% solution of an alpha hydroxy acid-based serum. In some embodiments, the skin type of the subject is (v) oily and Fitzpatrick type 4, 5, or 6, and wherein the topical bactericidal-based acne treatment formulation comprises about 2.5% micronized benzoyl peroxide. In some embodiments, the skin type of the subject is (iv) normal, (vi) oily, or (vii) oily P/N, and wherein the topical bactericidal-based acne treatment formulation comprises about 5% micronized benzoyl peroxide. In some embodiments, the skin type of the subject is (iv) normal, (v) oily and Fitzpatrick type 4, 5, or 6, (vi) oily, or (vii) oily P/N, and wherein the serum is a 5% solution of an alpha hydroxy acid-based serum or an 8% solution of an alpha hydroxy acid-based serum. In some embodiments, an optional hydrator is applied to the skin of the subject. In some embodiments, the optional hydrator is a glycerin-based hydrating gel. In some embodiments, an ester-based moisturizer is optionally applied to the skin of the subject depending on an extent of facial skin dryness. In some embodiments of methods described herein, a sunscreen is applied to the face of the subject in the morning. In some embodiments, from Week 7 onward after the customizable skin care regimen has been initiated by the subject, if the total noninflamed acne count increases compared to a previous assessment, application of a retinaldehyde serum is added to the customizable skin care regimen in each evening. In some embodiments, from Week 7 onward after the customizable skin care regimen has been initiated by the subject, if the total noninflamed acne count is reduced from the most recent assessment, no changes are made in the customizable skin care regimen. In some embodiments, a facial acne peel is administered by a clinician to the subject at the start of treatment in Week 1, in Week 3, in Week 5, in Week 7, in Week 9, in Week 11, in Week 13, or in Week 15, or any combination thereof if tolerated by the subject. In some embodiments, an acne hydrating treatment is administered by a clinician to the subject at the start of treatment, at Week 1, at Week 3, at Week 5, at Week 7, at Week 9, at Week 11, at Week 13, or at Week 15, or any combination thereof upon determining that an acne peel will not be well-tolerated by the subject at the time. In some embodiments, the subject achieves a significant clearance in acne compared to baseline by Week 10, by Week 12, by Week 14, or by Week 16. In some embodiments, the subject achieves a significant clearance in acne as defined by having an Investigator's Global Assessment (IGA) of acne severity score of 0 or 1, with at least a two-grade improvement from baseline. In some embodiments, the subject achieves a statistically significant clearance in acne as defined by having an IGA score of 0 or 1, with at least a two-grade improvement from baseline, by Week 14. In some embodiments, the subject achieves a statistically significant clearance in acne as defined by having an IGA score of 0 or 1, with at least a two-grade improvement from baseline, by Week 16. In some embodiments, the customizable skin care regimen comprises applying a hydrator and a moisturizer to the skin of the subject. In some embodiments, the sunscreen is a broad-spectrum sunscreen. In some embodiments, the broad-spectrum sunscreen is a mineral-based sunscreen. In some embodiments, the mineral-based sunscreen is rated to at least SPF 50. In some embodiments, the broad-spectrum sunscreen is a chemical-based sunscreen. In some embodiments, the broad-spectrum sunscreen is a non-comedogenic sunscreen. In some embodiments the non-comedogenic sunscreen is rated to at least SPF 30. In some embodiments, the non-comedogenic sunscreen is rated to at least SPF 50.

In an aspect described here in systems for clearing inflamed acne or combination acne in a subject in need thereof, the systems comprising: a facial cleanser, a toner, a serum, an OTC acne medication, a hydrator, an optional moisturizer, a sunscreen, and instructions, wherein the system is configured to perform the method steps of a customized skin care regimen comprising: i) cleansing the face of the subject at least twice daily with the facial cleanser; ii) optionally applying ice to the skin of the subject following the cleansing; iii) applying the toner to the face of the subject at least twice daily following the application of ice if ice has been applied in (ii), or following the cleansing in (i); iv) applying of a serum to the face of the subject at least twice daily following the application the toner; v) applying the OTC acne medication; vi) applying a hydrator, a moisturizer, or the hydrator and the moisturizer to the skin of the subject; and vii) applying the sunscreen to the face of the subject daily in the AM; and in-clinic treatment comprising at least one facial acne peel or an acne hydrating treatment. In some embodiments, the OTC acne medication is an advanced topical acne clearing formulation comprising micronized benzoyl peroxide. In some embodiments, the OTC acne medication is a salicylic acid-based acne treatment. In some embodiments, the OTC acne medication is a hypochlorous acid-based treatment.

In an aspect described herein, are systems for clearing noninflamed acne in a subject in need thereof, the systems comprising: a facial cleanser, a toner, a serum, an OTC acne medication, a hydrator, an optional moisturizer, a sunscreen, and instructions, wherein the system is configured to perform the method steps of a customized skin care regimen comprising: i) cleansing the face of the subject at least twice daily with the facial cleanser; ii) applying of the toner to the face of the subject at least twice daily following the cleansing; iii) applying of the serum to the face of the subject at least twice daily following the application the toner; iv) applying of the OTC acne medication; v) optionally applying the moisturizer to the face of the subject; and vi) applying the sunscreen to the face of the subject daily in the AM; and in-clinic treatment comprising at least one facial acne peel or an acne hydrating treatment. In some embodiments, the OTC acne medication is an advanced topical acne clearing formulation comprising micronized benzoyl peroxide. In some embodiments, the OTC acne medication is a salicylic acid-based acne treatment. In some embodiments, the OTC acne medication is a hypochlorous acid-based treatment.

In an aspect described herein, are systems for clearing mild noninflamed acne in a subject in need thereof, the systems comprising: a facial cleanser, a toner, a serum, a hydrator, an optional water-based moisturizer, a sunscreen, and instructions, wherein the system is configured to perform the method steps of a customized skin care regimen comprising: i) cleansing the face of the subject at least twice daily with the facial cleanser, ii) applying of the toner to the face of the subject at least twice daily following the cleansing; iii) applying of the serum to the face of the subject at least twice daily following the application the toner; iv) optional applying of the hydrator comprising a hydrating gel to the face of the subject once or twice daily following the application the serum; v) optional applying of the water-based moisturizer to the face of the subject once or twice daily following either the application the serum or the application of the hydrating gel; and vi) applying the sunscreen to the face of the subject daily in the AM; and in-clinic treatment comprising at least one facial acne peel or an acne hydrating treatment. In some embodiments, the mild noninflamed acne in the subject is improved by at least about 2 weeks, about 3 weeks, about 4 weeks, about 5 weeks, about 6 weeks, about 7 weeks, about 8 weeks, about 9 weeks, or more than about 9 weeks after the start of the customized skin care regimen. In some embodiments, the mild noninflamed acne in the subject is improved by at least about week 7 after the start of the customized skin care regimen.

Described herein, in some aspects, are methods for clearing inflamed or combination acne in a subject in need thereof, the methods comprising: (i) assessing an extent of acne severity in the subject; (ii) determining a skin type of the subject; (iii) assigning a customizable skin care regimen to the subject, wherein the customizable skin care regimen is initiated by the subject and comprises: a) cleansing the skin of the subject with a facial skin cleanser; b) optionally applying ice to the skin of the subject following the cleansing; c) applying a toner to the skin of the subject following the cleansing in a), or applying a toner to the skin of the subject following the optional application of the ice in b); d) applying a serum to the skin of the subject following the application of the toner; e) applying a topical bactericidal-based acne treatment formulation to the skin of the subject following the application of the serum, wherein the topical bactericidal-based acne treatment formulation is selected from: i] a benzoyl peroxide-based acne treatment topical formulation; ii] a salicylic acid-based acne treatment topical formulation; or iii] a sulfur-based acne treatment topical formulation; f applying a hydrator, a moisturizer, or the hydrator and the moisturizer to the skin of the subject; g) applying a non-comedogenic sunscreen to the skin of the subject daily in the AM; and (iv) conducting an in-clinic assessment and treatment comprising at least one facial acne peel or an acne hydrating treatment, wherein the assessment and treatment are each administered by a clinician. In some embodiments, the in-clinic assessment and treatment begin on or around the same starting date as the customizable skin care regimen begins, wherein the in-clinic assessment and treatment are recurrently conducted by the clinician until the subject has achieved a significant extent of clearance of inflamed acne or combination acne, and wherein the subject has mild, moderate, or severe inflamed or combination acne at the time of a first in-clinic assessment. In some embodiments, the in-clinic assessment and treatment are used to revise the customizable skin care regimen to: i) achieve favorable acne clearance and improved facial skin condition in the subject, ii) improve tolerance to skin care products selected as part of the customizable skin care regimen, or iii) achieve favorable acne clearance and improved facial skin condition in the subject and improve tolerance to skin care products selected as part of the customizable skin care regimen. In some embodiments, an optional hydrator is applied to the skin of the subject. In some embodiments, the subject is assigned an initial skin care routine for twice daily use at home, wherein the initial skin care routine is customized to the skin type of the subject, and wherein the skin type of the subject is (i) dry, (ii) mature, (iii) Fitzpatrick type 4, 5, or 6; (iv) normal, (v) oily and Fitzpatrick type 4, 5, or 6, (vi) oily, or (vii) oily in a subject that is pregnant or nursing (oily P/N). In some embodiments, the skin type of the subject is (i) dry, (ii) mature, or (iii) Fitzpatrick type 4, 5, or 6; wherein the cleanser is an amino acid surfactant-based cleanser, wherein the toner is a hydrating toner, wherein the serum is a 5% solution of an alpha hydroxy acid-based serum or an 8% solution of an alpha hydroxy acid-based serum, and wherein the topical bactericidal-based acne treatment formulation comprises about 2.5% micronized benzoyl peroxide. In some embodiments, the skin type of the subject is (iv) normal, wherein the cleanser is a surfactant-based gel cleanser, wherein the toner is a soothing facial toner, wherein the serum is a 5% solution of an alpha hydroxy acid-based serum or an 8% solution of an alpha hydroxy acid-based serum, and wherein the topical bactericidal-based acne treatment formulation comprises about 5% micronized benzoyl peroxide. In some embodiments, the skin type of the subject is (v) oily and Fitzpatrick type 4, 5, or 6; wherein the cleanser is a surfactant-based gel cleanser, wherein the toner is a soothing facial toner, wherein the serum is a 5% solution of an alpha hydroxy acid-based serum or an 8% solution of an alpha hydroxy acid-based serum, and wherein the topical bactericidal-based acne treatment formulation comprises about 2.5% micronized benzoyl peroxide. In some embodiments, the skin type of the subject is (vi) oily and wherein the toner is a salicylic-acid and vitamin C-based toner, or wherein the skin type of the subject is (vii) oily P/N and wherein the toner is a soothing facial toner; wherein the cleanser is a surfactant-based gel cleanser or a mandelic acid-based cleanser; wherein the serum is a 5% solution of an alpha hydroxy acid-based serum or an 8% solution of an alpha hydroxy acid-based serum; and wherein the topical bactericidal-based acne treatment formulation comprises about 5% micronized benzoyl peroxide. In some embodiments, from Week 7 onward after the customizable skin care regimen has been initiated by the subject, a strength of the serum applied in (iii) d) is increased if a total inflamed acne count in the subject is less compared to a total inflamed acne count at the initiation of the customizable skin care regimen and a total noninflamed acne count is the same or increased compared to a total noninflamed acne count at the initiation of the customizable skin care regimen. In some embodiments, from Week 7 onward after the customizable skin care regimen has been initiated by the subject, the serum in (iii) d) is changed to a retinaldehyde serum if a total inflamed acne count in the subject is zero and a total noninflamed acne count is the same or increased compared to a total noninflamed acne count at the initiation of the customizable skin care regimen. In some embodiments, from Week 7 onward after the customizable skin care regimen has been initiated by the subject, a strength of the topical bactericidal-based acne treatment formulation applied in (iii) e) is increased if a total inflamed acne count in the subject is the same or increases compared to a total inflamed acne count at the initiation of the customizable skin care regimen. In some embodiments, the subject achieves a significant clearance in acne as defined by having an Investigator's Global Assessment (IGA) of acne severity score of 0 or 1, with at least a two-grade improvement from baseline. In some embodiments, the non-comedogenic sunscreen is rated to at least SPF 30. In some embodiments, the non-comedogenic sunscreen is rated to at least SPF 50.

Described herein, in some aspects, are methods for clearing noninflamed acne in a subject in need thereof, the methods comprising: (i) assessing an extent of acne severity in the subject; (ii) determining a skin type of the subject; (iii) assigning a customizable skin care regimen to the subject, wherein the customizable skin care regimen is initiated by the subject and comprises: a) cleansing the skin of the subject with a facial cleanser; b) applying a toner to the skin of the subject following the cleansing; c) applying a serum to the skin of the subject following the application of the toner; d) applying a topical bactericidal-based acne treatment formulation to the skin of the subject following the application of the serum, wherein the topical bactericidal-based acne treatment formulation is selected from: i] a benzoyl peroxide-based acne treatment topical formulation; ii] a salicylic acid-based acne treatment topical formulation; or iii] a sulfur-based acne treatment topical formulation; e) applying a non-comedogenic sunscreen to the skin of the subject daily in the AM; and (iv) conducting an in-clinic assessment and treatment comprising at least one facial acne peel or an acne hydrating treatment, wherein the assessment and treatment are each administered by a clinician. In some embodiments, the in-clinic assessment and treatment begin on or around the same starting date as the customizable skin care regimen begins, wherein the in-clinic assessment and treatment are recurrently conducted by the clinician until the subject has achieved a significant extent of clearance of noninflamed acne, wherein the subject has moderate or severe noninflamed acne at the time of a first in-clinic assessment, and wherein the subject does not have inflamed acne at the time of the first in-clinic assessment. In some embodiments, the in-clinic assessment and treatment are used to revise the customizable skin care regimen to: i) achieve favorable acne clearance; ii) improve facial skin condition in the subject; iii) improve tolerance to skin care products selected as part of the customizable skin care regimen, or any combination thereof. In some embodiments, wherein the moisturizer in e) is applied, the method further comprises applying an optional hydrator to the skin of the subject to maintain skin of the subject in a healthy state of hydration so that excess oil is not produced. In some embodiments, the subject is assigned an initial skin care routine for twice daily use at home, wherein the initial skin care routine is customized to the skin type of the subject, and wherein the skin type of the subject is (1) dry, (ii) mature, (iii) Fitzpatrick type 4, 5, or 6, (iv) normal, (v) oily and Fitzpatrick type 4, 5, or 6; (vi) oily, or (vii) oily in a subject that is pregnant or nursing (oily P/N). In some embodiments of methods described herein, the skin type of the subject is: (i) dry, (ii) mature, or (iii) Fitzpatrick type 4, 5, or 6; the cleanser is an amino acid surfactant-based cleanser or a sulfate-free gel cleanser; the toner is a hydrating toner; the serum is a 5% solution of an alpha hydroxy acid-based serum or an 8% solution of an alpha hydroxy acid-based serum; and the topical bactericidal-based acne treatment formulation comprises about 2.5% micronized benzoyl peroxide. In some embodiments of methods described herein, the skin type of the subject is: (iv) normal or (v) oily and Fitzpatrick type 4, 5, or 6 and the cleanser is a surfactant-based gel cleanser; or the skin type of the subject is (vi) oily, or (vii) oily P/N and the cleanser is an surfactant-based gel cleanser or a surfactant-based exfoliating gel cleanser; the toner is a soothing facial toner; the serum is a 5% solution of an alpha hydroxy acid-based serum or an 8% solution of an alpha hydroxy acid-based serum; and the topical bactericidal-based acne treatment formulation comprises about 2.5% micronized benzoyl peroxide or about 5% micronized benzoyl peroxide. In some embodiments, from Week 7 onward after the customizable skin care regimen has been initiated by the subject, if a total noninflamed acne count in the subject increases compared to a previous assessment in the subject, application of a retinaldehyde serum to the subject is added to the customizable skin care regimen in each evening. In some embodiments, the subject achieves a significant clearance in acne as defined by having an Investigator's Global Assessment (IGA) of acne severity score of 0 or 1, with at least a two-grade improvement from baseline. In some embodiments, the non-comedogenic sunscreen is rated to at least 30 SPF. In some embodiments, the non-comedogenic sunscreen is rated to at least SPF 50.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Described herein are compositions and topical formulations to improve dermatologic conditions. The disclosure pertains to topical formulations of bactericidal-based acne treatment for use in clearing acne in a subject. In particular, the disclosure pertains to topical formulations containing benzoyl peroxide for use in clearing acne in a subject. Also described herein are adaptive systems and methods for clearing acne that use topical skin care formulations, customizable skin care regimens, and professionally administered facial treatments.

### Home Skin Care Protocols

Described herein are adaptive systems and methods for clearing acne that use topical skin care formulations, customizable skin care regimens, and professionally administered facial treatments. In some aspects, the system is for use in a subject having inflamed or combination acne. In some aspects, the method is for use in a subject having inflamed or combination acne. In some aspects, the system is for use in a subject having noninflamed acne. In some aspects, the method is for use in a subject having noninflamed acne. Home skin care protocols described herein are for use in subjects as customizable skin care regimens. In some instances, the customizable skin care regimen is conducted by the subject. In some instances, the customizable skin care regimen is conducted by the subject at-home. In some instances, the customizable skin care regimen is combined with one or more professionally administered facial treatments. In some instances, results of the customizable skin care regimen and the one or more professionally administered facial treatments are assessed and parameters are adjusted based on the effectiveness of acne clearance. In some instances, results of the customizable skin care regimen and the one or more professionally administered facial treatments are assessed and parameters are adjusted based the appearance of the subject's skin. In some instances, results of the customizable skin care regimen and the one or more professionally administered facial treatments are assessed and parameters are adjusted based on tolerability of the skin care regimen and/or facial treatments of the subject. In some instances, in-clinic assessment is used to assess results of the customizable skin care regimen. In some instances, in-clinic treatment is used to administer the one or more professionally administered facial treatments to the subject. In some instances, the customizable skin care regimen is assigned to a subject in need of cleaning inflamed or combination acne or assigned to a subject in need of clearing noninflamed acne. In some instances, the in-clinic assessment and treatment begin on or around the same starting date as the customizable skin care regimen begins, wherein the in-clinic assessment and treatment are recurrently conducted by the clinician until the subject has achieved a significant extent of clearance of inflamed acne or combination acne, and wherein the subject has mild, moderate, or severe inflamed or combination acne at the time of a first in-clinic assessment. In some instances, the in-clinic assessment and treatment are used to revise the customizable skin care regimen to: i) achieve favorable acne clearance and improved facial skin condition in the subject, ii) improve tolerance to skin care products selected as part of the customizable skin care regimen, or iii) achieve favorable acne clearance and improved facial skin condition in the subject and improve tolerance to skin care products selected as part of the customizable skin care regimen.

### Home Care Recommendations for Inflamed and Combination Acne

In some aspects described herein are adaptive systems and methods for clearing acne comprising a customizable skin care regimen for use at-home in a subject having inflamed or combination acne. In an aspect described herein are methods of clearing acne in a subject in need thereof, the methods comprising: (i) assigning a customizable skin care regimen to the subject, wherein the customizable skin care regimen comprises: (a) cleansing the skin of the subject with facial skin cleanser, (b) optionally applying ice to the skin of the subject following the cleansing, (c) applying a toner to the skin of the subject following the application of the ice if applied or applying a toner to the skin of the subject following cleansing, (d) applying a serum to the skin of the subject following the application of the toner, (e) applying a bactericidal-based acne treatment formulation to the skin of the subject following the application of the serum, (f) applying a sunscreen to the skin of the subject daily in the AM; and (ii) conducting an in-clinic assessment and treatment comprising at least one facial acne peel or an acne hydrating treatment, wherein the assessment and treatment are each administered by a clinician. In an aspect described herein are methods of clearing acne in a subject in need thereof, the methods comprising: (i) assessing an extent of acne severity in the subject; (ii) determining a skin type of the subject; (iii) assigning a customizable skin care regimen to the subject, wherein the customizable skin care regimen comprises: (a) cleansing the skin of the subject with facial skin cleanser, (b) optionally applying ice to the skin of the subject following the cleansing, (c) applying a toner to the skin of the subject following the application of the ice if applied or applying a toner to the skin of the subject following cleansing, (d) applying a serum to the skin of the subject following the application of the toner, (e) applying a bactericidal-based acne treatment formulation to the skin of the subject following the application of the serum, (f) applying a sunscreen to the skin of the subject daily in the AM; and (iv) conducting an in-clinic assessment and treatment comprising at least one facial acne peel or an acne hydrating treatment, wherein the assessment and treatment are each administered by a clinician. In an aspect described herein are adaptive systems for clearing acne in a subject in need thereof, the systems comprising: a customized skin care regimen comprising: (i) cleansing the face of the subject at least twice daily with a facial cleanser; (ii) application of ice to the face of the subject at least twice daily following the cleansing; (iii) application of a toner to the face of the subject at least twice daily following the application of ice; (iv) application of a serum to the face of the subject at least twice daily following the application the toner; (v) application of a bactericidal-based acne treatment formulation, (vi) application of a sunscreen to the skin of the subject daily in the AM; and in-clinic treatment comprising at least one facial acne peel or an acne hydrating treatment. In some embodiments, the adaptive system for clearing acne in the subject comprises skin care components used in the customized skin care regimen. In some embodiments, the skin care components comprise one or more of a cleanser, a toner, a serum, a hydrator or moisturizer, a bactericidal-based acne treatment formulation, and a broad-spectrum sunscreen. In some embodiments, the broad-spectrum sunscreen is a non-comedogenic sunscreen. In some embodiments, the skin care components comprise a cleanser, a toner, a serum, a hydrator and/or a moisturizer, a bactericidal-based acne treatment formulation, and a broad-spectrum sunscreen. In some embodiments, the adaptive system for clearing acne in the subject comprises instructions for the subject to carry out the customized skin care regimen. In some embodiments, the skin care components are adjusted based on the results of i) the effectiveness of acne clearance, ii) appearance of the subject's skin, and/or iii) tolerance to skin care products selected as part of the customizable skin care regimen to adapt the system to achieve a favorable outcome for acne clearance, aesthetics, and tolerance in the subject. In some embodiments, the bactericidal-based acne treatment formulation is an OTC acne medication. In some embodiments, the bactericidal-based acne treatment formulation is a micronized benzoyl peroxide acne treatment. In some embodiments, the bactericidal-based acne treatment formulation is an advanced topical formulation comprising between about 2.5% micronized benzoyl peroxide to about 10% micronized benzoyl peroxide. In some embodiments, the cleanser is a cream facial cleanser. In some embodiments, the cream facial cleanser is a non-foaming cleanser. In some embodiments, the cream facial cleanser is an emulsifying cleanser. In some embodiments, the cream facial cleanser is an amino acid surfactant-based cleanser. In some embodiments, the cream facial cleanser is a sodium stearoyl glutamate-based cleanser. In some embodiments, the cleanser is a gel facial cleanser. In some embodiments, the gel facial cleanser is a sulfate-free gel cleanser. In some embodiments, the gel facial cleanser is a surfactant-based gel cleanser. In some embodiments, the toner is a soothing toner. In some embodiments, the toner is a toning solution. In some embodiments, the toning solution is an exfoliating toner. In some embodiments, the toning solution is an antioxidant-based toner. In some embodiments, the toning solution is a salicylic acid-based toner. In some embodiments, the toning solution is an ascorbic acid-based toner. In some embodiments, the toning solution is a salicylic-acid and Vitamin C-based toner. In some embodiments, the serum is an alpha hydroxy acid-based serum. In some embodiments, alpha hydroxy acid-based serum is a mandelic acid-based serum. In some embodiments, the hydrator is a hydrating gel. In some embodiments, the hydrating gel is a humectant-based gel. In some embodiments, the hydrating gel is a glycerin-based hydrating gel. In some embodiments, the hydrating gel is a humectant-based gel. In some embodiments, the hydrating gel is a hydrating repair gel serum. In some embodiments, the hydrating repair gel serum is a hyaluronic acid-based serum. In some embodiments, the hydrating repair gel serum is a beta-glucan-based serum. In some embodiments, the hydrator is a water-based moisturizer. In some embodiments, the water-based moisturizer is a lightweight cream moisturizer. In some embodiments, the water-based moisturizer is an antioxidant-containing moisturizer. In some embodiments, the water-based moisturizer is an ester-based moisturizer. In some embodiments, the water-based moisturizer is an antioxidant-containing moisturizer. In some embodiments, the water-based moisturizer is an emollient cream moisturizer. In some embodiments, the water-based moisturizer is a peptide-containing moisturizer. In some embodiments, the in-clinic assessment and treatment begin on or around the same starting date as the customizable skin care regimen begins, and wherein the in-clinic assessment and treatment are recurrently conducted by the clinician until the subject has achieved a significant extent of clearance of inflamed acne or combination acne. In some embodiments, the in-clinic assessment and treatment are used to revise the customizable skin care regimen to achieve favorable acne clearance and improved facial skin condition in the subject. In some embodiments, the in-clinic assessment and treatment are used to revise the customizable skin care regimen to improve tolerance to skin care products selected as part of the customizable skin care regimen. In some embodiments, the subject has mild, moderate, or severe inflamed or combination acne at the time of a first in-clinic assessment. In some embodiments, the subject is assigned an initial skin care routine for twice daily use at home, wherein the initial skin care routine is customized to the skin type of the subject, and wherein the skin type of the subject is (i) dry, (ii) mature, (iii) Fitzpatrick type 4, 5, or 6, (iv) normal, (v) oily and Fitzpatrick type 4, 5, or 6, (vi) oily, or (vii) oily in a subject that is pregnant or nursing (oily P/N). In some embodiments, the skin type of the subject is (i) dry, (ii) mature, or (iii) Fitzpatrick type 4, 5, or 6; wherein the cleanser is an amino acid surfactant-based cleanser; and wherein the topical bactericidal-based acne treatment formulation comprises about 2.5% micronized benzoyl peroxide. In some embodiments, the skin type of the subject is (iv) normal; wherein the cleanser is a surfactant-based gel cleanser, wherein the toner is a soothing facial toner; wherein the serum is a 5% solution of an alpha hydroxy acid-based serum or an 8% solution of an alpha hydroxy acid-based serum; and wherein the topical bactericidal-based acne treatment formulation comprises about 5% micronized benzoyl peroxide. In some embodiments, the sunscreen is applied to the skin of the subject daily in the AM following the application of the 5% solution of alpha hydroxy acid-based serum or 8% solution of alpha hydroxy acid-based serum. In some embodiments, the skin type of the subject is (v) oily and Fitzpatrick type 4, 5, or 6; wherein the cleanser is a surfactant-based gel cleanser; wherein the toner is a soothing facial toner; wherein the serum is a 5% solution of an alpha hydroxy acid-based serum or an 8% solution of an alpha hydroxy acid-based serum; and wherein the topical bactericidal-based acne treatment formulation comprises about 2.5% micronized benzoyl peroxide. In some embodiments, the sunscreen is applied to the skin of the subject daily in the AM following the application of the 5% solution of alpha hydroxy acid-based serum or 8% solution of alpha hydroxy acid-based serum. In some embodiments, the skin type of the subject is (vi) oily, and wherein the toner is a salicylic-acid and vitamin C-based toner, or wherein the skin type of the subject is (vii) oily P/N, and wherein the toner is a soothing facial toner; wherein the cleanser is a surfactant-based gel cleanser or a surfactant-based exfoliating gel cleanser; wherein the serum is a 5% solution of an alpha hydroxy acid-based serum or an 8% solution of an alpha hydroxy acid-based serum; and wherein the topical bactericidal-based acne treatment formulation comprises about 5% micronized benzoyl peroxide. In some embodiments, the sunscreen is applied to the skin of the subject daily in the AM following the application of the 5% solution of alpha hydroxy acid-based serum or 8% solution of alpha hydroxy acid-based serum. In some embodiments, the subject achieves a significant clearance in acne as defined by having an Investigator's Global Assessment (IGA) of acne severity score of 0 or 1, with at least a two-grade improvement from baseline. In an aspect described here in systems for clearing inflamed acne or combination acne in a subject in need thereof, the systems comprising: a cleanser, a toner, a serum, an OTC acne medication, a hydrator, an optional moisturizer, and instructions, wherein the system is configured to perform the method steps of a customized skin care regimen comprising: i) cleansing the face of the subject at least twice daily with a facial cleanser; ii) optional application of ice to the face of the subject at least twice daily following the cleansing; iii) application of a toner to the face of the subject at least twice daily following the application of ice; iv) application of a serum to the face of the subject at least twice daily following the application the toner, v) application of an advanced topical acne clearing formulation comprising micronized benzoyl peroxide. (vi) application of a sunscreen to the skin of the subject daily in the AM; and in-clinic treatment comprising at least one facial acne peel or an acne hydrating treatment.

In some embodiments of systems and methods for clearing acne described herein, the customizable skin care regimen is for use in a subject initially assessed as having mild, moderate, or severe inflamed acne. In some embodiments, the customizable skin care regimen is for use in a subject initially assessed as having mild, moderate, or severe combination acne. In some embodiments, the customizable skin care regimen is for use in a subject initially assessed as having mild, moderate, or severe inflamed combination acne. In some instances, the subject is assessed prior to initiation of the customizable skin care regimen. In some instances, the assessment comprises a scoring of facial acne severity of the subject. In some instances, the assessment comprises a determination of skin type of the subject. In some instances, the assessment is used to determine an initial skin care routine in which the subject is instructed to initiate as an ongoing, daily at-home skin care regimen. In some instances, the subject is selected to follow an initial skin care routine wherein the selected routine is labeled as routine 1, routine 2, routine 3, routine 4, or routine 5.

### Initial Skin Care Routine 1

Following the initial assessment of the subject, initial skin care routine 1 may be started as an at-home customizable skin care regimen. In some embodiments of the system or method for clearing acne, routine 1 is appropriate for use in a subject with a dry skin type, a mature skin type, Fitzpatrick scale skin type IV, Fitzpatrick scale skin type V, or Fitzpatrick scale skin type VI, or any combination thereof. In some embodiments of the system or method for clearing acne, the initial skin care routine 1 for use in a subject with dry skin comprises use of a cleanser, optionally ice, a toner, a serum, a bactericidal-based acne treatment formulation, a hydrator, a moisturizer, a sunscreen, or optionally a spot treatment, or any combination thereof. In some embodiments of the system or method for clearing acne, the initial skin care routine 1 for use in a subject with dry skin comprises use of a cleanser, optionally ice, a toner, a serum, a bactericidal-based acne treatment formulation, a hydrator, a moisturizer, a sunscreen, and optionally a spot treatment. In some embodiments of the system or method for clearing acne, the initial skin care routine 1 for use in a subject with dry skin comprises use of a cleanser, optionally ice, a toner, a serum, an advanced topical formulation of 2.5% micronized benzoyl peroxide, a hydrator, a moisturizer, a sunscreen, and optionally a spot treatment. In some embodiments of the system or method for clearing acne, the initial skin care routine 1 for use in a subject with dry skin comprises use of a cleanser, ice, a toner, a serum, an advanced topical formulation of 2.5% micronized benzoyl peroxide, a hydrator, a moisturizer, a sunscreen, and optionally a spot treatment.

In some embodiments of the system or method for clearing acne, a cleanser, a toner, a serum, a bactericidal-based acne treatment formulation, a hydrator, a moisturizer, a sunscreen, and optionally a spot treatment is each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the toner is a hydrating toner. In some embodiments, the hydrating toner is a humectant-based toner. In some embodiments, humectant-based toner comprises sodium hyaluronate. In some embodiments, the serum is an alpha hydroxy acid-based serum. In some embodiments, the alpha hydroxy acid(s) in the alpha hydroxy acid-based serum are naturally occurring. In some embodiments, the alpha hydroxy acid(s) in the alpha hydroxy acid-based serum are synthetically derived. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a glycolic acid-based serum. In some embodiments, the alpha hydroxy acid-based serum is a lactic acid based-serum. In some embodiments, the alpha hydroxy acid-based serum is a citric acid based-serum. In some embodiments, the alpha hydroxy acid-based serum is a tartaric acid-based serum. In some embodiments, the alpha hydroxy acid-based serum is a hydroxycaproic acid-based serum. In some embodiments, the alpha hydroxy acid-based serum is a hydroxycaprylic acid-based serum. In some embodiments, the alpha hydroxy acid-based serum is a malic acid-based serum. In some embodiments, the alpha hydroxy acid-based serum is a mandelic acid-based serum. In some embodiments, the alpha hydroxy acid-based serum functions as an exfoliant. In some embodiments, the alpha hydroxy acid-based serum promotes collagen production and blood flood. In some embodiments, the alpha hydroxy acid-based serum improves discoloration from scars and age spots. In some embodiments, the alpha hydroxy acid-based serum improves the appearance of surface lines and wrinkles in the skin. In some embodiments, the alpha hydroxy acid-based serum aids in prevents acne breakouts. In some embodiments, the alpha hydroxy acid-based serum helps to loosen and remove debris, such as dead skin cells and oil in hair follicles, that could clog skin pores. In some embodiments, the alpha hydroxy acid-based serum reduces an amount of pore clog formation. In some embodiments, the alpha hydroxy acid-based serum reduces size of enlarged skin pores. In some embodiments, the alpha hydroxy acid-based serum brightens complexion of the subject. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. Salicylic acid is a beta-hydroxy acid. In some embodiments, the salicylic acid-based acne treatment formulation improves skin texture of the subject. In some embodiments, the salicylic acid-based acne treatment formulation evens sun-related skin discoloration. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is a non-comedogenic sunscreen. In some embodiments, the broad-spectrum sunscreen is at least Sun Protection Factor (SPF) 30. In some embodiments, the broad-spectrum sunscreen is at least SPF 50. In some embodiments, the sunscreen is applied daily to the face of the subject in the AM. In some embodiments, the optional spot treatment is an OTC acne spot treatment medication. In some embodiments, the OTC acne spot treatment medication is sulfur-based acne spot treatment.

In some embodiments of the system or method for clearing acne, the cleanser is a sodium stearoyl glutamate-based emulsifying cleanser. In some embodiments, the toner is a humectant-based toner. In some embodiments, the serum is a 5% mandelic acid-based serum or an 8% mandelic acid-based serum. In some embodiments, if the subject cannot tolerate mandelic acid-based serum, a salicylic acid-based serum is tried on the subject for skin tolerance. In some embodiments, the hydrator is a hydrating gel. In some embodiments, the bactericidal-based acne treatment formulation is an advanced acne medication formulation comprising about 2.5% micronized benzoyl peroxide. In some embodiments, the moisturizer is an antioxidant and peptide-containing moisturizer. In some embodiments, the sunscreen comprises a daily SPF 30 organic filter-based sunscreen. In some embodiments, the sunscreen comprises a non-comedogenic sunscreen. In some embodiments, the optional spot treatment comprises a sulfur-based acne spot treatment for use on pustules.

In some embodiments of the system or method for clearing acne, the initial skin care routine 1 for use in a subject with mature skin comprises use of a cleanser, optionally ice, a toner, a serum, a bactericidal-based acne treatment formulation, a hydrator, a moisturizer, a sunscreen, or optionally a spot treatment, or any combination thereof. In some embodiments of the system or method for clearing acne, the initial skin care routine 1 for use in a subject with mature skin comprises use of a cleanser, optionally ice, a toner, a serum, a bactericidal-based acne treatment formulation, a hydrator, a moisturizer, a sunscreen, and optionally a spot treatment. In some embodiments of the system or method for clearing acne, the initial skin care routine 1 for use in a subject with mature skin comprises use of a cleanser, optionally ice, a toner, a serum, an advanced topical formulation of about 2.5% micronized benzoyl peroxide, a hydrator, a moisturizer, a sunscreen, and optionally a spot treatment. In some embodiments of the system or method for clearing acne, the initial skin care routine 1 for use in a subject with mature skin comprises use of a cleanser, ice, a toner, a serum, an advanced topical formulation of about 2.5% micronized benzoyl peroxide, a hydrator, a moisturizer, a sunscreen, and optionally a spot treatment.

In some embodiments of the system or method for clearing acne, a cleanser, a toner, a serum, a bactericidal-based acne treatment formulation, a hydrator, a moisturizer, a sunscreen, and optionally a spot treatment is each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a hydrating toner. In some embodiments, the hydrating toner is a humectant-based toner. In some embodiments, humectant-based toner comprises sodium hyaluronate. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is a micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is a non-comedogenic sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the non-comedogenic sunscreen is at least SPF 30. In some embodiments, the non-comedogenic sunscreen is at least SPF 50. In some embodiments, the optional spot treatment is an OTC acne spot treatment medication. In some embodiments, the OTC acne spot treatment medication is sulfur-based acne spot treatment.

In some embodiments of the system or method for clearing acne, the cleanser is a sodium stearoyl glutamate-based emulsifying cleanser, a surfactant-based gel cleanser, or a sulfate-free gel cleanser. In some embodiments, the toner is a humectant-based toner. In some embodiments, the serum is a 5% mandelic acid-based serum or an 8% mandelic acid-based serum. In some embodiments, if the subject cannot tolerate mandelic acid-based serum, a salicylic acid-based serum is tried on the subject for skin tolerance. In some embodiments, the hydrator is a hydrating gel. In some embodiments, the moisturizer is an antioxidant and peptide-containing moisturizer. In some embodiments, the sunscreen comprises a daily SPF 30 organic filter-based sunscreen. In some embodiments, the sunscreen comprises a non-comedogenic sunscreen. In some embodiments, the optional spot treatment comprises a sulfur-based acne spot treatment for use on pustules.

In some embodiments of the system or method for clearing acne, the initial skin care routine 1 for use in a subject with Fitzpatrick scale skin type IV, Fitzpatrick scale skin type V, or Fitzpatrick scale skin type VI skin comprises use of a cleanser, optionally ice, a toner, a serum, a bactericidal-based acne treatment formulation, a hydrator, a moisturizer, a sunscreen, or optionally a spot treatment, or any combination thereof. In some embodiments of the system or method for clearing acne, the initial skin care routine 1 for use in a subject with Fitzpatrick scale skin type IV, Fitzpatrick scale skin type V, or Fitzpatrick scale skin type VI skin comprises use of a cleanser, optionally ice, a toner, a serum, a bactericidal-based acne treatment formulation, a hydrator, a moisturizer, a sunscreen, and optionally a spot treatment. In some embodiments of the system or method for clearing acne, the initial skin care routine 1 for use in a subject with Fitzpatrick scale skin type IV, Fitzpatrick scale skin type V, or Fitzpatrick scale skin type VI skin comprises use of a cleanser, optionally ice, a toner, a serum, an advanced topical formulation of about 2.5% micronized benzoyl peroxide, a hydrator, a moisturizer, a sunscreen, and optionally a spot treatment. In some embodiments of the system or method for clearing acne, the initial skin care routine 1 for use in a subject with Fitzpatrick scale skin type IV, Fitzpatrick scale skin type V, or Fitzpatrick scale skin type VI skin comprises use of a cleanser, ice, a toner, a serum, an advanced topical formulation of about 2.5% micronized benzoyl peroxide, a hydrator, a moisturizer, a sunscreen, and optionally a spot treatment. In some embodiments, the sunscreen is a non-comedogenic sunscreen.

In some embodiments of the system or method for clearing acne, a cleanser, a toner, a serum, a bactericidal-based acne treatment formulation, a hydrator, a moisturizer, a sunscreen, and optionally a spot treatment is each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a hydrating toner. In some embodiments, the hydrating toner is a humectant-based toner. In some embodiments, humectant-based toner comprises sodium hyaluronate. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a non-comedogenic sunscreen. In some embodiments, the broad-spectrum sunscreen is at least SPF 30. In some embodiments, the broad-spectrum sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment is an OTC acne spot treatment medication. In some embodiments, the OTC acne spot treatment medication is sulfur-based acne spot treatment.

In some embodiments of the system or method for clearing acne, the cleanser is a sodium stearoyl glutamate-based emulsifying cleanser, a surfactant-based gel cleanser, or a sulfate-free gel cleanser. In some embodiments, the toner is a humectant-based toner. In some embodiments, the serum is a 5% mandelic acid-based serum or an 8% mandelic acid-based serum. In some embodiments, if the subject cannot tolerate mandelic acid-based serum, a salicylic acid-based serum is tried on the subject for skin tolerance. In some embodiments, the hydrator is a hydrating gel. In some embodiments, the moisturizer is an antioxidant and peptide-containing moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, the spot treatment may begin at week 3 of the customizable skin care regimen. In some embodiments, for a subject with a dry skin type, a mature skin type, Fitzpatrick scale skin type IV, Fitzpatrick scale skin type V, Fitzpatrick scale skin type VI, or any combination thereof, Week 1 and Week 2 of the customizable skin care regimen comprises a daily morning (AM) and a daily evening (PM) home care protocol. In some embodiments, the Week 1 and Week 2 AM home care protocol comprises application of the cleanser, ice, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) ice, iii) the bactericidal-based acne treatment formulation according to a timed schedule, iv) cleanser, v) toner, vi) optional hydrator, and vii) moisturizer. In some embodiments, application of ice during Week 1 and Week 2 is optional. In some embodiments, for a subject with a dry skin type, a mature skin type, Fitzpatrick scale skin type IV, Fitzpatrick scale skin type V, Fitzpatrick scale skin type VI, or any combination thereof, Week 3 and Week 4 of the customizable skin care regimen comprises a daily morning (AM) and an evening (PM) home care protocol. In some embodiments, the Week 3 and Week 4 AM home care protocol comprises application of the cleanser, ice, toner, serum, optional hydrator, optional moisturizer, and sunscreen. In some embodiments, the Week 3 and Week 4 PM home care protocol comprises the following order of the application of i) the cleanser, ii) the ice, iii) the toner, iv) the optional hydrator, v) the bactericidal-based acne treatment formulation, and vi) the optional spot treatment. In some embodiments, application of ice during Week 3 and Week 4 is optional. In some embodiments, the Week 5 and Week 6 AM home care protocol comprises application of cleaner, ice, toner, serum, optional hydrator, optional moisturizer, and sunscreen. In some embodiments, the Week 5 and Week 6 PM home care protocol comprises the following order of the application of i) the cleanser, ii) the ice, iii) the toner, iv) the serum, v) the optional hydrator, vi) the bactericidal-based acne treatment formulation, and vii) the optional spot treatment. In some embodiments, application of ice during Week 5 and Week 6 is optional. In some embodiments, the Week 7 and subsequent Weeks of AM home care protocol comprises application of cleanser, ice, toner, serum, optional hydrator, optional moisturizer, and sunscreen. In some embodiments, the Week 7 and subsequent Weeks of PM home care protocol comprises the following order of the application of i) the cleanser, ii) the ice, iii) the toner, iv) the serum, v) the optional hydrator, vi) the bactericidal-based acne treatment formulation, and vii) optional spot treatment. In some embodiments, application of ice during Week 7 and during subsequent weeks is optional. In some embodiments, application of ice during Week 7 and during subsequent weeks is optional. In some embodiments of methods described herein, from Week 7 onward, after the customizable skin care regimen has been initiated by the subject, a strength of the serum applied in (iii) d) is increased if a total inflamed acne count in the subject is less compared to a total inflamed acne count at the initiation of the customizable skin care regimen and a total noninflamed acne count is the same or increased compared to a total noninflamed acne count at the initiation of the customizable skin care regimen. In some embodiments of methods described herein, from Week 7 onward after the customizable skin care regimen has been initiated by the subject, the serum in (iii) d) is changed to a retinaldehyde serum if a total inflamed acne count in the subject is zero and a total noninflamed acne count is the same or increased compared to a total noninflamed acne count at the initiation of the customizable skin care regimen. In some embodiments of methods described herein, from Week 7 onward after the customizable skin care regimen has been initiated by the subject, a strength of the topical bactericidal-based acne treatment formulation applied in (iii) e) is increased if a total inflamed acne count in the subject is the same or increases compared to a total inflamed acne count at the initiation of the customizable skin care regimen. In some embodiments, the bactericidal-based acne treatment formulation selected for use is an advanced topical formulation of about 2.5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments, the adjustments are made following an assessment by a skin care clinician. In some embodiments, during Week 7 or Week 8, if the total inflamed acne count is the same or more than at the initial assessment and the total noninflamed acne count is less, the same, or more than at the initial assessment, the subject is changed to a stronger bactericidal-based acne treatment formulation. In some embodiments, during Week 7 or Week 8, if the total inflamed acne count is the same or more than at the initial assessment and the total noninflamed acne count is less, the same, or more than at the initial assessment, the subject is changed to a stronger micronized benzoyl peroxide formulation. In some embodiments, the stronger micronized benzoyl peroxide formulation is an advanced topical formulation of about 5% micronized benzoyl peroxide. In some embodiments, during Week 7 or Week 8, if the total inflamed acne count is less than at the initial assessment and the total noninflamed acne count is the same or more than at the initial assessment, the subject is changed to increase serum strength to 1 level higher than has been applied previously. In some embodiments, during Week 7 or Week 8, if the total inflamed acne count is zero and the total noninflamed acne count is the same or more than at the initial assessment, the subject is directed to change the serum to a retinoid-based serum corrective serum. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total inflamed acne count and total noninflamed acne count is less than at the initial assessment, no changes made to the customizable skin care regimen. In some embodiments, when assessed at Week 9 or at any subsequent Week, if the subject still has inflamed acne and the total noninflamed acne count is less, same or more, any of the following updates to the customizable skin care regimen are considered for implementing that would be appropriate for the skin type of the subject: i) add OTC sulfur-based acne spot treatment as a spot treatment if the subject is not already using it, ii) add advanced topical formulation of micronized benzoyl peroxide in the morning as a spot treatment or all over, iii) add an alpha and beta-hydroxy acid based serum once a day in place of mandelic acid-based serum, iv) change to a stronger cleanser or scrub, and/or v) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) change to a stronger cleanser or scrub, and/or iv) change to a stronger toner. In some embodiments, the sunscreen is applied daily to the face of the subject in the AM. In some embodiments, the optional hydrator is applied to the skin of the subject and maintains skin of the subject in a healthy state of hydration so that excess oil is not produced. In some embodiments, the sunscreen is a non-comedogenic sunscreen. In some embodiments, the non-comedogenic sunscreen is at least SPF 30. In some embodiments, the non-comedogenic sunscreen is at least SPF 50.

### Initial Skin Care Routine 2

Following the initial assessment of the subject, initial skin care routine 2 may be started as an at-home customizable skin care regimen. In some embodiments of the system or method for clearing acne, routine 2 is appropriate for use in a subject with a normal skin type. In some embodiments of the system or method for clearing acne, the initial skin care routine 2 for use in a subject with normal skin comprises use of a cleanser, optionally ice, a toner, a serum, a bactericidal-based acne treatment formulation, a hydrator, a moisturizer, a sunscreen, or optionally a spot treatment, or any combination thereof. In some embodiments of the system or method for clearing acne, the initial skin care routine 2 for use in a subject with normal skin comprises use of a cleanser, optionally ice, a toner, a serum, a bactericidal-based acne treatment formulation, a hydrator, a moisturizer, a sunscreen, and optionally a spot treatment. In some embodiments of the system or method for clearing acne, the initial skin care routine 2 for use in a subject with normal skin comprises use of a cleanser, optionally ice, a toner, a serum, an advanced topical formulation of about 5% micronized benzoyl peroxide, a hydrator, a moisturizer, a sunscreen, and optionally a spot treatment. In some embodiments of the system or method for clearing acne, the initial skin care routine 2 for use in a subject with normal skin comprises use of a cleanser, ice, a toner, a serum, an advanced topical formulation of about 5% micronized benzoyl peroxide, a hydrator, a moisturizer, a sunscreen, and optionally a spot treatment.

In some embodiments of the system or method for clearing acne, a cleanser, a toner, a serum, a bactericidal-based acne treatment formulation, a hydrator, a moisturizer, a sunscreen, and optionally a spot treatment is each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a soothing toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional spot treatment is an OTC acne spot treatment medication. In some embodiments, the OTC acne spot treatment medication is a sulfur-based acne spot treatment.

In some embodiments of the system or method for clearing acne, the cleanser is a sulfate-free gel cleanser. In some embodiments, the toner is a soothing facial toner. In some embodiments, the serum is a 5% mandelic acid-based serum or an 8% mandelic acid-based serum. In some embodiments, if the subject cannot tolerate mandelic acid-based serum, a salicylic acid-based serum is tried on the subject for skin tolerance. In some embodiments, the bactericidal-based acne treatment formulation is an advanced acne medication formulation comprising 5% micronized benzoyl peroxide. In some embodiments, the hydrator is a hydrating gel. In some embodiments, the moisturizer is an ester-based moisturizer. In some embodiments, the sunscreen comprises a daily SPF 30 organic filter-based sunscreen. In some embodiments, the sunscreen comprises a chemical-based sunscreen. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 2, after Week 4, after Week 6, after Week 7, or after any subsequent week after Week 7 in which the in-home protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, the spot treatment may begin at week 3 of the customizable skin care regimen. In some embodiments, for a subject with a normal skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, ice, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the ice, iii) the bactericidal-based acne treatment formulation according to a timed schedule, iv) the cleanser, v) the toner, vi) the optional hydrator, and vii) the moisturizer. In some embodiments, application of ice during Week 1 and Week 2 is optional. In some embodiments, for a subject with a normal skin type, Week 3 and Week 4 of the customizable skin care regimen comprises a daily morning (AM) and an evening (PM) home care protocol. In some embodiments, the Week 3 and Week 4 AM home care protocol comprises application of the cleanser, ice, toner, serum, optional hydrator, optional moisturizer, and sunscreen. In some embodiments, the Week 3 and Week 4 PM home care protocol comprises the following order of the application of i) the cleanser, ii) the ice, iii) the toner, iv) the optional hydrator, v) the bactericidal-based acne treatment formulation, and vi) the optional spot treatment. In some embodiments, application of ice during Week 3 and Week 4 is optional. In some embodiments, the Week 5 and Week 6 AM home care protocol comprises application of cleaner, ice, toner, serum, optional hydrator, optional moisturizer, and sunscreen. In some embodiments, the Week 5 and Week 6 PM home care protocol comprises the following order of the application of i) the cleanser, ii) the ice, iii) the toner, iv) the serum, v) the optional hydrator, vi) the bactericidal-based acne treatment formulation, and vii) optional spot treatment. In some embodiments, application of ice during Week 5 and Week 6 is optional. In some embodiments, the Week 7 and subsequent Weeks of AM home care protocol comprises application of cleanser, ice, toner, serum, optional hydrator, optional moisturizer, and sunscreen. In some embodiments, the Week 7 and subsequent Weeks of PM home care protocol comprises the following order of the application of i) the cleanser, ii) the ice, iii) the toner, iv) the serum, v) the optional hydrator, vi) the bactericidal-based acne treatment formulation, and vii) the optional spot treatment. In some embodiments, application of ice during Week 7 and during subsequent weeks is optional. In some embodiments, the bactericidal-based acne treatment formulation selected for use is an advanced topical formulation of about 5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, during Week 7 or Week 8, if the total inflamed acne count is the same or more than at the initial assessment and the total noninflamed acne count is less, the same, or more than at the initial assessment, the subject is changed to stronger bactericidal-based acne treatment formulation. In some embodiments, the stronger bactericidal-based acne treatment formulation is an advanced topical formulation of about 10% micronized benzoyl peroxide. In some embodiments, during Week 7 or Week 8, if the total inflamed acne count is less than at the initial assessment and the total noninflamed acne count is the same or more than at the initial assessment, the subject is changed to increase serum strength to 1 level higher than has been applied previously. In some embodiments, during Week 7 or Week 8, if the total inflamed acne count is zero and the total noninflamed acne count is the same or more than at the initial assessment, the subject is directed to change the serum to a retinoid-based serum corrective serum. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total inflamed acne count and total noninflamed acne count is less than at the initial assessment, no changes made to the customizable skin care regimen. In some embodiments, when assessed at Week 9 or at any subsequent Week, if the subject still has inflamed acne and the total noninflamed acne count is less, same or more, any of the following updates to the customizable skin care regimen are considered for implementing that would be appropriate for the skin type of the subject: i) add OTC sulfur-based acne spot treatment as a spot treatment if the subject is not already using it, ii) add advanced topical formulation of micronized benzoyl peroxide in the morning as a spot treatment or all over, iii) add an alpha and beta-hydroxy acid based serum once a day in place of mandelic acid-based serum, iv) change to a stronger cleanser or scrub, and/or v) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) change to a stronger cleanser or scrub, and/or iv) change to a stronger toner. In some embodiments, the sunscreen is applied daily to the face of the subject in the AM. In some embodiments, the optional hydrator is applied to the skin of the subject and maintains skin of the subject in a healthy state of hydration so that excess oil is not produced. In some embodiments, the sunscreen is a non-comedogenic sunscreen. In some embodiments, the non-comedogenic sunscreen is at least SPF 30. In some embodiments, the non-comedogenic sunscreen is at least SPF 50.

### Initial Skin Care Routine 3

Following the initial assessment of the subject, initial skin care routine 3 may be started as an at-home customizable skin care regimen. In some embodiments of the system or method for clearing acne, routine 3 is appropriate for use in a subject with oily skin also determined to have Fitzpatrick scale skin type IV, Fitzpatrick scale skin type V, or Fitzpatrick scale skin type VI skin type. In some embodiments of the system or method for clearing acne, the initial skin care routine 3 is for use in a subject with oily skin also determined to have Fitzpatrick scale skin type IV, Fitzpatrick scale skin type V, or Fitzpatrick scale skin type VI skin type comprises use of a cleanser, optionally ice, a toner, a serum, a bactericidal-based acne treatment formulation, a hydrator, a moisturizer, a sunscreen, or optionally a spot treatment, or any combination thereof. In some embodiments of the system or method for clearing acne, the initial skin care routine 3 for use in a subject with oily skin also determined to have Fitzpatrick scale skin type IV, Fitzpatrick scale skin type V, or Fitzpatrick scale skin type VI skin type comprises use of a cleanser, optionally ice, a toner, a serum, a bactericidal-based acne treatment formulation, a hydrator, a moisturizer, a sunscreen, and optionally a spot treatment. In some embodiments of the system or method for clearing acne, the initial skin care routine 3 for use in a subject with oily skin also determined to have Fitzpatrick scale skin type IV, Fitzpatrick scale skin type V, or Fitzpatrick scale skin type VI skin type comprises use of a cleanser, optionally ice, a toner, a serum, an advanced topical formulation of about 2.5% micronized benzoyl peroxide, a hydrator, a moisturizer, a sunscreen, and optionally a spot treatment. In some embodiments of the system or method for clearing acne, the initial skin care routine 3 for use in a subject with oily skin also determined to have Fitzpatrick scale skin type IV, Fitzpatrick scale skin type V, or Fitzpatrick scale skin type VI skin type comprises use of a cleanser, ice, a toner, a serum, an advanced topical formulation of about 2.5% micronized benzoyl peroxide, a hydrator, a moisturizer, a sunscreen, and optionally a spot treatment.

In some embodiments of the system or method for clearing acne, a cleanser, a toner, a serum, a bactericidal-based acne treatment formulation, a hydrator, a moisturizer, a sunscreen, and optionally a spot treatment are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a soothing toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional spot treatment is an OTC acne spot treatment medication. In some embodiments, the OTC acne spot treatment medication is sulfur-based acne spot treatment.

In some embodiments of the system or method for clearing acne, the cleanser is a sulfate-free gel cleanser. In some embodiments, the toner is a soothing facial toner. In some embodiments, the serum is a 5% mandelic acid-based serum or an 8% mandelic acid-based serum. In some embodiments, if the subject cannot tolerate mandelic acid-based serum, a salicylic acid-based serum is tried on the subject for skin tolerance. In some embodiments, the bactericidal-based acne treatment formulation is an advanced acne medication formulation comprising 2.5% micronized benzoyl peroxide. In some embodiments, the hydrator is a hydrating gel. In some embodiments, the moisturizer is an ester-based moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, the spot treatment may begin at week 3 of the customizable skin care regimen. In some embodiments, for a subject with oily skin also determined to have Fitzpatrick scale skin type IV, Fitzpatrick scale skin type V, or Fitzpatrick scale skin type VI skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, ice, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the ice, iii) the bactericidal-based acne treatment formulation according to a timed schedule, iv) the cleanser, v) the toner, vi) the optional hydrator, and vii) the moisturizer. In some embodiments, application of ice during Week 1 and Week 2 is optional. In some embodiments, for a subject with oily skin also determined to have Fitzpatrick scale skin type IV, Fitzpatrick scale skin type V, or Fitzpatrick scale skin type VI skin type, Week 3 and Week 4 of the customizable skin care regimen comprises a daily morning (AM) and an evening (PM) home care protocol. In some embodiments, the Week 3 and Week 4 AM home care protocol comprises application of the cleanser, ice, toner, serum, optional hydrator, optional moisturizer, and sunscreen. In some embodiments, the Week 3 and Week 4 PM home care protocol comprises the following order of the application of i) the cleanser, ii) the ice, iii) the toner, iv) the optional hydrator, v) the bactericidal-based acne treatment formulation, and vi) the optional spot treatment. In some embodiments, application of ice during Week 3 and Week 4 is optional. In some embodiments, the Week 5 and Week 6 AM home care protocol comprises application of cleaner, ice, toner, serum, optional hydrator, optional moisturizer, and sunscreen. In some embodiments, the Week 5 and Week 6 PM home care protocol comprises the following order of the application of i) the cleanser, ii) the ice, iii) the toner, iv) the serum, v) the optional hydrator, vi) the advanced topical formulation of about 2.5% micronized benzoyl peroxide, and vii) optional spot treatment. In some embodiments, application of ice during Week 5 and Week 6 is optional. In some embodiments, the Week 7 and subsequent Weeks of AM home care protocol comprises application of cleanser, ice, toner, serum, optional hydrator, optional moisturizer, and sunscreen. In some embodiments, the Week 7 and subsequent Weeks of PM home care protocol comprises the following order of the application of i) the cleanser, ii) the ice, iii) the toner, iv) the serum, v) the optional hydrator, vi) the bactericidal-based acne treatment formulation, and vii) the optional spot treatment. In some embodiments, application of ice during Week 7 and during subsequent weeks is optional. In some embodiments, the bactericidal-based acne treatment formulation selected for use is an advanced topical formulation of about 2.5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, during Week 7 or Week 8, if the total inflamed acne count is the same or more than at the initial assessment and the total noninflamed acne count is less, the same, or more than at the initial assessment, the subject is changed to stronger bactericidal-based acne treatment formulation. In some embodiments, the stronger bactericidal-based acne treatment formulation is an advanced topical formulation of about 5% micronized benzoyl peroxide. In some embodiments, during Week 7 or Week 8, if the total inflamed acne count is less than at the initial assessment and the total noninflamed acne count is the same or more than at the initial assessment, the subject is changed to increase serum strength to 1 level higher than has been applied previously. In some embodiments, during Week 7 or Week 8, if the total inflamed acne count is zero and the total noninflamed acne count is the same or more than at the initial assessment, the subject is directed to change the serum to a retinoid-based serum corrective serum. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total inflamed acne count and total noninflamed acne count is less than at the initial assessment, no changes are made to the customizable skin care regimen. In some embodiments, when assessed at Week 9 or at any subsequent Week, if the subject still has inflamed acne and the total noninflamed acne count is less, same or more, any of the following updates to the customizable skin care regimen are considered for implementing that would be appropriate for the skin type of the subject: i) add OTC sulfur-based acne spot treatment as a spot treatment if the subject is not already using it, ii) add advanced topical formulation of micronized benzoyl peroxide in the morning as a spot treatment or all over, iii) add an alpha and beta-hydroxy acid based serum once a day in place of mandelic acid-based serum, iv) change to a stronger cleanser or scrub, and/or v) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) change to a stronger cleanser or scrub, and/or iv) change to a stronger toner. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Initial Skin Care Routine 4

Following the initial assessment of the subject, initial skin care routine 4 may be started as an at-home customizable skin care regimen. In some embodiments of the system or method for clearing acne, routine 4 is appropriate for use in a subject with oily skin irrespective of Fitzpatrick scale skin type. In some embodiments of the system or method for clearing acne, the initial skin care routine 4 for use in a subject with oily skin irrespective of Fitzpatrick scale skin type comprises use of a cleanser, optionally ice, a toner, a serum, a bactericidal-based acne treatment formulation, a hydrator, a moisturizer, a sunscreen, or optionally a spot treatment, or any combination thereof. In some embodiments of the system or method for clearing acne, the initial skin care routine 4 for use in a subject with oily skin irrespective of Fitzpatrick scale skin type comprises use of a cleanser, optionally ice, a toner, a serum, a bactericidal-based acne treatment formulation, a hydrator, a moisturizer, a sunscreen, and optionally a spot treatment. In some embodiments of the system or method for clearing acne, the initial skin care routine 4 for use in a subject with oily skin irrespective of Fitzpatrick scale skin type comprises use of a cleanser, optionally ice, a toner, a serum, an advanced topical formulation of about 5% micronized benzoyl peroxide, a hydrator, a moisturizer, a sunscreen, and optionally a spot treatment. In some embodiments of the system or method for clearing acne, the initial skin care routine 4 for use in a subject with oily skin irrespective of Fitzpatrick scale skin type comprises use of a cleanser, ice, a toner, a serum, an advanced topical formulation of about 5% micronized benzoyl peroxide, a hydrator, a moisturizer, a sunscreen, and optionally a spot treatment.

In some embodiments of the system or method for clearing acne, a cleanser, a toner, a serum, a bactericidal-based acne treatment formulation, a hydrator, a moisturizer, a sunscreen, and optionally a spot treatment are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the toner is a toning solution. In some embodiments, the toning solution is an exfoliating toner. In some embodiments, the exfoliating toner is an antioxidant-based toner. In some embodiments, the antioxidant-based toner is an ascorbic acid-based toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an inorganic-filter based sunscreen. In some embodiments, the broad-spectrum sunscreen is a mineral-based sunscreen. In some embodiments, the mineral-based sunscreen is a zinc oxide-based sunscreen. In some embodiments, the broad-spectrum sunscreen is at least SPF 28. In some embodiments, the broad-spectrum sunscreen is a mineral-based sunscreen. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional spot treatment is an OTC acne spot treatment medication. In some embodiments, the OTC acne spot treatment medication is sulfur-based acne spot treatment.

In some embodiments of the system or method for clearing acne, the cleanser is a sulfate-free gel cleanser or a mandelic acid-based cleanser. In some embodiments, the toner is an ascorbic acid-based toner. In some embodiments, the serum is a 5% mandelic acid-based serum or an 8% mandelic acid-based serum. In some embodiments, if the subject cannot tolerate mandelic acid-based serum, a salicylic acid-based serum is tried on the subject for skin tolerance. In some embodiments, the bactericidal-based acne treatment formulation is an advanced acne medication formulation comprising about 5% micronized benzoyl peroxide. In some embodiments, the hydrator is a hydrating gel. In some embodiments, the moisturizer is an ester-based moisturizer. In some embodiments, the sunscreen comprises a daily SPF 28 zinc-oxide based sunscreen. In some embodiments, the sunscreen comprises an SPF 50 mineral-sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, the spot treatment may begin at week 3 of the customizable skin care regimen. In some embodiments, for a subject with oily skin irrespective of Fitzpatrick scale skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, ice, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the ice, iii) the bactericidal-based acne treatment formulation according to a timed schedule, iv) the cleanser, v) the toner, vi) the optional hydrator, and vii) the moisturizer. In some embodiments, application of ice during Week 1 and Week 2 is optional. In some embodiments, for a subject with oily skin irrespective of Fitzpatrick scale skin type, Week 3 and Week 4 of the customizable skin care regimen comprises a daily morning (AM) and an evening (PM) home care protocol. In some embodiments, the Week 3 and Week 4 AM home care protocol comprises application of the cleanser, ice, toner, serum, optional hydrator, optional moisturizer, and sunscreen. In some embodiments, the Week 3 and Week 4 PM home care protocol comprises the following order of the application of i) the cleanser, ii) the ice, iii) the toner, iv) the optional hydrator, v) the bactericidal-based acne treatment formulation, and vi) the optional spot treatment. In some embodiments, application of ice during Week 3 and Week 4 is optional. In some embodiments, the Week 5 and Week 6 AM home care protocol comprises application of cleaner, ice, toner, serum, optional hydrator, optional moisturizer, and sunscreen. In some embodiments, the Week 5 and Week 6 PM home care protocol comprises the following order of the application of i) the cleanser, ii) the ice, iii) the toner, iv) the serum, v) the optional hydrator, vi) the bactericidal-based acne treatment formulation, and vii) optional spot treatment. In some embodiments, application of ice during Week 5 and Week 6 is optional. In some embodiments, the Week 7 and subsequent Weeks of AM home care protocol comprises application of cleanser, ice, toner, serum, optional hydrator, optional moisturizer, and sunscreen. In some embodiments, the Week 7 and subsequent Weeks of PM home care protocol comprises the following order of the application of i) the cleanser, ii) the ice, iii) the toner, iv) the serum, v) the optional hydrator, vi) the bactericidal-based acne treatment formulation, and vii) the optional spot treatment. In some embodiments, application of ice during Week 7 and during subsequent weeks is optional. In some embodiments, the bactericidal-based acne treatment formulation selected for use is an advanced topical formulation of about 5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. Home care protocols are assigned at week 1 (for week 1 & 2) and adjusted at week 3 (for weeks 3 & 4) and week 5 (for weeks 5 & 6). They may be adjusted at week 7, or after any subsequent week after Week 7 in which the home care protocols are still being conducted by the subject. In some embodiments, the adjustments are made following an assessment by a skin care clinician. In some embodiments, during Week 7 or Week 8, if the total inflamed acne count is the same or more than at the initial assessment and the total noninflamed acne count is less, the same, or more than at the initial assessment, the subject is changed to stronger bactericidal-based acne treatment formulation. In some embodiments, the stronger bactericidal-based acne treatment formulation is an advanced topical formulation of about 10% micronized benzoyl peroxide. In some embodiments, during Week 7 or Week 8, if the total inflamed acne count is less than at the initial assessment and the total noninflamed acne count is the same or more than at the initial assessment, the subject is changed to increase serum strength to 1 level higher than has been applied previously. In some embodiments, during Week 7 or Week 8, if the total inflamed acne count is zero and the total noninflamed acne count is the same or more than at the initial assessment, the subject is directed to change the serum to a retinoid-based serum corrective serum. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total inflamed acne count and total noninflamed acne count is less than at the initial assessment, no changes made to the customizable skin care regimen. In some embodiments, when assessed at Week 9 or at any subsequent Week, if the subject still has inflamed acne and the total noninflamed acne count is less, same or more, any of the following updates to the customizable skin care regimen are considered for implementing that would be appropriate for the skin type of the subject: i) add OTC sulfur-based acne spot treatment as a spot treatment if the subject is not already using it, ii) add advanced topical formulation of micronized benzoyl peroxide in the morning as a spot treatment or all over, iii) add an alpha and beta-hydroxy acid based serum once a day in place of mandelic acid-based serum, iv) change to a stronger cleanser or scrub, and/or v) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) change to a stronger cleanser or scrub, and/or iv) change to a stronger toner. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Initial Skin Care Routine 5

Following the initial assessment of the subject, initial skin care routine 5 may be started as an at-home customizable skin care regimen. In some embodiments of the system or method for clearing acne, routine 5 is appropriate for use in a subject with oily skin that is pregnant or nursing (oily P/N). In some embodiments of the system or method for clearing acne, the initial skin care routine 5 for use in a subject with oily skin that is pregnant or nursing comprises use of a cleanser, optionally ice, a toner, a serum, a bactericidal-based acne treatment formulation, a hydrator, a moisturizer, a sunscreen, or optionally a spot treatment, or any combination thereof. In some embodiments of the system or method for clearing acne, the initial skin care routine 5 for use in a subject with oily skin that is pregnant or nursing comprises use of a cleanser, optionally ice, a toner, a serum, a bactericidal-based acne treatment formulation, a hydrator, a moisturizer, a sunscreen, and optionally a spot treatment. In some embodiments of the system or method for clearing acne, the initial skin care routine 5 for use in a subject with oily skin that is pregnant or nursing comprises use of a cleanser, optionally ice, a toner, a serum, an advanced topical formulation of 5% micronized benzoyl peroxide, a hydrator, a moisturizer, a sunscreen, and optionally a spot treatment. In some embodiments of the system or method for clearing acne, the initial skin care routine 5 for use in a subject with oily skin that is pregnant or nursing comprises use of a cleanser, ice, a toner, a serum, an advanced topical formulation of 5% micronized benzoyl peroxide, a hydrator, a moisturizer, a sunscreen, and optionally a spot treatment.

In some embodiments of the system or method for clearing acne, a cleanser, a toner, a serum, a bactericidal-based acne treatment formulation, a hydrator, a moisturizer, a sunscreen, and optionally a spot treatment are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the toner is a soothing toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an inorganic-filter based sunscreen. In some embodiments, the broad-spectrum sunscreen is a mineral-based sunscreen. In some embodiments, the mineral-based sunscreen is a zinc oxide-based sunscreen. In some embodiments, the broad-spectrum sunscreen is at least SPF 28. In some embodiments, the broad-spectrum sunscreen is a mineral-based sunscreen. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional spot treatment is an OTC acne spot treatment medication. In some embodiments, the OTC acne spot treatment medication is sulfur-based acne spot treatment.

In some embodiments of the system or method for clearing acne, the cleanser is a sulfate-free gel cleanser or a mandelic acid-based cleanser. In some embodiments, the toner is a soothing toner. In some embodiments, the serum is a 5% mandelic acid-based serum or an 8% mandelic acid-based serum. In some embodiments, if the subject cannot tolerate mandelic acid-based serum, a salicylic acid-based serum is tried on the subject for skin tolerance. In some embodiments, the bactericidal-based acne treatment formulation is an advanced acne medication formulation comprising about 5% micronized benzoyl peroxide. In some embodiments, the hydrator is a hydrating gel. In some embodiments, the moisturizer is an ester-based moisturizer. In some embodiments, the sunscreen comprises a daily SPF 28 zinc-oxide based sunscreen. In some embodiments, the sunscreen comprises an SPF 50 mineral-sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. Home care protocols are assigned at week 1 (for week 1 & 2) and adjusted at week 3 (for weeks 3 & 4) and week 5 (for weeks 5 & 6). They may be adjusted at week 7, or after any subsequent week after Week 7 in which the home care protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, the spot treatment may begin at week 3 of the customizable skin care regimen. In some embodiments, for a subject with oily skin irrespective of Fitzpatrick scale skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, ice, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the ice, iii) the bactericidal-based acne treatment formulation according to a timed schedule, iv) the cleanser, v) the toner, vi) the optional hydrator, and vii) the moisturizer. In some embodiments, application of ice during Week 1 and Week 2 is optional. In some embodiments, for a subject with oily skin irrespective of Fitzpatrick scale skin type, Week 3 and Week 4 of the customizable skin care regimen comprises a daily morning (AM) and an evening (PM) home care protocol. In some embodiments, the Week 3 and Week 4 AM home care protocol comprises application of the cleanser, ice, toner, serum, optional hydrator, optional moisturizer, and sunscreen. In some embodiments, the Week 3 and Week 4 PM home care protocol comprises the following order of the application of i) the cleanser, ii) the ice, iii) the toner, iv) the optional hydrator, v) the bactericidal-based acne treatment formulation, and vi) the optional spot treatment. In some embodiments, application of ice during Week 3 and Week 4 is optional. In some embodiments, the Week 5 and Week 6 AM home care protocol comprises application of cleaner, ice, toner, serum, optional hydrator, optional moisturizer, and sunscreen. In some embodiments, the Week 5 and Week 6 PM home care protocol comprises the following order of the application of i) the cleanser, ii) the ice, iii) the toner, iv) the serum, v) the optional hydrator, vi) the bactericidal-based acne treatment formulation, and vii) optional spot treatment. In some embodiments, application of ice during Week 5 and Week 6 is optional. In some embodiments, the Week 7 and subsequent Weeks of AM home care protocol comprises application of cleanser, ice, toner, serum, optional hydrator, optional moisturizer, and sunscreen. In some embodiments, the Week 7 and subsequent Weeks of PM home care protocol comprises the following order of the application of i) the cleanser, ii) the ice, iii) the toner, iv) the serum, v) the optional hydrator, vi) the bactericidal-based acne treatment formulation, and vii) the optional spot treatment. In some embodiments, application of ice during Week 7 and during subsequent weeks is optional. In some embodiments, the bactericidal-based acne treatment formulation selected for use is an advanced topical formulation of about 5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. Home care protocols are assigned at week 1 (for week 1 & 2) and adjusted at week 3 (for weeks 3 & 4) and week 5 (for weeks 5 & 6). They may be adjusted at week 7, or after any subsequent week after Week 7 in which the home care protocols are still being conducted by the subject. In some embodiments, the adjustments are made following an assessment by a skin care clinician. In some embodiments, during Week 7 or Week 8, if the total inflamed acne count is the same or more than at the initial assessment and the total noninflamed acne count is less, the same, or more than at the initial assessment, the subject is changed to stronger bactericidal-based acne treatment formulation. In some embodiments, the stronger bactericidal-based acne treatment formulation is an advanced topical formulation of about 10% micronized benzoyl peroxide. In some embodiments, during Week 7 or Week 8, if the total inflamed acne count is less than at the initial assessment and the total noninflamed acne count is the same or more than at the initial assessment, the subject is changed to increase serum strength to 1 level higher than has been applied previously. In some embodiments, during Week 7 or Week 8, if the total inflamed acne count is zero and the total noninflamed acne count is the same or more than at the initial assessment, the subject is directed to change the serum to a retinoid-based serum corrective serum. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total inflamed acne count and total noninflamed acne count is less than at the initial assessment, no changes made to the customizable skin care regimen. In some embodiments, when assessed at Week 9 or at any subsequent Week, if the subject still has inflamed acne and the total noninflamed acne count is less, same or more, any of the following updates to the customizable skin care regimen are considered for implementing that would be appropriate for the skin type of the subject: i) add OTC sulfur-based acne spot treatment as a spot treatment if the subject is not already using it, ii) add advanced topical formulation of micronized benzoyl peroxide in the morning as a spot treatment or all over, iii) add an alpha and beta-hydroxy acid based serum once a day in place of mandelic acid-based serum, iv) change to a stronger cleanser or scrub, and/or v) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) change to a stronger cleanser or scrub, and/or iv) change to a stronger toner. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

Systems and methods described above for home care recommendations for subjects having inflamed or combination acne skin and undergoing an initial skin care routine 1-5, may be combined with in-clinic skin treatment. In some embodiments, in-clinic skin treatment comprises application of a facial acne peel. In some embodiments, in-clinic skin treatment is applied during the initial assessment. In some embodiments, in-clinic skin treatment is applied after the initial assessment. In some embodiments, in-clinic skin treatment is applied at any one of Week 1, Week 3, Week 5, Week 7, Week 9, Week 11, Week 13, Week 15, or any Week thereafter. In some embodiments, in-clinic skin treatment is applied at Week 1, Week 3, Week 5, Week 7, Week 9, Week 11, Week 13, and Week 15. In some embodiments, in-clinic skin treatment is applied at Week 1, Week 3, Week 5, Week 7, Week 9, Week 11, Week 13, Week 15, and at any subsequent weeks in which a facial acne peel is deemed appropriate by the clinician during a follow-up assessment. In some embodiments, at Week 1 in-clinic treatment, if the clinician determines severity of 0, 1, or 2 on a scale of 0-10 of tingling, stinging, and/or in a skin tolerance test with use of the glycolic-lactic toner, acid-based chemical peel 1 is performed at Week 1. In some embodiments, the acid-based chemical peel 1 is a superficial acid-based chemical peel. In some embodiments, the superficial acid-based chemical peel is a trichloroacetic acid (TCA)-based peel. In some embodiments, the superficial acid-based chemical peel is a TCA and lactic acid-based peel. In some embodiments, the superficial acid-based chemical peel is a TCA and mandelic acid-based peel. In some embodiments, the superficial acid-based chemical peel comprises between about 15% to about 40% acid, wherein the about 15% to about 40% acid comprises TCA, alpha hydroxy acids, salicylic acid, or kojic acid, or any combination thereof. In some embodiments, the superficial acid-based chemical peel comprises ones or more enzymes. In some embodiments, at Week 1 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 3 in-clinic treatment, if the clinician determines severity of 0, 1, or 2 on a scale of 0-10 of tingling, stinging, and/or burning in a skin tolerance test with use of the glycolic-lactic toner, acid-based chemical peel 1 performed at Week 3. In some embodiments, at Week 3 in-clinic treatment, if the clinician determines severity of 3 or greater on a scale of 0-10 of tingling, stinging, and/or burning skin tolerance test with use of the glycolic-lactic toner, acne hydrating treatment is performed. In some embodiments, at Week 5 in-clinic treatment, if the clinician determines severity of 0, 1, or 2 on a scale of 0-10 of tingling, stinging, and/or burning in a skin tolerance test with use of the glycolic-lactic toner, acid-based chemical peel 1 is performed at Week 5. In some embodiments, at Week 5 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week. 7 in-clinic treatment, if the clinician determines severity of 0, 1, or 2 on a scale of 0-10 of tingling, stinging, and/or burning in a skin tolerance test with use of the glycolic-lactic toner, acid-based chemical peel 2 is performed at Week 7. In some embodiments, the acid-based chemical peel 2 is a TCA-based peel. In some embodiments, the acid-based chemical peel 2 is a TCA and lactic acid-based peel. In some embodiments, the acid-based chemical peel 2 is a TCA and mandelic acid-based peel. In some embodiments, at Week 7 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 9 in-clinic treatment, if the clinician determines severity of 0, 1, or 2 on a scale of 0-10 of tingling, stinging, and/or burning in a skin tolerance test with use of the glycolic-lactic toner, acid-based chemical peel 2 is performed at Week 9. In some embodiments, at Week 9 in-clinic treatment, if the clinician determines severity of 3 or greater on a scale of 0-10 of tingling, stinging, and/or burning in a skin tolerance test with use of the glycolic-lactic toner if the clinician determines severity of 3 or greater on a scale of 0-10 of tingling, stinging, and/or burning in a skin tolerance test with use of the glycolic-lactic toner, acne hydrating treatment is performed. In some embodiments, at Week 11 in-clinic treatment, if the clinician determines severity of 0, 1, or 2 on a scale of 0-10 of tingling, stinging, and/or burning in a skin tolerance test with use of the glycolic-lactic toner, acid-based chemical peel 2 or acid-based chemical peel 3 is performed at Week 11. In some embodiments, the acid-based chemical peel 3 is a TCA-based peel. In some embodiments, the acid-based chemical peel 3 is a TCA and lactic acid-based peel. In some embodiments, the acid-based chemical peel 3 is a TCA and mandelic acid-based peel. In some embodiments, at Week 11 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 13 in-clinic treatment, if the clinician determines severity of 0, 1, or 2 on a scale of 0-10 of tingling, stinging, and/or burning in a skin tolerance test with use of the glycolic-lactic toner, acid-based chemical peel 2 or acid-based chemical peel 3 is performed at Week 13. In some embodiments, at Week 13 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 15 or any subsequent week in-clinic treatment, if the clinician determines severity of 0, 1, or 2 on a scale of 0-10 of tingling, stinging, and/or burning in a skin tolerance test with use of the glycolic-lactic toner, acid-based chemical peel 2 or acid-based chemical peel 3 is performed at Week 15 or any subsequent week in-clinic treatment. In some embodiments, at Week 15 or any subsequent week in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, the acid-based chemical peel does not comprise TCA. In some embodiments, the acid-based chemical peel that does not comprise TCA comprises between about 15% to about 40% acid, wherein the about 15% to about 40% acid comprises alpha hydroxy acids, salicylic acid, or kojic acid, or any combination thereof. In some embodiments, the acid-based chemical peel that does not comprise TCA comprises ones or more enzymes. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask.

### Home Care Recommendations for Noninflamed Only Acne

In some aspects described herein are adaptive systems and methods for clearing acne comprising a customizable skin care regimen for use at-home in a subject having noninflamed only acne. In an aspect described herein are methods of clearing acne in a subject in need thereof, the methods comprising: (i) assigning a customizable skin care regimen to the subject, wherein the customizable skin care regimen comprises: (a) cleansing the skin of the subject with facial skin cleanser, (b) applying a toner to the skin of the subject following cleansing, (c) applying a serum to the skin of the subject following the application of the toner, (d) applying a bactericidal-based acne treatment formulation to the skin of the subject following the application of the serum; and; and e) applying a sunscreen to the skin of the subject daily in the AM; and (ii) conducting an in-clinic assessment and treatment comprising at least one facial acne peel or an acne hydrating treatment, wherein the assessment and treatment are each administered by a clinician. In some embodiments, the customizable skin care regimen is for use in a subject initially assessed as having mild, moderate, or severe inflamed acne. In some embodiments, the customizable skin care regimen is for use in a subject initially assessed as having mild, moderate, or severe inflamed acne which has improved to noninflamed only acne. In some embodiments, the customizable skin care regimen is for use in a subject initially assessed as having noninflamed only acne. In some embodiments, the customizable skin care regimen is for use in a subject initially assessed as having mild, moderate, or severe noninflamed only acne. In some instances, the subject is assessed prior to initiation of the customizable skin care regimen. In some instances, the subject has noninflamed only acne from Week 1, Week 2, Week 3, Week 4, Week 5, Week 6, or Week 7, or longer than Week 7 from the start of the customizable skin care regimen. In some instances, the assessment comprises a scoring of facial acne severity of the subject. In some instances, the assessment comprises a determination of skin type of the subject. In some instances, the assessment is used to determine an initial skin care routine in which the subject is instructed to initiate as an ongoing, daily at-home skin care regimen. In some instances, the subject is selected to follow an initial skin care routine wherein the selected routine is labeled as routine 1, routine 2, routine 3, routine 4, or routine 5. In some embodiments, the subject has noninflamed only acne upon initiation and/or during the customizable skin care regimen. In an aspect described herein are methods of clearing acne in a subject in need thereof, the methods comprising: (i) assessing an extent of acne severity in the subject; (ii) determining a skin type of the subject; (iii) assigning a customizable skin care regimen to the subject, wherein the customizable skin care regimen comprises: (a) cleansing the skin of the subject with facial skin cleanser, (b) applying a toner to the skin of the subject following cleansing, (c) applying a serum to the skin of the subject following the application of the toner, (d) applying a bactericidal-based acne treatment formulation to the skin of the subject following the application of the serum; (e) applying a moisturizer to the skin of the subject; and (f) applying a sunscreen to the skin of the subject daily in the AM; and (iv) conducting an in-clinic assessment and treatment comprising at least one facial acne peel or an acne hydrating treatment, wherein the assessment and treatment are each administered by a clinician. In an aspect described herein are adaptive systems for clearing acne in a subject in need thereof, the systems comprising: a customized skin care regimen comprising: (i) cleansing the face of the subject at least twice daily with a facial cleanser; (ii) application of a toner to the face of the subject at least twice daily following the cleansing of the face of the subject; (iii) application of a serum to the face of the subject at least twice daily following the application the toner; (iv) application of a bactericidal-based acne treatment formulation; and in-clinic treatment comprising at least one facial acne peel or an acne hydrating treatment. In some embodiments, the adaptive system for clearing acne in the subject comprises skin care components used in the customized skin care regimen. In some embodiments, the skin care components comprise one or more of a cleanser, a toner, a serum, a hydrator or moisturizer, a bactericidal-based acne treatment formulation, and a broad-spectrum sunscreen. In some embodiments, the skin care components comprise a cleanser, a toner, a serum, a hydrator and/or a moisturizer, a bactericidal-based acne treatment formulation, and a broad-spectrum sunscreen. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the adaptive system for clearing acne in the subject comprises instructions for the subject to carry out the customized skin care regimen. In some embodiments, the skin care components are adjusted based on the results of i) the effectiveness of acne clearance, ii) appearance of the subject's skin, and/or iii) tolerance to skin care products selected as part of the customizable skin care regimen to adapt the system to achieve a favorable outcome for acne clearance, aesthetics, and tolerance in the subject. In some embodiments, the bactericidal-based acne treatment formulation is an OTC acne medication. In some embodiments, the bactericidal-based acne treatment formulation is a micronized benzoyl peroxide acne treatment. In some embodiments, the bactericidal-based acne treatment formulation is an advanced topical formulation comprising between about 2.5% micronized benzoyl peroxide to about 10% micronized benzoyl peroxide. In some embodiments, the in-clinic assessment and treatment are used to revise the customizable skin care regimen to: i) achieve favorable acne clearance; ii) improve facial skin condition in the subject; and iii) improve tolerance to skin care products selected as part of the customizable skin care regimen. In some embodiments, the cleanser is a cream facial cleanser. In some embodiments, the cream facial cleanser is a non-foaming cleanser. In some embodiments, the cream facial cleanser is an emulsifying cleanser. In some embodiments, the cream facial cleanser is an amino acid surfactant-based cleanser. In some embodiments, the cream facial cleanser is a sodium stearoyl glutamate-based cleanser. In some embodiments, the cleanser is a gel facial cleanser. In some embodiments, the gel facial cleanser is a sulfate-free gel cleanser. In some embodiments, the gel facial cleanser is a surfactant-based gel cleanser. In some embodiments, the cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the toner is a soothing toner. In some embodiments, the toner is a hydrating toner. In some embodiments, the hydrating toner is a humectant-based toner. In some embodiments, the serum is an alpha hydroxy acid-based serum. In some embodiments, alpha hydroxy acid-based serum is a mandelic acid-based serum. In some embodiments, the hydrator is a hydrating gel. In some embodiments, the hydrating gel is a humectant-based gel. In some embodiments, the hydrating gel is a glycerin-based hydrating gel. In some embodiments, the hydrating gel is a humectant-based gel. In some embodiments, the hydrating gel is a hydrating repair gel serum. In some embodiments, the hydrating repair gel serum is a hyaluronic acid-based serum. In some embodiments, the hydrating repair gel serum is a beta-glucan-based serum. In some embodiments, the hydrator is a water-based moisturizer. In some embodiments, the water-based moisturizer is a lightweight cream moisturizer. In some embodiments, the water-based moisturizer is an antioxidant-containing moisturizer. In some embodiments, the water-based moisturizer is an ester-based moisturizer. In some embodiments, the water-based moisturizer is an antioxidant-containing moisturizer. In some embodiments, the water-based moisturizer is an emollient cream moisturizer. In some embodiments, the water-based moisturizer is a peptide-containing moisturizer. In some embodiments, the subject has moderate or severe noninflamed acne, and the subject does not have inflamed acne. In some embodiments, the skin type of the subject is (i) dry, (ii) mature, or (iii) Fitzpatrick type 4, 5, or 6; wherein the cleanser is an amino acid surfactant-based cleanser or a sulfate-free gel cleanser; wherein the toner is a humectant-based toner; wherein the serum is a 5% solution of an alpha hydroxy acid-based serum or an 8% solution of an alpha hydroxy acid-based serum; and wherein the topical bactericidal-based acne treatment formulation comprises about 2.5% micronized benzoyl peroxide. In some embodiments, the skin type of the subject is (iv) normal or (v) oily and Fitzpatrick type 4, 5, or 6, and wherein the cleanser is a surfactant-based gel cleanser; or wherein the skin type of the subject is (vi) oily, or (vii) oily P/N, and wherein the cleanser is an surfactant-based gel cleanser or a surfactant-based exfoliating gel cleanser; wherein the toner is a soothing facial toner; wherein the serum is a 5% solution of an alpha hydroxy acid-based serum or an 8% solution of an alpha hydroxy acid-based serum; and wherein the topical bactericidal-based acne treatment formulation comprises about 2.5% micronized benzoyl peroxide or about 5% micronized benzoyl peroxide. In some embodiments of methods described herein, an optional hydrator is applied to the skin of the subject. In some embodiments of methods described herein, from Week 7 onward if the total noninflamed acne count increases compared to a previous assessment, application of a retinaldehyde serum is added to the customizable skin care regimen in each evening. In some embodiments of methods described herein, the subject achieves a significant clearance in acne as defined by having an Investigator's Global Assessment (IGA) of acne severity score of 0 or 1, with at least a two-grade improvement from baseline. In an aspect described herein, are systems for clearing noninflamed acne in a subject in need thereof, the systems comprising: a cleanser, a toner, a serum, an OTC acne medication, a hydrator, an optional moisturizer, and instructions, wherein the system is configured to perform the method steps of a customized skin care regimen comprising: i) cleansing the face of the subject at least twice daily with a facial cleanser; ii) application of a toner to the face of the subject at least twice daily following the application of ice; iii) application of a serum to the face of the subject at least twice daily following the application the toner; and iv) application of an advanced topical acne clearing formulation comprising micronized benzoyl peroxide; and in-clinic treatment comprising at least one facial acne peel or an acne hydrating treatment. In some embodiments of methods described herein, from Week 7 onward after the customizable skin care regimen has been initiated by the subject, if a total noninflamed acne count in the subject increases compared to a previous assessment in the subject, application of a retinaldehyde serum to the subject is added to the customizable skin care regimen in each evening.

### Noninflamed Initial Skin Care Routine 1

Following the initial assessment of the subject, noninflamed initial skin care routine 1 may be started as an at-home customizable skin care regimen. In some embodiments of the system or method for clearing acne, routine 1 is appropriate for use in a subject with a dry skin type, a mature skin type, Fitzpatrick scale skin type IV, Fitzpatrick scale skin type V, Fitzpatrick scale skin type VI, or any combination thereof. In some embodiments of the system or method for clearing acne, the noninflamed initial skin care routine 1 for use in a subject with dry skin comprises use of a cleanser, a toner, a serum, a bactericidal-based acne treatment formulation, a hydrator, a moisturizer, or a sunscreen, or any combination thereof. In some embodiments of the system or method for clearing acne, the noninflamed initial skin care routine 1 for use in a subject with dry skin comprises use of a cleanser, a toner, a serum, a bactericidal-based acne treatment formulation, a hydrator, a moisturizer, and a sunscreen. In some embodiments of the system or method for clearing acne, the noninflamed initial skin care routine 1 for use in a subject with dry skin comprises use of a cleanser, a toner, a serum, an advanced topical formulation of about 2.5% micronized benzoyl peroxide, a hydrator, a moisturizer, and a sunscreen. In some embodiments, the noninflamed initial skin care routine 1 for use in a subject with dry skin comprises use of a cleanser, a toner, a serum, an advanced topical formulation of about 2.5% micronized benzoyl peroxide, a hydrator, a moisturizer, and a sunscreen, and any combination thereof.

In some embodiments of the system or method of clearing acne, a cleanser, a toner, a serum, a bactericidal-based acne treatment formulation, a hydrator, a moisturizer, and a sunscreen, are each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the toner is a hydrating toner. In some embodiments, the hydrating toner is a humectant-based toner. In some embodiments, humectant-based toner comprises sodium hyaluronate. In some embodiments, the serum is an alpha hydroxy acid-based serum. In some embodiments, the alpha hydroxy acid(s) in the alpha hydroxy acid-based serum are naturally occurring. In some embodiments, the alpha hydroxy acid(s) in the alpha hydroxy acid-based serum are synthetically derived. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a glycolic acid-based serum. In some embodiments, the alpha hydroxy acid-based serum is a lactic acid based-serum. In some embodiments, the alpha hydroxy acid-based serum is a citric acid based-serum. In some embodiments, the alpha hydroxy acid-based serum is a tartaric acid-based serum. In some embodiments, the alpha hydroxy acid-based serum is a hydroxycaproic acid-based serum. In some embodiments, the alpha hydroxy acid-based serum is a hydroxycaprylic acid-based serum. In some embodiments, the alpha hydroxy acid-based serum is a malic acid-based serum. In some embodiments, the alpha hydroxy acid-based serum is a mandelic acid-based serum. In some embodiments, the alpha hydroxy acid-based serum functions as an exfoliant. In some embodiments, the alpha hydroxy acid-based serum promotes collagen production and blood flood. In some embodiments, the alpha hydroxy acid-based serum improves discoloration from scars and age spots. In some embodiments, the alpha hydroxy acid-based serum improves the appearance of surface lines and wrinkles in the skin. In some embodiments, the alpha hydroxy acid-based serum aids in prevents acne breakouts. In some embodiments, the alpha hydroxy acid-based serum helps to loosen and remove debris, such as dead skin cells and oil in hair follicles, that could clog skin pores. In some embodiments, the alpha hydroxy acid-based serum reduces an amount of pore clog formation. In some embodiments, the alpha hydroxy acid-based serum reduces size of enlarged skin pores. In some embodiments, the alpha hydroxy acid-based serum brightens complexion of the subject. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. Salicylic acid is a beta-hydroxy acid. In some embodiments, the salicylic acid-based acne treatment formulation improves skin texture of the subject. In some embodiments, the salicylic acid-based acne treatment formulation evens sun-related skin discoloration. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the sunscreen is a combination mineral and non-comedogenic sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, if the subject develops inflamed acne, an optional spot treatment is added wherein the spot treatment is an OTC acne spot treatment medication. In some embodiments, the OTC acne spot treatment medication is sulfur-based acne spot treatment. In some embodiments, the optional spot treatment comprises a sulfur-based acne spot treatment for use on pustules. In some embodiments, if the subject develops inflamed acne, the subject ceases the noninflamed initial skin care routine and starts an initial skin care routine for inflamed or combination acne according to the skin type and skin condition of the subject.

In some embodiments of the system or method of clearing acne, the cleanser is a sodium stearoyl glutamate-based emulsifying cleanser. In some embodiments, the toner is a humectant-based toner. In some embodiments, the serum is a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the hydrator is a hydrating gel. In some embodiments, the hydrating gel is a glycerin-based hydrating gel. In some embodiments, the bactericidal-based acne treatment formulation is an advanced acne medication formulation comprising about 2.5% micronized benzoyl peroxide. In some embodiments, the moisturizer is an antioxidant and peptide-containing moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment comprises a sulfur-based acne spot treatment for use on pustules that develop in the subject.

In some embodiments of the system or method for clearing acne, the noninflamed initial skin care routine 1 for use in a subject with mature skin comprises use of a cleanser, a toner, a serum, a bactericidal-based acne treatment formulation, a hydrator, a moisturizer, or a sunscreen, or any combination thereof. In some embodiments of the system or method of clearing acne, the noninflamed initial skin care routine 1 for use in a subject with mature skin comprises use of a cleanser, a toner, a serum, a bactericidal-based acne treatment formulation, a hydrator, a moisturizer, and a sunscreen. In some embodiments of the system for clearing acne, the noninflamed initial skin care routine 1 for use in a subject with mature skin comprises use of a cleanser, a toner, a serum, an advanced topical formulation of about 2.5% micronized benzoyl peroxide, a hydrator, a moisturizer, and a sunscreen. In some embodiments of the method for clearing acne, the noninflamed initial skin care routine 1 for use in a subject with mature skin comprises use of a cleanser, a toner, a serum, an advanced topical formulation of about 2.5% micronized benzoyl peroxide, a hydrator, a moisturizer, and a sunscreen.

In some embodiments of the system or method for clearing acne, a cleanser, a toner, a serum, a bactericidal-based acne treatment formulation, a hydrator, a moisturizer, and a sunscreen are each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a hydrating toner. In some embodiments, the hydrating toner is a humectant-based toner. In some embodiments, humectant-based toner comprises sodium hyaluronate. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM.

In some embodiments of the system or method for clearing acne, the noninflamed initial skin care routine 1 for use in a subject with mature skin comprises use of a cleanser, a toner, a serum, an advanced topical formulation of about 2.5% micronized benzoyl peroxide, a hydrator, a moisturizer, and a sunscreen, and any combination thereof. In some embodiments of the system or method of clearing acne, the cleanser is a sodium stearoyl glutamate-based emulsifying cleanser, a surfactant-based gel cleanser, or a sulfate-free gel cleanser. In some embodiments, the toner is a humectant-based toner. In some embodiments, the serum is a 5% mandelic acid-based serum or an 8% mandelic acid-based serum. In some embodiments, if the subject cannot tolerate mandelic acid-based serum, a 5% glycolic serum or a 10% glycolic serum is tried on the subject for skin sensitivity. In some embodiments, the moisturizer is an antioxidant and peptide-containing moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50,

In some embodiments of the system or method for clearing acne, the noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, a bactericidal-based acne treatment formulation, a hydrator, a moisturizer, or a sunscreen, or any combination thereof. In some embodiments of the system or method of clearing acne, the noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, a bactericidal-based acne treatment formulation, a hydrator, a moisturizer, and a sunscreen. In some embodiments of the system or method of clearing acne, the noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, an advanced topical formulation of about 2.5% micronized benzoyl peroxide, a hydrator, a moisturizer, and a sunscreen. In some embodiments of the system or method of clearing acne, the noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, an advanced topical formulation of about 2.5% micronized benzoyl peroxide, a hydrator, a moisturizer, and a sunscreen.

In some embodiments of the system or method of clearing acne, a cleanser, a toner, a serum, a bactericidal-based acne treatment formulation, a hydrator, a moisturizer, a sunscreen, and optionally a spot treatment if the subject develops an extent of inflamed acne, are each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a hydrating toner. In some embodiments, the hydrating toner is a humectant-based toner. In some embodiments, humectant-based toner comprises sodium hyaluronate. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM.

In some embodiments of the system or method of clearing acne, the cleanser is a sodium stearoyl glutamate-based emulsifying cleanser, a surfactant-based gel cleanser, or a sulfate-free gel cleanser. In some embodiments, the toner is a humectant-based toner. In some embodiments, the serum is a 5% mandelic acid-based serum or an 8% mandelic acid-based serum. In some embodiments, if the subject cannot tolerate mandelic acid-based serum, a 5% glycolic serum or a 10% glycolic serum is tried on the subject for skin sensitivity. In some embodiments, the hydrator is a hydrating gel. In some embodiments, the moisturizer is an antioxidant and peptide-containing moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments of the system or method for clearing acne, an optional spot treatment is applied. In some embodiments, the spot treatment is applied to one or more areas of acne pustules if the subject develops inflamed acne. In some embodiments, the spot treatment may be applied as needed to treat inflamed acne that develops in the subject after the initial assessment. In some embodiments, for a subject with a dry skin type, a mature skin type, Fitzpatrick scale skin type IV, Fitzpatrick scale skin type V, Fitzpatrick scale skin type VI, or any combination thereof, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the bactericidal-based acne treatment formulation according to a timed schedule, iii) cleanser, iv) toner, v) optional hydrator, and vi) moisturizer. In some embodiments, for a subject with a dry skin type, a mature skin type, Fitzpatrick scale skin type IV, Fitzpatrick scale skin type V, Fitzpatrick scale skin type VI, or any combination thereof, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of i) the cleanser, ii) the toner, iii) the optional hydrator, and iv) the bactericidal-based acne treatment formulation. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of i) the cleanser, ii) the toner, iii) the serum, iv) the optional hydrator, and v) the bactericidal-based acne treatment formulation. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of i) the cleanser, ii) the toner, iii) the serum, iv) the optional hydrator, and v) the bactericidal-based acne treatment formulation. In some embodiments, the bactericidal-based acne treatment formulation selected for use is an advanced topical formulation of about 2.5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinoid-based serum corrective serum is added for application in the evening to the skin care regimen. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) add advanced topical formulation of micronized benzoyl peroxide in the morning as a spot treatment or all over, ii) if the subject has been using the retinal-based serum once per day, increase application of the retinal-based serum to twice per day (once in morning and once in evening), iii) increase strength of the bactericidal-based acne treatment formulation to an advanced topical formulation of about 5% micronized benzoyl peroxide, iv) add an alpha and beta-hydroxy acid based serum once a day in place of mandelic acid-based serum, v) if the subject has been using a retinaldehyde serum twice per day, have the subject layer a 10% glycolic serum underneath the retinaldehyde serum, vi) if the subject has been using a retinaldehyde serum twice per day, change the application of the retinaldehyde serum twice per day to application of a retinol emulsion twice per day, vii) change to a stronger cleanser or scrub, and/or viii) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) increase the strength of the advanced topical formulation of micronized benzoyl peroxide to about 5% micronized benzoyl peroxide, iv) change to a stronger cleanser or scrub, and/or v) change to a stronger toner. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Noninflamed Initial Skin Care Routine 2

Following the initial assessment of the subject, noninflamed initial skin care routine 2 may be started as an at-home customizable skin care regimen. In some embodiments of the system or method for clearing acne, routine 2 is appropriate for use in a subject with a normal skin type. In some embodiments of the system or method for clearing acne, the noninflamed initial skin care routine 2 for use in a subject with normal skin comprises use of a cleanser, a toner, a serum, a bactericidal-based acne treatment formulation, a hydrator, a moisturizer, or a sunscreen, or any combination thereof. In some embodiments of the system or method for clearing acne, the noninflamed initial skin care routine 2 for use in a subject with normal skin comprises use of a cleanser, a toner, a serum, a bactericidal-based acne treatment formulation, a hydrator, a moisturizer, and a sunscreen. In some embodiments of the system for clearing acne, the noninflamed initial skin care routine 2 for use in a subject with normal skin comprises use of a cleanser, a toner, a serum, an advanced topical formulation of about 5% micronized benzoyl peroxide, a hydrator, a moisturizer, and a sunscreen. In some embodiments of the method for clearing acne, the noninflamed initial skin care routine 2 for use in a subject with normal skin comprises use of a cleanser, a toner, a serum, an advanced topical formulation of about 5% micronized benzoyl peroxide, a hydrator, a moisturizer, and a sunscreen. In some embodiments, if the subject develops inflamed acne, an optional spot treatment is added wherein the spot treatment is an OTC acne spot treatment medication. In some embodiments, the OTC acne spot treatment medication is sulfur-based acne spot treatment. In some embodiments, if the subject develops inflamed acne, the subject ceases the noninflamed initial skin care routine and starts an initial skin care routine for inflamed or combination acne according to the skin type and skin condition of the subject.

In some embodiments of the system or method for clearing acne, a cleanser, a toner, a serum, a bactericidal-based acne treatment formulation, a hydrator, a moisturizer, and a sunscreen are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a soothing toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM.

In some embodiments of the system or method for clearing acne, the cleanser is a sulfate-free gel cleanser. In some embodiments, the toner is a soothing facial toner. In some embodiments, the serum is a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the bactericidal-based acne treatment formulation is an advanced acne medication formulation comprising about 5% micronized benzoyl peroxide. In some embodiments, the hydrator is a hydrating gel. In some embodiments, the moisturizer is an ester-based moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment for a subject that develops inflamed acne comprises a sulfur-based acne spot treatment for use on pustules.

In some embodiments of the system or method for clearing acne, in home care protocols are assigned on a Week basis and may vary or be adjusted after each week, after Week 2, after Week 4, after Week 6, after Week 7, or after any subsequent week after Week 7 in which the in-home protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, for a subject with a normal skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the bactericidal-based acne treatment formulation according to a timed schedule, iii) the cleanser, iv) the toner, vi) the optional hydrator, and vii) the moisturizer. In some embodiments, for a subject with a normal skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of i) the cleanser, ii) the toner, iii) the optional hydrator, and iv) the bactericidal-based acne treatment formulation. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of i) the cleanser, ii) the toner, iii) the serum, iv) the optional hydrator, and v) the bactericidal-based acne treatment formulation. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of i) the cleanser, ii) the toner, iii) the serum, iv) the optional hydrator, and v) the bactericidal-based acne treatment formulation. In some embodiments, the bactericidal-based acne treatment formulation selected for use is an advanced topical formulation of about 5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinoid-based serum corrective serum is added for application in the evening to the skin care regimen. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) add advanced topical formulation of micronized benzoyl peroxide in the morning as a spot treatment or all over, ii) if the subject has been using the retinal-based serum once per day, increase application of the retinal-based serum to twice per day (once in morning and once in evening), iii) increase strength of the bactericidal-based acne treatment formulation to an advanced topical formulation of about 5% micronized benzoyl peroxide, iv) add an alpha and beta-hydroxy acid based serum once a day in place of mandelic acid-based serum, v) if the subject has been using a retinaldehyde serum twice per day, have the subject layer a 10% glycolic serum underneath the retinaldehyde serum, vi) if the subject has been using a retinaldehyde serum twice per day, change the application of the retinaldehyde serum twice per day to application of a retinol emulsion twice per day, vii) change to a stronger cleanser or scrub, and/or viii) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) increase the strength of the advanced topical formulation of micronized benzoyl peroxide to about 5% micronized benzoyl peroxide, iv) change to a stronger cleanser or scrub, and/or v) change to a stronger toner. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Noninflamed Initial Skin Care Routine 3

Following the initial assessment of the subject, noninflamed initial skin care routine 3 may be started as an at-home customizable skin care regimen. In some embodiments of the system or method for clearing acne, routine 3 is appropriate for use in a subject with oily skin also determined to have Fitzpatrick scale skin type IV, Fitzpatrick scale skin type V, or Fitzpatrick scale skin type VI skin type. In some embodiments of the system or method for clearing acne, the noninflamed initial skin care routine 3 is for use in a subject with oily skin also determined to have Fitzpatrick scale skin type IV, Fitzpatrick scale skin type V, or Fitzpatrick scale skin type VI skin type comprises use of a cleanser, a toner, a serum, a bactericidal-based acne treatment formulation, a hydrator, a moisturizer, or a sunscreen, or any combination thereof. In some embodiments of the system or method for clearing acne, the noninflamed initial skin care routine 3 for use in a subject with oily skin also determined to have Fitzpatrick scale skin type IV, Fitzpatrick scale skin type V, or Fitzpatrick scale skin type VI skin type comprises use of a cleanser, a toner, a serum, a bactericidal-based acne treatment formulation, a hydrator, a moisturizer, and a sunscreen. In some embodiments of the system or method for clearing acne, the noninflamed initial skin care routine 3 for use in a subject with oily skin also determined to have Fitzpatrick scale skin type IV, Fitzpatrick scale skin type V, or Fitzpatrick scale skin type VI skin type comprises use of a cleanser, a toner, a serum, an advanced topical formulation of about 2.5% micronized benzoyl peroxide, a hydrator, a moisturizer, and a sunscreen. In some embodiments of the system or method for clearing acne, the initial skin care routine 3 for use in a subject with oily skin also determined to have Fitzpatrick scale skin type IV, Fitzpatrick scale skin type V, or Fitzpatrick scale skin type VI skin type comprises use of a cleanser, a toner, a serum, an advanced topical formulation of about 2.5% micronized benzoyl peroxide, a hydrator, a moisturizer, a sunscreen, and optionally a spot treatment if the subject develops inflamed acne.

In some embodiments of the system or method for clearing acne, a cleanser, a toner, a serum, a bactericidal-based acne treatment formulation, a hydrator, a moisturizer, and a sunscreen, are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a soothing toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 2.5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, if the subject develops inflamed acne, the subject ceases the noninflamed initial skin care routine and starts an initial skin care routine for inflamed or combination acne according to the skin type and skin condition of the subject. In some embodiments, if the subject develops inflamed acne, the subject may add application of an optional spot treatment. In some embodiments, the optional spot treatment is an OTC acne spot treatment medication. In some embodiments, the OTC acne spot treatment medication is sulfur-based acne spot treatment.

In some embodiments of the system or method for clearing acne, the cleanser is a sulfate-free gel cleanser. In some embodiments, the toner is a soothing facial toner. In some embodiments, the serum is a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the bactericidal-based acne treatment formulation is an advanced acne medication formulation comprising about 2.5% micronized benzoyl peroxide. In some embodiments, the hydrator is a hydrating gel. In some embodiments, the moisturizer is an ester-based moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, for a subject with oily skin also determined to have Fitzpatrick scale skin type IV, Fitzpatrick scale skin type V, or Fitzpatrick scale skin type VI skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the bactericidal-based acne treatment formulation according to a timed schedule, iii) the cleanser, iv) the toner, v) the optional hydrator, and vi) the moisturizer. In some embodiments, for a subject with oily skin also determined to have Fitzpatrick scale skin type IV, Fitzpatrick scale skin type V, or Fitzpatrick scale skin type VI skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of i) the cleanser, ii) the toner, iii) the optional hydrator, and iv) the bactericidal-based acne treatment formulation. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of i) the cleanser, ii) the toner, iii) the serum, iv) the optional hydrator, and v) the advanced topical formulation of about 2.5% micronized benzoyl peroxide. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of i) the cleanser, ii) the toner, iii) the serum, iv) the optional hydrator, and v) the bactericidal-based acne treatment formulation. In some embodiments, the bactericidal-based acne treatment formulation selected for use is an advanced topical formulation of about 2.5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinoid-based serum corrective serum is added for application in the evening to the skin care regimen. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) add advanced topical formulation of micronized benzoyl peroxide in the morning as a spot treatment or all over, ii) if the subject has been using the retinal-based serum once per day, increase application of the retinal-based serum to twice per day (once in morning and once in evening), iii) increase strength of the bactericidal-based acne treatment formulation to an advanced topical formulation of about 5% micronized benzoyl peroxide, iv) add an alpha and beta-hydroxy acid based serum once a day in place of mandelic acid-based serum, v) if the subject has been using a retinaldehyde serum twice per day, have the subject layer a 10% glycolic serum underneath the retinaldehyde serum, vi) if the subject has been using a retinaldehyde serum twice per day, change the application of the retinaldehyde serum twice per day to application of a retinol emulsion twice per day, vii) change to a stronger cleanser or scrub, and/or viii) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) increase the strength of the advanced topical formulation of micronized benzoyl peroxide to about 5% micronized benzoyl peroxide, iv) change to a stronger cleanser or scrub, and/or v) change to a stronger toner. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Noninflamed Initial Skin Care Routine 4

Following the initial assessment of the subject, noninflamed initial skin care routine 4 may be started as an at-home customizable skin care regimen. In some embodiments of the system or method for clearing acne, routine 4 is appropriate for use in a subject with oily skin irrespective of Fitzpatrick scale skin type. In some embodiments of the system or method for clearing acne, the noninflamed initial skin care routine 4 for use in a subject with oily skin irrespective of Fitzpatrick scale skin type comprises use of a cleanser, a toner, a serum, a bactericidal-based acne treatment formulation, a hydrator, a moisturizer, or a sunscreen, or any combination thereof. In some embodiments of the system or method for clearing acne, the noninflamed initial skin care routine 4 for use in a subject with oily skin irrespective of Fitzpatrick scale skin type comprises use of a cleanser, a toner, a serum, a bactericidal-based acne treatment formulation, a hydrator, a moisturizer, and a sunscreen. In some embodiments of the system for clearing acne, the noninflamed initial skin care routine 4 for use in a subject with oily skin irrespective of Fitzpatrick scale skin type comprises use of a cleanser, a toner, a serum, an advanced topical formulation of about 5% micronized benzoyl peroxide, a hydrator, a moisturizer, and a sunscreen. In some embodiments of the method for clearing acne, the noninflamed initial skin care routine 4 for use in a subject with oily skin irrespective of Fitzpatrick scale skin type comprises use of a cleanser, a toner, a serum, an advanced topical formulation of about 5% micronized benzoyl peroxide, a hydrator, a moisturizer, and a sunscreen.

In some embodiments of the system or method for clearing acne, a cleanser, a toner, a serum, a bactericidal-based acne treatment formulation, a hydrator, a moisturizer, and a sunscreen are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the toner is a soothing toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an inorganic-filter based sunscreen. In some embodiments, the broad-spectrum sunscreen is a mineral-based sunscreen. In some embodiments, the mineral-based sunscreen is a zinc oxide-based sunscreen. In some embodiments, the broad-spectrum sunscreen is at least SPF 28. In some embodiments, the broad-spectrum sunscreen is a mineral-based sunscreen. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, if the subject develops inflamed acne, an optional spot treatment is added wherein the spot treatment is an OTC acne spot treatment medication. In some embodiments, the OTC acne spot treatment medication is sulfur-based acne spot treatment. In some embodiments, if the subject develops inflamed acne, the subject ceases the noninflamed initial skin care routine and starts an initial skin care routine for inflamed or combination acne according to the skin type and skin condition of the subject.

In some embodiments of the system or method for clearing acne, the cleanser is a sulfate-free gel cleanser or a mandelic acid-based cleanser. In some embodiments, the toner is a soothing toner. In some embodiments, the serum is a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the bactericidal-based acne treatment formulation is an advanced acne medication formulation comprising about 5% micronized benzoyl peroxide. In some embodiments, the hydrator is a hydrating gel. In some embodiments, the moisturizer is an ester-based moisturizer. In some embodiments, the sunscreen comprises a daily SPF 28 zinc-oxide based sunscreen. In some embodiments, the sunscreen comprises an SPF 50 mineral-sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, for a subject with oily skin irrespective of Fitzpatrick scale skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the bactericidal-based acne treatment formulation according to a timed schedule, iii) the cleanser, iv) the toner, v) the optional hydrator, and vi) the moisturizer. In some embodiments, for a subject with oily skin irrespective of Fitzpatrick scale skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of i) the cleanser, ii) the toner, iii) the optional hydrator, and iv) the bactericidal-based acne treatment formulation. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of i) the cleanser, ii) the toner, iii) the serum, iv) the optional hydrator, and v) the bactericidal-based acne treatment formulation. In some embodiments, an optional moisturizer is included. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of i) the cleanser, ii) the toner, iii) the serum, iv) the optional hydrator, and v) the bactericidal-based acne treatment formulation. In some embodiments, the bactericidal-based acne treatment formulation selected for use is an advanced topical formulation of about 5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinoid-based serum corrective serum is added for application in the evening to the skin care regimen. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) add advanced topical formulation of micronized benzoyl peroxide in the morning as a spot treatment or all over, ii) if the subject has been using the retinal-based serum once per day, increase application of the retinal-based serum to twice per day (once in morning and once in evening), iii) increase strength of the bactericidal-based acne treatment formulation to an advanced topical formulation of about 5% micronized benzoyl peroxide, iv) add an alpha and beta-hydroxy acid based serum once a day in place of mandelic acid-based serum, v) if the subject has been using a retinaldehyde serum twice per day, have the subject layer a 10% glycolic serum underneath the retinaldehyde serum, vi) if the subject has been using a retinaldehyde serum twice per day, change the application of the retinaldehyde serum twice per day to application of a retinol emulsion twice per day, vii) change to a stronger cleanser or scrub, and/or viii) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) increase the strength of the advanced topical formulation of micronized benzoyl peroxide to about 5% micronized benzoyl peroxide, iv) change to a stronger cleanser or scrub, and/or v) change to a stronger toner. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Noninflamed Initial Skin Care Routine 5

Following the initial assessment of the subject, noninflamed initial skin care routine 5 may be started as an at-home customizable skin care regimen. In some embodiments of the system or method for clearing acne, routine 5 is appropriate for use in a subject with oily skin that is pregnant or nursing (oily P/N). In some embodiments of the system or method for clearing acne, the noninflamed initial skin care routine 5 for use in a subject with oily skin that is pregnant or nursing comprises use of a cleanser, a toner, a serum, a bactericidal-based acne treatment formulation, a hydrator, a moisturizer, or a sunscreen, or any combination thereof. In some embodiments of the system or method for clearing acne, the noninflamed initial skin care routine 5 for use in a subject with oily skin that is pregnant or nursing comprises use of a cleanser, a toner, a serum, a bactericidal-based acne treatment formulation, a hydrator, a moisturizer, and a sunscreen. In some embodiments of the system or method for clearing acne, the noninflamed initial skin care routine 5 for use in a subject with oily skin that is pregnant or nursing comprises use of a cleanser, a toner, a serum, an advanced topical formulation of about 5% micronized benzoyl peroxide, a hydrator, a moisturizer, and a sunscreen. In some embodiments of the system or method for clearing acne, the initial skin care routine 5 for use in a subject with oily skin that is pregnant or nursing comprises use of a cleanser, a toner, a serum, an advanced topical formulation of about 5% micronized benzoyl peroxide, a hydrator, a moisturizer, and a sunscreen.

In some embodiments of the system or method for clearing acne, a cleanser, a toner, a serum, a bactericidal-based acne treatment formulation, a hydrator, a moisturizer, and a sunscreen are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the toner is a soothing toner. In some embodiments, the serum is an alpha hydroxy acid-based serum. In some embodiments, the alpha hydroxy acid-based serum is selected from a glycolic acid-based serum, a lactic acid based-serum, a citric acid based-serum, a tartaric acid-based serum, a hydroxycaproic acid-based serum, a hydroxycaprylic acid-based serum, a malic acid-based serum, or a mandelic acid-based serum. In some embodiments, the alpha hydroxy acid-based serum is a mandelic acid-based serum. In some embodiments, the bactericidal-based acne treatment formulation is an over the counter (OTC) acne medication. In some embodiments, the bactericidal-based acne treatment formulation is a benzoyl peroxide-based acne treatment formulation. In some embodiments, the benzoyl peroxide-based acne treatment formulation is micronized benzoyl peroxide acne treatment formulation. In some embodiments, the micronized benzoyl peroxide acne treatment formulation is an advanced acne medication formulation comprising about 5% micronized benzoyl peroxide. In some embodiments, the bactericidal-based acne treatment formulation is a hypochlorous-acid based treatment formulation. In some embodiments, the bactericidal-based acne treatment formulation is an anti-inflammatory based acne treatment formulation. In some embodiments, the anti-inflammatory based acne treatment formulation is a salicylic acid-based acne treatment formulation. In some embodiments, the salicylic acid-based acne treatment formulation is selected if the subject has sensitive skin. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an inorganic-filter based sunscreen. In some embodiments, the broad-spectrum sunscreen is a mineral-based sunscreen. In some embodiments, the mineral-based sunscreen is a zinc oxide-based sunscreen. In some embodiments, the broad-spectrum sunscreen is at least SPF 28. In some embodiments, the broad-spectrum sunscreen is a mineral-based sunscreen. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, if the subject develops inflamed acne, an optional spot treatment is added wherein the spot treatment is an OTC acne spot treatment medication. In some embodiments, the OTC acne spot treatment medication is sulfur-based acne spot treatment. In some embodiments, if the subject develops inflamed acne, the subject ceases the noninflamed initial skin care routine and starts an initial skin care routine for inflamed or combination acne according to the skin type and skin condition of the subject.

In some embodiments of the system or method for clearing acne, the cleanser is a sulfate-free gel cleanser or a mandelic acid-based cleanser. In some embodiments, the toner is a soothing toner. In some embodiments, the serum is a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the bactericidal-based acne treatment formulation is an advanced acne medication formulation comprising about 5% micronized benzoyl peroxide. In some embodiments, the hydrator is a hydrating gel. In some embodiments, the moisturizer is an ester-based moisturizer. In some embodiments, the sunscreen comprises a daily SPF 28 zinc-oxide based sunscreen. In some embodiments, the sunscreen comprises an SPF 50 mineral-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the optional spot treatment comprises a sulfur-based acne spot treatment for use on pustules.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments of the system or method for clearing acne, an optional spot treatment is applied. In some embodiments, the spot treatment is applied to one or more areas of acne pustules. In some embodiments, the spot treatment may be applied as needed. In some embodiments, for a subject with oily skin irrespective of Fitzpatrick scale skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of i) the cleanser, ii) the bactericidal-based acne treatment formulation according to a timed schedule, iii) the cleanser, iv) the toner, v) the optional hydrator, and vi) the moisturizer. In some embodiments, for a subject with oily skin irrespective of Fitzpatrick scale skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of i) the cleanser, ii) the toner, iv) the optional hydrator, and v) the bactericidal-based acne treatment formulation. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of i) the cleanser, ii) the toner, iii) the serum, iv) the optional hydrator, and v) the bactericidal-based acne treatment formulation. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of i) the cleanser, ii) the toner, iii) the serum, iv) the optional hydrator, and v) the bactericidal-based acne treatment formulation. In some embodiments, the bactericidal-based acne treatment formulation selected for use is an advanced topical formulation of about 5% micronized benzoyl peroxide. In some embodiments, the bactericidal-based acne treatment formulation selected for use is a hypochlorous-acid based treatment formulation. In some embodiments, the bactericidal-based acne treatment formulation selected for use is a salicylic acid-based acne treatment formulation. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinoid-based serum corrective serum is added for application in the evening to the skin care regimen. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) if the subject has been using the retinal-based serum once per day, increase application of the retinal-based serum to twice per day (once in morning and once in evening), ii) increase strength of the bactericidal-based acne treatment formulation to an advanced topical formulation of about 5% micronized benzoyl peroxide, iii) add an alpha and beta-hydroxy acid based serum once a day in place of mandelic acid-based serum, iv) if the subject has been using a retinaldehyde serum twice per day, have the subject layer a 10% glycolic serum underneath the retinaldehyde serum, v) if the subject has been using a retinaldehyde serum twice per day, change the application of the retinaldehyde serum twice per day to application of a retinol emulsion twice per day, vi) change to a stronger cleanser or scrub, and/or vii) change to a stronger toner. In some embodiments, the alpha and beta-hydroxy acid-based serum is a salicylic acid-based serum. In some embodiments, when assessed at Week 9 or at any subsequent Week, if the subject has no inflamed acne and they still have noninflamed acne, consider implementing any of the following that would be appropriate for the skin type: i) add a retinoid-based corrective serum in the evening in place of their current serum, ii) if the subject has already been using a retinaldehyde serum as the retinoid-based corrective serum and their noninflamed acne has not shown improvement, move the subject to a retinol emulsion as the retinoid-based serum in the evening (PM), iii) increase the strength of the advanced topical formulation of micronized benzoyl peroxide to about 5% micronized benzoyl peroxide, iv) change to a stronger cleanser or scrub, and/or v) change to a stronger toner. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

Systems and methods described above for home care recommendations for subjects having noninflamed acne or combination acne skin and undergoing an initial skin care routine 1-5, may be combined with in-clinic skin treatment. In some embodiments, in-clinic skin treatment comprises application of a facial acne peel. In some embodiments, in-clinic skin treatment is applied during the initial assessment. In some embodiments, in-clinic skin treatment is applied after the initial assessment. In some embodiments, in-clinic skin treatment is applied at any one of Week 1, Week 3, Week 5, Week 7, Week 9, Week 11, Week 13, Week 15, or any Week thereafter. In some embodiments, in-clinic skin treatment is applied at Week 1, Week 3, Week 5, Week 7, Week 9, Week 11, Week 13, and Week 15. In some embodiments, in-clinic skin treatment is applied at Week 1, Week 3, Week 5, Week 7, Week 9, Week 11, Week 13, Week 15, and at any subsequent weeks in which a facial acne peel is deemed appropriate by the clinician during a follow-up assessment. In some embodiments, at Week 1 in-clinic treatment, if the clinician determines severity of 0, 1, or 2 on a scale of 0-10 of tingling, stinging, and/or burning in a skin tolerance test with use of the exfoliating toner, acid-based chemical peel 1 is performed at Week 1. In some embodiments, the acid-based chemical peel 1 is a superficial acid-based chemical peel 1. In some embodiments, the superficial acid-based chemical peel 1 is a trichloroacetic acid (TCA)-based peel. In some embodiments, the superficial acid-based chemical peel 1 is a TCA and lactic acid-based peel. In some embodiments, the superficial acid-based chemical peel 1 is a TCA and mandelic acid-based peel. In some embodiments, the superficial acid-based chemical peel 1 comprises between about 15% to about 40% acid, wherein the about 15% to about 40% acid comprises TCA, alpha hydroxy acids, salicylic acid, or kojic acid, or any combination thereof. In some embodiments, the superficial acid-based chemical peel comprises ones or more enzymes. In some embodiments, at Week 1 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 3 in-clinic treatment, if the clinician determines severity of 0, 1, or 2 on a scale of 0-10 of tingling, stinging, and/or burning in a skin tolerance test with use of the exfoliating toner, acid-based chemical peel 1 is performed at Week 3. In some embodiments, at Week 3 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 5 in-clinic treatment, if the clinician determines severity of 0, 1, or 2 on a scale of 0-10 of tingling, stinging, and/or burning in a skin tolerance test with use of the exfoliating toner, acid-based chemical peel 1 is performed at Week 5. In some embodiments, at Week 5 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 7 in-clinic treatment, if the clinician determines severity of 0, 1, or 2 on a scale of 0-10 of tingling, stinging, and/or burning in a skin tolerance test with use of the exfoliating toner, acid-based chemical peel 2 is performed at Week 7. In some embodiments, the acid-based chemical peel 2 is a TCA-based peel. In some embodiments, the acid-based chemical peel 2 is a TCA and lactic acid-based peel. In some embodiments, the acid-based chemical peel 2 is a TCA and mandelic acid-based peel. In some embodiments, at Week 7 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 9 in-clinic treatment, if the clinician determines severity of 0, 1, or 2 on a scale of 0-10 of tingling, stinging, and/or burning in a skin tolerance test with use of the exfoliating toner, acid-based chemical peel 2 is performed at Week 9. In some embodiments, at Week 9 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 11 in-clinic treatment, if the clinician determines severity of 0, 1, or 2 on a scale of 0-10 of tingling, stinging, and/or burning in a skin tolerance test with use of the exfoliating toner, acid-based chemical peel 2 or acid-based chemical peel 3 is performed at Week 11. In some embodiments, the acid-based chemical peel 3 is a TCA-based peel. In some embodiments, the acid-based chemical peel 3 is a TCA and lactic acid-based peel. In some embodiments, the acid-based chemical peel 3 is a TCA and mandelic acid-based peel. In some embodiments, at Week 11 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 13 in-clinic treatment, if the clinician determines severity of 0, 1, or 2 on a scale of 0-10 of tingling, stinging, and/or burning in a skin tolerance test with use of the exfoliating toner, acid-based chemical peel 2 or acid-based chemical peel 3 is performed at Week 13. In some embodiments, at Week 13 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 15 or any subsequent week in-clinic treatment, if the clinician determines severity of 0, 1, or 2 on a scale of 0-10 of tingling, stinging, and/or burning in a skin tolerance test with use of the exfoliating toner, acid-based chemical peel 2 or acid-based chemical peel 3 is performed at Week 15 or any subsequent week in-clinic treatment. In some embodiments, at Week 15 or any subsequent week in-clinic treatment, if the clinician determines severity of 3 or greater on a scale of 0-10 of tingling, stinging, and/or burning in a skin tolerance test with use of the exfoliating toner, acne hydrating treatment is performed.

### Adaptive Systems and Methods for Clearing Mild Non-Inflamed Acne

In some aspects described herein are adaptive systems and methods for clearing acne comprising a customizable skin care regimen for use at-home in a subject having mild noninflamed acne. In an aspect described herein are methods of clearing mild noninflamed acne in a subject in need thereof, the methods comprising: (i) assigning a customizable skin care regimen to the subject, wherein the customizable skin care regimen comprises: (a) cleansing the skin of the subject with facial skin cleanser, (b) applying a toner to the skin of the subject following cleansing, (c) applying a serum to the skin of the subject following the application of the toner, and (ii) conducting an in-clinic assessment and treatment comprising at least one facial acne peel or an acne hydrating treatment, wherein the assessment and treatment are each administered by a clinician. In some embodiments, a sunscreen is applied daily to the face of the subject in the AM. In some embodiments, the customizable skin care regimen is for use in a subject initially assessed as having mild noninflamed acne. In some embodiments, the customizable skin care regimen is for use in a subject initially assessed as having mild, moderate, or severe inflamed acne, or moderate to severe noninflamed acne which has improved to mild noninflamed only acne. In some embodiments, the customizable skin care regimen is for use in a subject initially assessed as having mild noninflamed only acne. In some instances, the subject is assessed prior to initiation of the customizable skin care regimen. In some instances, the subject has mild noninflamed only acne from Week 1, Week 2, Week 3, Week 4, Week 5, Week 6, or Week 7, or longer than Week 7 from the start of the customizable skin care regimen. In some instances, the assessment comprises a scoring of facial acne severity of the subject. In some instances, the assessment comprises a determination of skin type of the subject. In some instances, the assessment is used to determine an initial skin care routine in which the subject is instructed to initiate as an ongoing, daily at-home skin care regimen. In some instances, the subject is selected to follow an initial skin care routine wherein the selected routine is labeled as routine 1, routine 2, routine 3, routine 4, or routine 5. In some embodiments, the subject has mild noninflamed only acne upon initiation and/or during the customizable skin care regimen. In an aspect described herein are methods of clearing mild noninflamed acne in a subject in need thereof, the methods comprising: (i) assessing an extent of acne severity in the subject; (ii) determining a skin type of the subject; (iii) assigning a customizable skin care regimen to the subject, wherein the customizable skin care regimen comprises: (a) cleansing the skin of the subject with facial skin cleanser, (b) applying a toner to the skin of the subject following cleansing, (c) applying a serum to the skin of the subject following the application of the toner; and (iv) conducting an in-clinic assessment and treatment comprising at least one facial acne peel or an acne hydrating treatment, wherein the assessment and treatment are each administered by a clinician. In an aspect described herein are adaptive systems for clearing mild noninflamed acne in a subject in need thereof, the systems comprising: a customized skin care regimen comprising: (i) cleansing the face of the subject at least twice daily with a facial cleanser; (ii) application of a toner to the face of the subject at least twice daily following the cleansing of the face of the subject; (iii) application of a serum to the face of the subject at least twice daily following the application the toner, and in-clinic treatment comprising at least one facial acne peel or an acne hydrating treatment. In some embodiments, a sunscreen is applied daily to the face of the subject in the AM. In some embodiments, the adaptive system for clearing mild noninflamed acne in the subject comprises skin care components used in the customized skin care regimen. In some embodiments, the skin care components comprise one or more of a facial cleanser, a toner, a serum, a hydrator or moisturizer, and a broad-spectrum sunscreen. In some embodiments, the skin care components comprise a facial cleanser, a toner, a serum, a hydrator and/or a moisturizer, and a broad-spectrum sunscreen. In some embodiments, the adaptive system for clearing acne in the subject comprises instructions for the subject to carry out the customized skin care regimen. In some embodiments, the skin care components are adjusted based on the results of i) the effectiveness of acne clearance, ii) appearance of the subject's skin, and/or iii) tolerance to skin care products selected as part of the customizable skin care regimen to adapt the system to achieve a favorable outcome for acne clearance, aesthetics, and tolerance in the subject. In some embodiments, the in-clinic assessment and treatment are used to revise the customizable skin care regimen to: i) achieve favorable acne clearance; ii) improve facial skin condition in the subject; and iii) improve tolerance to skin care products selected as part of the customizable skin care regimen. In some embodiments, the cleanser is a cream facial cleanser. In some embodiments, the cream facial cleanser is a non-foaming cleanser. In some embodiments, the cream facial cleanser is an emulsifying cleanser. In some embodiments, the cream facial cleanser is an amino acid surfactant-based cleanser. In some embodiments, the cream facial cleanser is a sodium stearoyl glutamate-based cleanser. In some embodiments, the cleanser is a gel facial cleanser. In some embodiments, the gel facial cleanser is a sulfate-free gel cleanser. In some embodiments, the gel facial cleanser is a surfactant-based gel cleanser. In some embodiments, the cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the cleanser is a medicated cleanser. In some embodiments, the medicated cleanser is a medicated gel cleanser. In some embodiments, the medicated cleanser is an OTC acne gel cleanser. In some embodiments, the medicated cleanser is a medicated salicylic cleanser. In some embodiments, the toner is a soothing toner. In some embodiments, the toner is a hydrating toner. In some embodiments, the hydrating toner is a humectant-based toner. In some embodiments, the serum is a retinoid-based serum. In some embodiments, the serum is retinal-based serum. In some embodiments, the serum is a retinaldehyde serum. In some embodiments, the serum is a retinol emulsion. In some embodiments, the retinol emulsion comprises a peptide. In some embodiments, the hydrator is a hydrating gel. In some embodiments, the hydrating gel is a humectant-based gel. In some embodiments, the hydrating gel is a glycerin-based hydrating gel. In some embodiments, the hydrating gel is a hydrating repair gel serum. In some embodiments, the hydrating repair gel serum is a hyaluronic acid-based serum. In some embodiments, the hydrating repair gel serum is a beta-glucan-based serum. In some embodiments, the water-based moisturizer is a lightweight cream moisturizer. In some embodiments, the water-based moisturizer is an antioxidant-containing moisturizer. In some embodiments, the water-based moisturizer is an ester-based moisturizer. In some embodiments, the water-based moisturizer is an antioxidant-containing moisturizer. In some embodiments, the water-based moisturizer is an emollient cream moisturizer. In some embodiments, the water-based moisturizer is a peptide-containing moisturizer. In some embodiments, the subject has mild noninflamed acne, and the subject does not have inflamed acne. In some embodiments, the skin type of the subject is (i) dry, (ii) mature, or (iii) Fitzpatrick type 4, 5, or 6; wherein the cleanser is an amino acid surfactant-based cleanser or a sulfate-free gel cleanser; wherein the toner is a humectant-based toner; and wherein the serum is a retinoid-based serum. In some embodiments, the skin type of the subject is (iv) normal or (v) oily and Fitzpatrick type 4, 5, or 6, and wherein the cleanser is a surfactant-based gel cleanser; or wherein the skin type of the subject is (vi) oily, or (vii) oily P/N, and wherein the cleanser is an surfactant-based gel cleanser or a surfactant-based exfoliating gel cleanser; wherein the toner is a soothing facial toner; and wherein the serum is a retinoid-based serum. In some embodiments of methods described herein, an optional hydrator is applied to the skin of the subject. In some embodiments of methods described herein, an optional moisturizer is applied to the skin of the subject. In some embodiments, the moisturizer is an emollient cream moisturizer. In some embodiments, the moisturizer is an antioxidant-containing moisturizer. In some embodiments of methods described herein, the subject achieves a significant clearance in acne as defined by having an Investigator's Global Assessment (IGA) of acne severity score of 0. In an aspect described herein, are systems for clearing noninflamed acne in a subject in need thereof, the systems comprising: a cleanser, a toner, a serum, an optional hydrator, and an optional moisturizer, and instructions, wherein the system is configured to perform the method steps of a customized skin care regimen comprising: i) cleansing the face of the subject at least twice daily with a facial cleanser; ii) application of a toner to the face of the subject at least twice daily following the application of ice; and iii) application of a serum to the face of the subject at least twice daily following the application the toner; and in-clinic treatment comprising at least one facial acne peel or an acne hydrating treatment. In some embodiments, the customizable skin care regimen comprises applying a topical bactericidal-based acne treatment formulation to the skin of the subject following the application of the serum, wherein the topical bactericidal-based acne treatment formulation is a benzoyl peroxide-based acne treatment topical formulation. In some embodiments, the customizable skin care regimen comprises applying a topical bactericidal-based acne treatment formulation to the skin of the subject following the application of the serum, wherein the topical bactericidal-based acne treatment formulation is a salicylic acid-based acne treatment topical formulation. In some embodiments, the customizable skin care regimen comprises applying a topical bactericidal-based acne treatment formulation to the skin of the subject following the application of the serum, wherein the topical bactericidal-based acne treatment formulation is a sulfur-based acne treatment topical formulation.

### Mild Noninflamed Initial Skin Care Routine 1

Following the initial assessment of the subject, mild noninflamed initial skin care routine 1 may be started as an at-home customizable skin care regimen. In some embodiments of the system or method for clearing acne, routine 1 is appropriate for use in a subject with a dry skin type, a mature skin type, Fitzpatrick scale skin type IV, Fitzpatrick scale skin type V, Fitzpatrick scale skin type VI, or any combination thereof. In some embodiments of the system or method for clearing acne, the mild noninflamed initial skin care routine 1 for use in a subject with dry skin comprises use of a cleanser, a toner, a serum, an optional hydrator, an optional moisturizer, or a sunscreen, or any combination thereof. In some embodiments of the system or method for clearing acne, the mild noninflamed initial skin care routine 1 for use in a subject with dry skin comprises use of a cleanser, a toner, a serum, a hydrator, a moisturizer, and a sunscreen. In some embodiments of the system or method for clearing acne, the mild noninflamed initial skin care routine 1 for use in a subject with dry skin comprises use of a cleanser, a toner, a serum, an optional hydrator, an optional moisturizer, and a sunscreen. In some embodiments, the mild noninflamed initial skin care routine 1 for use in a subject with dry skin comprises use of a cleanser, a toner, a serum, an optional hydrator, an optional moisturizer, and a sunscreen, and any combination thereof.

In some embodiments of the system or method of clearing acne, a cleanser, a toner, a serum, a hydrator, a moisturizer, and a sunscreen, are each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the toner is a hydrating toner. In some embodiments, the hydrating toner is a humectant-based toner. In some embodiments, humectant-based toner comprises sodium hyaluronate. In some embodiments, the serum is a retinoid-based serum. In some embodiments, the serum is retinal-based serum. In some embodiments, the serum is a retinaldehyde serum. In some embodiments, if the subject cannot tolerate the retinaldehyde serum an alpha hydroxy acid-based serum is tested on the subject for tolerance. In some embodiments, if the subject cannot tolerate the retinaldehyde serum, the subject is switched to using a mandelic acid-based serum or a glycolic acid-based serum. In some embodiments, the serum is 5% or 8% mandelic acid-based serum. In some embodiments, the serum is 5% or 10% glycolic acid-based serum. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is at least SPF 30. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, if the subject develops inflamed acne, the subject ceases the mild noninflamed initial skin care routine and starts an initial skin care routine for inflamed or combination acne according to the skin type and skin condition of the subject.

In some embodiments of the system or method of clearing acne, the cleanser is a sodium stearoyl glutamate-based emulsifying cleanser. In some embodiments, the toner is a humectant-based toner. In some embodiments, the serum is a retinoid-based serum. In some embodiments, the serum is retinal-based serum. In some embodiments, the serum is a retinaldehyde serum. In some embodiments, the serum is changed to a retinol emulsion as needed. In some embodiments, the retinol emulsion comprises a peptide. In some embodiments, if the subject cannot tolerate the retinaldehyde serum, the serum is changed to a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the hydrator is a hydrating gel. In some embodiments, the hydrating gel is a glycerin-based hydrating gel. In some embodiments, the moisturizer is an antioxidant and peptide-containing moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50.

In some embodiments of the system or method for clearing acne, the mild noninflamed initial skin care routine 1 for use in a subject with mature skin comprises use of a cleanser, a toner, a serum, a hydrator, a moisturizer, or a sunscreen, or any combination thereof. In some embodiments of the system or method of clearing acne, the mild noninflamed initial skin care routine 1 for use in a subject with mature skin comprises use of a cleanser, a toner, a serum, an optional hydrator, an optional moisturizer, and a sunscreen. In some embodiments of the system for clearing acne, the mild noninflamed initial skin care routine 1 for use in a subject with mature skin comprises use of a cleanser, a toner, a serum, a hydrator, a moisturizer, and a sunscreen. In some embodiments of the method for clearing acne, the mild noninflamed initial skin care routine 1 for use in a subject with mature skin comprises use of a cleanser, a toner, a serum, a hydrator, a moisturizer, and a sunscreen.

In some embodiments of the system or method for clearing acne, a cleanser, a toner, a serum, a hydrator, a moisturizer, and a sunscreen are each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a hydrating toner. In some embodiments, the hydrating toner is a humectant-based toner. In some embodiments, humectant-based toner comprises sodium hyaluronate. In some embodiments, the serum is a retinoid-based serum. In some embodiments, the serum is retinal-based serum. In some embodiments, the serum is a retinaldehyde serum. In some embodiments, the serum is switched to a retinol emulsion as needed. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the broad-spectrum sunscreen is at least SPF 30. In some embodiments, the broad-spectrum sunscreen is at least SPF 50. In some embodiments, the sunscreen is applied daily to the face of the subject in the AM.

In some embodiments of the system or method for clearing acne, the noninflamed initial skin care routine 1 for use in a subject with mature skin comprises use of a cleanser, a toner, a serum, a hydrator, a moisturizer, and a sunscreen, and any combination thereof. In some embodiments of the system or method of clearing acne, the cleanser is a sodium stearoyl glutamate-based emulsifying cleanser, a surfactant-based gel cleanser, or a sulfate-free gel cleanser. In some embodiments, the toner is a humectant-based toner. In some embodiments, the serum is a retinoid-based serum. In some embodiments, the serum is retinal-based serum. In some embodiments, the serum is a retinaldehyde serum. In some embodiments, if the subject cannot tolerate the retinaldehyde serum an alpha hydroxy acid-based serum is tested on the subject for tolerance. In some embodiments, if the subject cannot tolerate the retinaldehyde serum, the subject is switched to using a mandelic acid-based serum or a glycolic acid-based serum. In some embodiments, the serum is 5% or 8% mandelic acid-based serum. In some embodiments, the serum is 5% or 10% glycolic acid-based serum. In some embodiments, the moisturizer is an antioxidant and peptide-containing moisturizer. In some embodiments, the sunscreen comprises a daily SPF 30 organic filter-based sunscreen. In some embodiments, the sunscreen comprises a chemical-based sunscreen.

In some embodiments of the system or method for clearing acne, the mild noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, a hydrator, a moisturizer, or a sunscreen, or any combination thereof. In some embodiments of the system or method of clearing acne, the mild noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, a hydrator, a moisturizer, and a sunscreen. In some embodiments of the system or method of clearing acne, the mild noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, a hydrator, a moisturizer, and a sunscreen. In some embodiments of the system or method of clearing acne, the mild noninflamed initial skin care routine 1 for use in a subject with Fitzpatrick scale skin type IV, Fitzpatrick scale skin type V, or Fitzpatrick scale skin type VI skin comprises use of a cleanser, a toner, a serum, an optional hydrator, an optional moisturizer, and a sunscreen.

In some embodiments of the system or method of clearing acne, a cleanser, a toner, a serum, a hydrator, a moisturizer, and a sunscreen, are each selected for use in the adaptive system or method. In some embodiments, the cleanser is a cream facial cleanser. In some embodiments, the cleanser is a non-foaming cleanser. In some embodiments, the cream facial cleanser or non-foaming cleanser is an amino acid surfactant-based cleanser. In some embodiments, the cream facial cleanser or non-foaming cleanser is a sodium stearoyl glutamate-based emulsifying cleanser. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a hydrating toner. In some embodiments, the hydrating toner is a humectant-based toner. In some embodiments, humectant-based toner comprises sodium hyaluronate. In some embodiments, the serum is a retinoid-based serum. In some embodiments, the serum is retinal-based serum. In some embodiments, the serum is a retinaldehyde serum. In some embodiments, if the subject cannot tolerate the retinaldehyde serum an alpha hydroxy acid-based serum is tested on the subject for tolerance. In some embodiments, if the subject cannot tolerate the retinaldehyde serum, the subject is switched to using a mandelic acid-based serum or a glycolic acid-based serum. In some embodiments, the serum is 5% or 8% mandelic acid-based serum. In some embodiments, the serum is 5% or 10% glycolic acid-based serum. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is an emollient cream moisturizer. In some embodiments, the moisturizer is an antioxidant-containing moisturizer. In some embodiments, the moisturizer is an antioxidant and peptide-containing moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the broad-spectrum sunscreen is at least SPF 30. In some embodiments, the broad-spectrum sunscreen is at least SPF 50.

In some embodiments of the system or method of clearing acne, the cleanser is a sodium stearoyl glutamate-based emulsifying cleanser, a surfactant-based gel cleanser, or a sulfate-free gel cleanser. In some embodiments, the toner is a humectant-based toner. In some embodiments, the serum is a retinaldehyde serum. In some embodiments, the hydrator is a hydrating gel. In some embodiments, the moisturizer is an antioxidant and peptide-containing moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject.

In some embodiments, for a subject with a dry skin type, a mature skin type, Fitzpatrick scale skin type IV, Fitzpatrick scale skin type V, Fitzpatrick scale skin type VI, or any combination thereof, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, optional hydrator, optional moisturizer, and sunscreen. In some embodiments, the Week 1 and Week 2 PM home care protocol comprises the following order of the application of: i) the cleanser, ii) toner, iii) hydrator, and iv) moisturizer. In some embodiments, for a subject with a dry skin type, a mature skin type, Fitzpatrick scale skin type IV, Fitzpatrick scale skin type V, Fitzpatrick scale skin type VI, or any combination thereof, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of: i) the cleanser, ii) the toner, and iii) optional hydrator. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of: i) the cleanser, ii) the toner, iii) the serum, and iv) optional hydrator. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of: i) the cleanser, ii) the toner, iii) the serum, and iv) optional hydrator. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, during Week 7 or Week 8, if total noninflamed acne count has increased since the initial assessment, the retinaldehyde serum is replaced by a retinol emulsion. In some embodiments, the retinol emulsion comprises a peptide. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) if the subject has been using the retinaldehyde serum once per day, increase application of the retinal-based serum to twice per day (once in morning and once in evening), ii) if the subject has been using the retinaldehyde serum twice per day, change the serum to twice per day (once in AM/once in PM) retinol emulsion comprising a peptide, iii) if the subject has been using the retinaldehyde serum twice per day, the subject is directed to layer a 10% glycolic acid-based serum underneath the retinaldehyde serum if appropriate for the skin type of the subject, iv) if the subject has been using the retinol emulsion comprising a peptide twice per day, the subject is directed to layer a 10% glycolic acid-based serum underneath the retinol emulsion comprising a peptide if appropriate for the skin type of the subject, v) change to a stronger cleanser or scrub, and/or vi) change to a stronger toner. If after Week 7, Week 8, or Week 9 the subject has developed inflamed acne, the subject is directed to stop the mild noninflamed initial skin care routine and start a noninflamed initial skin care routine appropriate for their skin type and skin condition. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Mild Noninflamed Initial Skin Care Routine 2

Following the initial assessment of the subject, mild noninflamed initial skin care routine 2 may be started as an at-home customizable skin care regimen. In some embodiments of the system or method for clearing acne, routine 2 is appropriate for use in a subject with a normal skin type. In some embodiments of the system or method for clearing acne, the mild noninflamed initial skin care routine 2 for use in a subject with normal skin comprises use of a cleanser, a toner, a serum, a hydrator, a moisturizer, or a sunscreen, or any combination thereof. In some embodiments of the system or method for clearing acne, the mild noninflamed initial skin care routine 2 for use in a subject with normal skin comprises use of a cleanser, a toner, a serum, a hydrator, a moisturizer, and a sunscreen. In some embodiments of the system for clearing acne, the mild noninflamed initial skin care routine 2 for use in a subject with normal skin comprises use of a cleanser, a toner, a serum, an optional hydrator, an optional moisturizer, and a sunscreen. In some embodiments, if the subject develops inflamed acne, the subject ceases the noninflamed initial skin care routine and starts an initial skin care routine for inflamed or combination acne according to the skin type and skin condition of the subject.

In some embodiments of the system or method for clearing acne, a cleanser, a toner, a serum, a hydrator, a moisturizer, and a sunscreen are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a soothing toner. In some embodiments, the serum is a retinoid-based serum. In some embodiments, the serum is retinal-based serum. In some embodiments, the serum is a retinaldehyde serum. In some embodiments, if the subject cannot tolerate the retinaldehyde serum an alpha hydroxy acid-based serum is tested on the subject for tolerance. In some embodiments, if the subject cannot tolerate the retinaldehyde serum, the subject is switched to using a mandelic acid-based serum or a glycolic acid-based serum. In some embodiments, the serum is 5% or 8% mandelic acid-based serum. In some embodiments, the serum is 5% or 10% glycolic acid-based serum. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the water-based moisturizer is a lightweight cream moisturizer. In some embodiments, the water-based moisturizer is an antioxidant-containing moisturizer. In some embodiments, the water-based moisturizer is an ester-based moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the broad-spectrum sunscreen is at least SPF 30. In some embodiments, the broad-spectrum sunscreen is at least SPF 50. In some embodiments, the sunscreen is hybrid organic and inorganic-based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is applied daily to the face of the subject in the AM.

In some embodiments of the system or method for clearing acne, the cleanser is a sulfate-free gel cleanser. In some embodiments, the toner is a soothing facial toner. In some embodiments, the serum is a retinoid-based serum. In some embodiments, the serum is retinal-based serum. In some embodiments, the serum is a retinaldehyde serum. In some embodiments, the serum is changed to a retinol emulsion as needed. In some embodiments, the retinol emulsion comprises a peptide. In some embodiments, if the subject cannot tolerate the retinaldehyde scrum, the serum is changed to a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the hydrator is a hydrating gel. In some embodiments, the moisturizer is an ester-based moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50.

In some embodiments of the system or method for clearing acne, in home care protocols are assigned on a Week basis and may vary or be adjusted after each week, after Week 2, after Week 4, after Week 6, after Week 7, or after any subsequent week after Week 7 in which the in-home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, for a subject with a normal skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of: i) the cleanser, ii) the toner, iii) hydrator, and iv) the moisturizer. In some embodiments, for a subject with a normal skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of: i) the cleanser, ii) the toner, iii) serum, and iv) optional hydrator, and v) optional moisturizer. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of: i) the cleanser, ii) the toner, iii) the serum, iv) optional hydrator, and v) optional moisturizer. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of: i) the cleanser, ii) the toner, iii) the serum, iv) optional hydrator, and v) optional moisturizer. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinaldehyde serum is replaced by a retinol emulsion. In some embodiments, the retinol emulsion comprises a peptide. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) if the subject has been using the retinaldehyde serum once per day, increase application of the retinal-based serum to twice per day (once in morning and once in evening), ii) if the subject has been using the retinaldehyde serum twice per day, change the serum to twice per day (once in AM/once in PM) retinol emulsion comprising a peptide, iii) if the subject has been using the retinaldehyde serum twice per day, the subject is directed to layer a 10% glycolic acid-based serum underneath the retinaldehyde serum if appropriate for the skin type of the subject, iv) if the subject has been using the retinol emulsion comprising a peptide twice per day, the subject is directed to layer a 10% glycolic acid-based serum underneath the retinol emulsion comprising a peptide if appropriate for the skin type of the subject, v) change to a stronger cleanser or scrub, and/or vi) change to a stronger toner. If after Week 7, Week 8, or Week 9 the subject has developed inflamed acne, the subject is directed to stop the mild noninflamed initial skin care routine and start a noninflamed initial skin care routine appropriate for their skin type and skin condition. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Mild Noninflamed Initial Skin Care Routine 3

Following the initial assessment of the subject, mild noninflamed initial skin care routine 3 may be started as an at-home customizable skin care regimen. In some embodiments of the system or method for clearing acne, routine 3 is appropriate for use in a subject with oily skin also determined to have Fitzpatrick scale skin type IV, Fitzpatrick scale skin type V, or Fitzpatrick scale skin type VI skin type. In some embodiments of the system or method for clearing acne, the mild noninflamed initial skin care routine 3 is for use in a subject with oily skin also determined to have Fitzpatrick scale skin type IV, Fitzpatrick scale skin type V, or Fitzpatrick scale skin type VI skin type comprises use of a cleanser, a toner, a serum, a hydrator, a moisturizer, or a sunscreen, or any combination thereof. In some embodiments of the system or method for clearing acne, the mild noninflamed initial skin care routine 3 for use in a subject with oily skin also determined to have Fitzpatrick scale skin type IV, Fitzpatrick scale skin type V, or Fitzpatrick scale skin type VI skin type comprises use of a cleanser, a toner, a serum, a hydrator, a moisturizer, and a sunscreen. In some embodiments of the system or method for clearing acne, the mild noninflamed initial skin care routine 3 for use in a subject with oily skin also determined to have Fitzpatrick scale skin type IV, Fitzpatrick scale skin type V, or Fitzpatrick scale skin type VI skin type comprises use of a cleanser, a toner, a serum, an optional hydrator, an optional moisturizer, and a sunscreen.

In some embodiments of the system or method for clearing acne, a cleanser, a toner, a serum, a hydrator, a moisturizer, and a sunscreen, are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the toner is a soothing toner. In some embodiments, the serum is a retinoid-based serum. In some embodiments, the serum is retinal-based serum. In some embodiments, the serum is a retinaldehyde serum. In some embodiments, if the subject cannot tolerate the retinaldehyde serum an alpha hydroxy acid-based serum is tested on the subject for tolerance. In some embodiments, if the subject cannot tolerate the retinaldehyde serum, the subject is switched to using a mandelic acid-based serum or a glycolic acid-based serum. In some embodiments, the serum is 5% or 8% mandelic acid-based serum. In some embodiments, the serum is 5% or 10% glycolic acid-based serum. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an organic filter-based sunscreen. In some embodiments, the broad-spectrum sunscreen is a chemical-based sunscreen. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the broad-spectrum sunscreen is at least SPF 30. In some embodiments, the broad-spectrum sunscreen is at least SPF 50. In some embodiments, the sunscreen is a combination mineral and chemical-based sunscreen. In some embodiments, the sunscreen is hybrid organic and inorganic-based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, if the subject develops inflamed acne, the subject ceases the mild noninflamed initial skin care routine and starts an initial skin care routine for inflamed or combination acne according to the skin type and skin condition of the subject.

In some embodiments of the system or method for clearing acne, the cleanser is a sulfate-free gel cleanser. In some embodiments, the toner is a soothing facial toner. In some embodiments, the serum is a retinoid-based serum. In some embodiments, the serum is retinal-based serum. In some embodiments, the serum is a retinaldehyde serum. In some embodiments, the serum is changed to a retinol emulsion as needed. In some embodiments, the retinol emulsion comprises a peptide. In some embodiments, if the subject cannot tolerate the retinaldehyde serum, the serum is changed to a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the hydrator is a hydrating gel. In some embodiments, the moisturizer is an ester-based moisturizer. In some embodiments, the sunscreen comprises a daily SPF 30 organic filter-based sunscreen. In some embodiments, the sunscreen comprises a chemical-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, for a subject with oily skin also determined to have Fitzpatrick scale skin type IV, Fitzpatrick scale skin type V, or Fitzpatrick scale skin type VI skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of: i) the cleanser, ii) the toner, iii) the hydrator, and iv) the moisturizer. In some embodiments, for a subject with oily skin also determined to have Fitzpatrick scale skin type IV, Fitzpatrick scale skin type V, or Fitzpatrick scale skin type VI skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of: i) the cleanser, ii) the toner, iii) the serum, and iv) optional hydrator, and v) optional moisturizer. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of: i) the cleanser, ii) the toner, iii) the serum, iv) optional hydrator, and v) optional moisturizer. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of: i) the cleanser, ii) the toner, iii) the serum, iv) optional hydrator, and v) optional moisturizer. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based scrum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinaldehyde serum is replaced by a retinol emulsion. In some embodiments, the retinol emulsion comprises a peptide. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) if the subject has been using the retinaldehyde serum once per day, increase application of the retinal-based serum to twice per day (once in morning and once in evening), ii) if the subject has been using the retinaldehyde serum twice per day, change the serum to twice per day (once in AM/once in PM) retinol emulsion comprising a peptide, iii) if the subject has been using the retinaldehyde serum twice per day, the subject is directed to layer a 10% glycolic acid-based serum underneath the retinaldehyde serum if appropriate for the skin type of the subject, iv) if the subject has been using the retinol emulsion comprising a peptide twice per day, the subject is directed to layer a 10% glycolic acid-based serum underneath the retinol emulsion comprising a peptide if appropriate for the skin type of the subject, v) change to a stronger cleanser or scrub, and/or vi) change to a stronger toner. If after Week 7, Week 8, or Week 9 the subject has developed inflamed acne, the subject is directed to stop the mild noninflamed initial skin care routine and start a noninflamed initial skin care routine appropriate for their skin type and skin condition. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Mild Noninflamed Initial Skin Care Routine 4

Following the initial assessment of the subject, mild noninflamed initial skin care routine 4 may be started as an at-home customizable skin care regimen. In some embodiments of the system or method for clearing acne, routine 4 is appropriate for use in a subject with oily skin irrespective of Fitzpatrick scale skin type. In some embodiments of the system or method for clearing acne, the mild noninflamed initial skin care routine 4 for use in a subject with oily skin irrespective of Fitzpatrick scale skin type comprises use of a cleanser, a toner, a serum, a hydrator, a moisturizer, or a sunscreen, or any combination thereof. In some embodiments of the system or method for clearing acne, the mild noninflamed initial skin care routine 4 for use in a subject with oily skin irrespective of Fitzpatrick scale skin type comprises use of a cleanser, a toner, a serum, a hydrator, a moisturizer, and a sunscreen. In some embodiments of the system for clearing acne, the mild noninflamed initial skin care routine 4 for use in a subject with oily skin irrespective of Fitzpatrick scale skin type comprises use of a cleanser, a toner, a serum, an optional hydrator, an optional moisturizer, and a sunscreen.

In some embodiments of the system or method for clearing acne, a cleanser, a toner, a serum, a hydrator, a moisturizer, and a sunscreen are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the facial cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the toner is a soothing toner. In some embodiments, the serum is a retinoid-based serum. In some embodiments, the serum is retinal-based serum. In some embodiments, the serum is a retinaldehyde serum. In some embodiments, if the subject cannot tolerate the retinaldehyde serum an alpha hydroxy acid-based serum is tested on the subject for tolerance. In some embodiments, if the subject cannot tolerate the retinaldehyde serum, the subject is switched to using a mandelic acid-based serum or a glycolic acid-based serum. In some embodiments, the serum is 5% or 8% mandelic acid-based serum. In some embodiments, the serum is 5% or 10% glycolic acid-based serum. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an inorganic-filter based sunscreen. In some embodiments, the broad-spectrum sunscreen is a mineral-based sunscreen. In some embodiments, the mineral-based sunscreen is a zinc oxide-based sunscreen. In some embodiments, the broad-spectrum sunscreen is at least SPF 28. In some embodiments, the broad-spectrum sunscreen is a mineral-based sunscreen. In some embodiments, the broad-spectrum sunscreen is at least SPF 50. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, if the subject develops inflamed acne, the subject ceases the mild noninflamed initial skin care routine and starts an initial skin care routine for inflamed or combination acne according to the skin type and skin condition of the subject.

In some embodiments of the system or method for clearing acne, the cleanser is a sulfate-free gel cleanser or a mandelic acid-based cleanser. In some embodiments, the toner is a soothing toner. In some embodiments, the serum is a retinoid-based serum. In some embodiments, the serum is retinal-based serum. In some embodiments, the serum is a retinaldehyde serum. In some embodiments, the serum is changed to a retinol emulsion as needed. In some embodiments, the retinol emulsion comprises a peptide. In some embodiments, if the subject cannot tolerate the retinaldehyde serum, the serum is changed to a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the hydrator is a hydrating gel. In some embodiments, the moisturizer is an ester-based moisturizer. In some embodiments, the sunscreen comprises a daily SPF 28 zinc-oxide based sunscreen. In some embodiments, the sunscreen comprises an SPF 50 mineral-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, for a subject with oily skin irrespective of Fitzpatrick scale skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of: i) the cleanser, ii) the toner, iii) the hydrator, and iv) the moisturizer. In some embodiments, for a subject with oily skin irrespective of Fitzpatrick scale skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of: i) the cleanser, ii) the toner, iii) the serum, and iv) optional hydrator, and v) optional moisturizer. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of: i) the cleanser, ii) the toner, iii) the serum, iv) optional hydrator, and v) optional moisturizer. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of: i) the cleanser, ii) the toner, iii) the serum, iv) optional hydrator, and v) optional moisturizer. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinaldehyde serum is replaced by a retinol emulsion. In some embodiments, the retinol emulsion comprises a peptide. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) if the subject has been using the retinaldehyde serum once per day, increase application of the retinal-based serum to twice per day (once in morning and once in evening), ii) if the subject has been using the retinaldehyde serum twice per day, change the serum to twice per day (once in AM/once in PM) retinol emulsion comprising a peptide, iii) if the subject has been using the retinaldehyde serum twice per day, the subject is directed to layer a 10% glycolic acid-based serum underneath the retinaldehyde serum if appropriate for the skin type of the subject, iv) if the subject has been using the retinol emulsion comprising a peptide twice per day, the subject is directed to layer a 10% glycolic acid-based serum underneath the retinol emulsion comprising a peptide if appropriate for the skin type of the subject, v) change to a stronger cleanser or scrub, and/or vi) change to a stronger toner. In some embodiments, the sun protection is a broad-spectrum sunscreen. In some embodiments, the broad-spectrum sunscreen is an inorganic-filter sunscreen. In some embodiments, the broad-spectrum sunscreen is a mineral-based sunscreen. In some embodiments, mineral-based sunscreen is a zinc oxide-based sunscreen. In some embodiments, the broad-spectrum sunscreen is rated at about Sun Protection Factor (SPF) 28. In some embodiments, the broad-spectrum sunscreen is rated at about SPF 40. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the sunscreen is applied daily to the face of the subject in the AM. In some embodiments, the optional hydrator is applied to the skin of the subject and maintains skin of the subject in a healthy state of hydration so that excess oil is not produced.

### Mild Noninflamed Initial Skin Care Routine 5

Following the initial assessment of the subject, mild noninflamed initial skin care routine 5 may be started as an at-home customizable skin care regimen. In some embodiments of the system or method for clearing acne, routine 5 is appropriate for use in a subject with oily skin that is pregnant or nursing (oily P/N). In some embodiments of the system or method for clearing acne, the mild noninflamed initial skin care routine 5 for use in a subject with oily skin that is pregnant or nursing comprises use of a cleanser, a toner, a serum, a hydrator, a moisturizer, or a sunscreen, or any combination thereof. In some embodiments of the system or method for clearing acne, the mild noninflamed initial skin care routine 5 for use in a subject with oily skin that is pregnant or nursing comprises use of a cleanser, a toner, a serum, a hydrator, a moisturizer, and a sunscreen. In some embodiments of the system or method for clearing acne, the mild noninflamed initial skin care routine 5 for use in a subject with oily skin that is pregnant or nursing comprises use of a cleanser, a toner, a serum, an optional hydrator, an optional moisturizer, and a sunscreen.

In some embodiments of the system or method for clearing acne, a cleanser, a toner, a serum, a hydrator, a moisturizer, and a sunscreen are each selected for use in the adaptive system or method. In some embodiments, the cleanser is a gel facial cleanser. In some embodiments, the gel facial cleanser is a surfactant-based gel cleanser. In some embodiments, the gel facial cleanser is a sulfate-free gel cleanser. In some embodiments, the cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the toner is a soothing toner. In some embodiments, the serum is a retinoid-based serum. In some embodiments, the serum is retinal-based serum. In some embodiments, the serum is a retinaldehyde serum. In some embodiments, if the subject cannot tolerate the retinaldehyde serum an alpha hydroxy acid-based serum is tested on the subject for tolerance. In some embodiments, if the subject cannot tolerate the retinaldehyde serum, the subject is switched to using a mandelic acid-based serum or a glycolic acid-based serum. In some embodiments, the serum is 5% or 8% mandelic acid-based serum. In some embodiments, the serum is 5% or 10% glycolic acid-based serum. In some embodiments, the hydrator is a hydrating gel. In some embodiments, the hydrating gel is a hydrating repair gel serum. In some embodiments, the hydrating gel is a humectant-based gel. In some embodiments, the hydrating gel is a hyaluronic based serum. In some embodiments, the hydrating gel is a beta-glucan based serum. In some embodiments, the moisturizer is a water-based moisturizer. In some embodiments, the moisturizer is a lightweight cream moisturizer. In some embodiments, the moisturizer is an ester-based moisturizer. In some embodiments, the sunscreen is a broad-spectrum sunscreen. In some embodiments, the broad-spectrum sunscreen is an inorganic-filter based sunscreen. In some embodiments, the broad-spectrum sunscreen is a mineral-based sunscreen. In some embodiments, the mineral-based sunscreen is a zinc oxide-based sunscreen. In some embodiments, the broad-spectrum sunscreen is a non-comedogenic sunscreen. In some embodiments, the non-comedogenic sunscreen is at least SPF 30. In some embodiments, the non-comedogenic sunscreen is at least SPF 50. In some embodiments, the broad-spectrum sunscreen is at least SPF 28. In some embodiments, the broad-spectrum sunscreen is a mineral-based sunscreen. In some embodiments, the broad-spectrum sunscreen is at least SPF 40. In some embodiments, the sunscreen is a combination mineral and chemical-based sunscreen. In some embodiments, the sunscreen is hybrid organic and inorganic based sunscreen. In some embodiments, the combination mineral and chemical-based sunscreen is at least SPF 15. In some embodiments, the combination mineral and chemical-based sunscreen is at least SPF 30. In some embodiments, the combination mineral and chemical-based sunscreen is at least SPF 50. In some embodiments, if the subject develops inflamed acne, the subject ceases the mild noninflamed initial skin care routine and starts an initial skin care routine for inflamed or combination acne according to the skin type and skin condition of the subject.

In some embodiments of the system or method for clearing acne, the cleanser is a sulfate-free gel cleanser or a mandelic acid-based cleanser. In some embodiments, the toner is a soothing toner. In some embodiments, the serum is a retinoid-based serum. In some embodiments, the serum is retinal-based serum. In some embodiments, the serum is a retinaldehyde serum. In some embodiments, the serum is changed to a retinol emulsion as needed. In some embodiments, the retinol emulsion comprises a peptide. In some embodiments, if the subject cannot tolerate the retinaldehyde serum, the serum is changed to a 5% mandelic acid-based serum or an 8% mandelic acid-based serum. In some embodiments, if the subject cannot tolerate mandelic acid-based serum, an alpha-hydroxy-based serum is tried on the subject for skin tolerance. In some embodiments, the alpha-hydroxy-based serum is a glycolic acid-based serum. In some embodiments, the glycolic acid-based serum is a 5% glycolic serum. In some embodiments, the glycolic acid-based serum is a 10% glycolic serum. In some embodiments, the hydrator is a hydrating gel. In some embodiments, the moisturizer is an ester-based moisturizer. In some embodiments, the sunscreen comprises a daily SPF 28 zinc-oxide based sunscreen. In some embodiments, the sunscreen comprises an SPF 50 mineral-based sunscreen. In some embodiments, the sunscreen is a non-comedogenic sunscreen. In some embodiments, the non-comedogenic sunscreen is at least SPF 30. In some embodiments, the non-comedogenic sunscreen is at least SPF 50.

In some embodiments of the system or method for clearing acne, in home care protocols are assigned on a Week basis and may vary or be adjusted after each week, after Week 1, after Week 2, after Week 3, after Week 4, after Week 5, after Week 6, after Week 7, or after any subsequent week after Week 7 in which the in-home protocols are still being conducted by the subject. In some embodiments, for a subject with oily skin irrespective of Fitzpatrick scale skin type, Week 1 and Week 2 of the customizable skin care regimen comprises a daily morning (AM) and a daily evening (PM) home care protocol. In some embodiments, the Week 1 and Week 2 AM home care protocol comprises application of the cleanser, toner, serum every other day, moisturizer, and sunscreen. In some embodiments, the Week 1 and Week 2 AM home care protocol comprises optional application of hydrator. In some embodiments, the Week 1 and Week 2 PM home care protocol comprises the following order of the application of: i) the cleanser, ii) the toner, iii) the hydrator, and iv) the moisturizer. In some embodiments, for a subject with oily skin irrespective of Fitzpatrick scale skin type, Week 3 and Week 4 of the customizable skin care regimen comprises a daily morning (AM) and an evening (PM) home care protocol. In some embodiments, the Week 3 and Week 4 AM home care protocol comprises application of the cleanser, toner, serum, optional hydrator, optional moisturizer, and sunscreen. In some embodiments, the Week 3 and Week 4 PM home care protocol comprises the following order of the application of: i) the cleanser, ii) the toner, iii) the serum, and iv) optional hydrator, and v) optional moisturizer. In some embodiments, the Week 5 and Week 6 AM home care protocol comprises application of cleaner, toner, serum, optional hydrator, optional moisturizer, and sunscreen. In some embodiments, the Week 5 and Week 6 PM home care protocol comprises the following order of the application of: i) the cleanser, ii) the toner, iii) the serum, iv) optional hydrator, and v) optional moisturizer. In some embodiments, the Week 7 and subsequent Weeks of AM home care protocol comprises application of cleanser, toner, serum, optional hydrator, optional moisturizer, and sunscreen. In some embodiments, the Week 7 and subsequent Weeks of PM home care protocol comprises the following order of the application of: i) the cleanser, ii) the toner, iii) the serum, iv) optional hydrator, and v) optional moisturizer. In some embodiments, a hydrating mask is applied to the face of the subject as needed as part of the at-home customizable skin care regimen when the facial skin of the subject is excessively dry. In some embodiments, the hydrating mask comprises sodium hyaluronate. In some embodiments, on treatment days of in-clinic skin treatment during which the subject does not receive an acid-based chemical peel, the subject is treated in clinic with an enzyme-based chemical peel. In some embodiments, the enzyme-based chemical peel is an enzyme-containing hydrating mask. In some embodiments, the enzyme-based chemical peel is a papain-containing hydrating mask. In some embodiments, adjustments are made in Week 1, Week 2, Week 3, Week 4, Week 5, Week 6, Week 7, Week 8, or any subsequent Week to the customizable skin care regimen. In some embodiments of the system or method for clearing acne, in home care protocols are assigned on Week 1 and may vary or be adjusted at or about Week 3, at or about Week 5, at or about Week 7, or after any subsequent week after Week 7 in which the home care protocols are still being conducted by the subject. In some embodiments, the adjustments are made following an assessment by a skin care clinician. In some embodiments, the retinoid-based serum corrective serum is a retinal-based serum. In some embodiments, the retinoid-based serum corrective serum is a retinaldehyde serum. In some embodiments, the retinoid-based serum corrective serum is a retinol emulsion. In some embodiments, during Week 7 or Week 8, if total noninflamed acne count has increased since the initial assessment, the retinaldehyde serum is replaced by a retinol emulsion. In some embodiments, the retinol emulsion comprises a peptide. In some embodiments, during Week 7, if total noninflamed acne count is less than at the initial assessment, no changes made to the customizable skin care regimen. In some embodiments, during Week 8, if total noninflamed acne count is less than at the initial assessment, no changes made to the customizable skin care regimen. In some embodiments, when assessed at Week 9 or at any subsequent Week, if the subject still has a noninflamed acne count at or higher than at the initial assessment, any of the following updates to the customizable skin care regimen are considered for implementing that would be appropriate for the skin type of the subject: i) if the subject has been using the retinaldehyde serum once per day, increase application of the retinal-based serum to twice per day (once in morning and once in evening), ii) if the subject has been using the retinaldehyde serum twice per day, change the serum to twice per day (once in AM/once in PM) retinol emulsion comprising a peptide, iii) if the subject has been using the retinaldehyde serum twice per day, the subject is directed to layer a 10% glycolic acid-based serum underneath the retinaldehyde serum if appropriate for the skin type of the subject, iv) if the subject has been using the retinol emulsion comprising a peptide twice per day, the subject is directed to layer a 10% glycolic acid-based serum underneath the retinol emulsion comprising a peptide if appropriate for the skin type of the subject, v) change to a stronger cleanser or scrub, and/or vi) change to a stronger toner. In some embodiments, the sun protection is a broad-spectrum sunscreen. In some embodiments, the broad-spectrum sunscreen is an inorganic-filter sunscreen. In some embodiments, the broad-spectrum sunscreen is a mineral-based sunscreen. In some embodiments, mineral-based sunscreen is a zinc oxide-based sunscreen. In some embodiments, the broad-spectrum sunscreen is rated at about SPF 28. In some embodiments, the broad-spectrum sunscreen is rated at about SPF 40. In some embodiments, the sunscreen is applied daily to the face of the subject in the AM. In some embodiments, the optional hydrator is applied to the skin of the subject and maintains skin of the subject in a healthy state of hydration so that excess oil is not produced.

Systems and methods described above for home care recommendations for subjects having mild noninflamed acne and undergoing an initial skin care routine 1-5, may be combined with in-clinic skin treatment. In some embodiments, in-clinic skin treatment comprises application of a facial acne peel. In some embodiments, in-clinic skin treatment is applied during the initial assessment. In some embodiments, in-clinic skin treatment is applied after the initial assessment. In some embodiments, in-clinic skin treatment is applied at any one of Week 1, Week 3, Week 5, Week 7, Week 9, Week 11, Week 13, Week 15, or any Week thereafter. In some embodiments, in-clinic skin treatment is applied at Week 1, Week 3, Week 5, Week 7, Week 9, Week 11, Week 13, and Week 15. In some embodiments, in-clinic skin treatment is applied at Week 1, Week 3, Week 5, Week 7, Week 9, Week 11, Week 13, Week 15, and at any subsequent weeks in which a facial acne peel is deemed appropriate by the clinician during a follow-up assessment. In some embodiments, at Week 1 in-clinic treatment, if the clinician determines severity of 0, 1, or 2 on a scale of 0-10 of tingling, stinging, and/or burning in a skin tolerance test with use of the exfoliating toner, acid-based chemical peel 1 is performed at Week 1. In some embodiments, the acid-based chemical peel 1 is a superficial acid-based chemical peel 1. In some embodiments, the superficial acid-based chemical peel 1 is a trichloroacetic acid (TCA)-based peel. In some embodiments, the superficial acid-based chemical peel 1 is a TCA and lactic acid-based peel. In some embodiments, the superficial acid-based chemical peel 1 is a TCA and mandelic acid-based peel. In some embodiments, the superficial acid-based chemical peel 1 comprises between about 15% to about 40% acid, wherein the about 15% to about 40% acid comprises TCA, alpha hydroxy acids, salicylic acid, or kojic acid, or any combination thereof. In some embodiments, the superficial acid-based chemical peel comprises ones or more enzymes. In some embodiments, at Week 1 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 3 in-clinic treatment, if the clinician determines severity of 0, 1, or 2 on a scale of 0-10 of tingling, stinging, and/or burning in a skin tolerance test with use of the exfoliating toner, acid-based chemical peel 1 is performed at Week 3. In some embodiments, at Week 3 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 5 in-clinic treatment, if the clinician determines severity of 0, 1, or 2 on a scale of 0-10 of tingling, stinging, and/or burning in a skin tolerance test with use of the exfoliating toner, acid-based chemical peel 1 is performed at Week 5. In some embodiments, at Week 5 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 7 in-clinic treatment, if the clinician determines severity of 0, 1, or 2 on a scale of 0-10 of tingling, stinging, and/or burning in a skin tolerance test with use of the exfoliating toner, acid-based chemical peel 2 is performed at Week 7. In some embodiments, the acid-based chemical peel 2 is a TCA-based peel. In some embodiments, the acid-based chemical peel 2 is a TCA and lactic acid-based peel. In some embodiments, the acid-based chemical peel 2 is a TCA and mandelic acid-based peel. In some embodiments, at Week 7 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 9 in-clinic treatment, if the clinician determines severity of 0, 1, or 2 on a scale of 0-10 of tingling, stinging, and/or burning in a skin tolerance test with use of the exfoliating toner, acid-based chemical peel 2 is performed at Week 9. In some embodiments, at Week 9 in-clinic treatment, if the clinician determines severity of 3 or greater on a scale of 0-10 of tingling, stinging, and/or burning in a skin tolerance test with use of the exfoliating toner, acne hydrating treatment is performed. In some embodiments, at Week 11 in-clinic treatment, if the clinician determines severity of 0, 1, or 2 on a scale of 0-10 of tingling, stinging, and/or burning in a skin tolerance test with use of the exfoliating toner, acid-based chemical peel 2 or acid-based chemical peel 3 is performed at Week 11. In some embodiments, the acid-based chemical peel 3 is a TCA-based peel. In some embodiments, the acid-based chemical peel 3 is a TCA and lactic acid-based peel. In some embodiments, the acid-based chemical peel 3 is a TCA and mandelic acid-based peel. In some embodiments, at Week 11 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 13 in-clinic treatment, if the clinician determines severity of 0, 1, or 2 on a scale of 0-10 of tingling, stinging, and/or burning in a skin tolerance test with use of the exfoliating toner, acid-based chemical peel 2 or acid-based chemical peel 3 is performed at Week 13. In some embodiments, at Week 13 in-clinic treatment, if the clinician determines severity of 3 or greater on a scale of 0-10 of tingling, stinging, and/or burning in a skin tolerance test with use of the glycolic-lactic toner, acne hydrating treatment is performed. In some embodiments, at Week 15 or any subsequent week in-clinic treatment, if the clinician determines severity of 0, 1, or 2 on a scale of 0-10 of tingling, stinging, and/or burning in a skin tolerance test with use of the exfoliating toner, acid-based chemical peel 2 or acid-based chemical peel 3 is performed at Week 15 or any subsequent week in-clinic treatment. In some embodiments, at Week 15 or any subsequent week in-clinic treatment, if the clinician determines severity of 3 or greater on a scale of 0-10 of tingling, stinging, and/or burning in a skin tolerance test with use of the exfoliating toner, acne hydrating treatment is performed.

### Facial Care Products

In an aspect described herein are systems and methods of a customizable skin care regimen for use at-home for acne clearance in a subject having either: 1) mild, moderate, or severe inflamed or combination facial acne, or 2) moderate or severe noninflamed facial acne. These systems and methods employ the use of various facial care products to be used in the customizable skin care regimen. In some embodiments of systems and methods of acne clearance described herein, the subject applies an advanced topical formulation of micronized benzoyl peroxide described herein as an active ingredient to clear acne. In some embodiments of systems and methods of acne clearance described herein, the subject applies a variety of facial care products in addition to the advanced topical formulation of micronized benzoyl peroxide as part of a customizable skin care regimen. In some embodiments, the variety of facial care products are applied by the subject at-home according to the customized instructions provided by the clinician during an in-clinic assessment and treatment. In some embodiments, the variety of facial care products comprise one or more cleaners, one or more toners, one or more serums, one or more hydrators, one or more moisturizers, and one or more sun protectors. In some embodiments, in addition to the variety of facial care products, the subject is instructed to one or more acne medications. In some embodiments, the one or more acne medications comprise an advanced topical formulation of micronized benzoyl peroxide. In some embodiments, the one or more acne medications comprise a sulfur spot treatment medication. In some embodiments, the one or more acne medications are over the counter products.

### Cleansers

Systems and methods of acne clearance of customizable skin care regimens described herein comprise daily application of one or more facial cleansers as directed by a specific customizable skin care regimen. In some embodiments, the one or more facial cleansers aid in removing dirt and oils from facial skin pores. In some embodiments, the facial cleanser is a facial skin cleanser, wherein the facial skin cleanser aids in removing dirt and oils from facial skin pores. In some embodiments, the one or more facial cleansers aid in removing dirt, sebum, oil, ingredients from cosmetic products, bacteria, and exfoliated skin cells. In some embodiments, the facial skin cleanser aids in removing dirt, sebum, oil, ingredients from cosmetic products, bacteria, and exfoliated skin cells. In some embodiments, the specific customizable skin care regime is first determined at the initial assessment and one or more facial cleansers are assigned to the subject for at-home use as part of the specific customizable skin care regimen. In some embodiments, the specific customizable skin care regime is first determined during the initial in-clinic treatment and one or more facial cleansers are assigned to the subject for at-home use as part of the specific customizable skin care regimen. In some embodiments, the specific customizable skin care regime is modified depending on the response of the subject's facial skin during one or more in-clinic assessments and treatment that follow the initial in-clinic treatment and one or more facial cleansers are assigned to the subject for continued or new at-home use as part of the specific customizable skin care regimen. In some embodiments, the facial cleanser comprise one or more surface-active substances (e.g., detergents, soaps, surfactants, and/or emulsifiers) that lower the surface tension on skin upon application and remove dirt, sebum, oil, ingredients from cosmetic products, bacteria, and exfoliated skin cells. In some embodiments, the facial cleanser cleans without damaging or irritating the facial skin. In some embodiments, the facial cleanser is a gel facial cleanser. In some embodiments, the gel facial cleanser is a sulfate-free gel cleanser. In some embodiments, the gel facial cleanser is a surfactant-based gel cleanser. In some embodiments, the facial cleanser is a cream facial cleanser. In some embodiments, the cream facial cleanser is a non-foaming cleanser. In some embodiments, the cream facial cleanser is an emulsifying cleanser. In some embodiments, the cream facial cleanser is an amino acid surfactant-based cleanser. In some embodiments, the cream facial cleanser is a sodium stearoyl glutamate-based cleanser. In some embodiments, the facial cleanser is an exfoliating cleanser. In some embodiments, the exfoliating cleanser is a surfactant-based exfoliating gel cleanser. In some embodiments, the exfoliating cleanser is an alpha hydroxy gel cleanser. In some embodiments, the exfoliating cleanser is a mandelic acid-based cleanser. In some embodiments, the facial cleanser is a medicated cleanser. In some embodiments, the medicated cleanser is a medicated gel cleanser. In some embodiments, the medicated cleanser is an over the counter (OTC) acne gel cleanser. In some embodiments, the medicated cleanser is a benzoyl peroxide-based cleanser. In some embodiments, the medicated cleanser is a medicated salicylic cleanser. In some embodiments, the facial cleanser is an exfoliating cleanser. In some embodiments, the exfoliating cleanser is an antioxidant-based cleanser. In some embodiments, the facial cleanser functions as an antioxidant scrub. In some embodiments, the medicated cleanser is an exfoliating medicated cleanser. In some embodiments, the facial cleanser is a benzoyl peroxide-based cleanser.

In some embodiments, the sulfate-free cleanser comprises the following list of ingredients: Aqua (Water), Cocamidopropyl Betaine, Glycerin, Decyl Glucoside, Xanthan Gum, Phenoxyethanol, Butylene Glycol, Fomes Officinalis (Mushroom) Extract, Citric Acid, Disodium EDTA, Potassium Sorbate, Hamamelis Virginiana (Witch Hazel) Water, Camellia Sinensis (Green Tea) Leaf Extract, Glycyrrhiza Glabra (Licorice) Root Extract, Panthenol (D), Ethylhexylglycerin, and Alcohol. In some embodiments, the amino acid surfactant-based cleanser comprises the following list of ingredients: Aqua (Water), Sodium Stearoyl Glutamate, Glyceryl Stearate, Stearic Acid, Cocamidopropyl Betaine, Glycerin, Polyacrylate Crosspolymer-6, Cetyl Alcohol, Caprylic/Capric Triglyceride, Panthenol (D), Inulin, Hydrolyzed Jojoba Esters, Saccharide Isomerate, Allantoin, Bisabolol (L-alpha), Alpha-Glucan Oligosaccharide, Aloe Barbadensis Leaf Juice Powder, Centella Asiatica (Gotu Kola) Leaf Extract, Xanthan Gum, Phenoxyethanol, Ethylhexylglycerin, Sodium Hydroxide, Potassium Sorbate, Sodium Gluconate, Citric Acid, and Sodium Citrate. In some embodiments, the surfactant-based exfoliating gel cleanser comprise the following list of ingredients: Aqua (Water), Sodium C14-16 Olefin Sulfonate, Polyacrylate-1 Crosspolymer, Sodium Cocoamphoacetate, Propanediol, Hamamelis Virginiana (Witch Hazel) Water, Gluconolactone, Mandelic Acid (L) (1%), Cocamidopropyl, Hydroxysultaine, Glycerin, Sodium Benzoate, Alcohol, Honey (Mel), Camellia Oleifera (Green Tea) Leaf Extract, Vitis Vinifera (Grape) Seed Extract, and Actinidia Deliciosa (Kiwi) Fruit Extract. In some embodiments, medicated benzoyl peroxide cleanser or the OTC acne gel cleanser comprise the following list of ingredients: active ingredient: Benzoyl Peroxide at 2.5%; and inactive ingredients of: Purified Water, Ammonium Lauryl Sulfate, Glycerin, Disodium Cocoamphodiacetate, Propylene Glycol, Carbomer, and Sodium Hydroxide. In some embodiments, Antioxidant Scrub or the exfoliating cleanser comprise the following list of ingredients: Aqua (Water), Cellolose Acetate, Cocamidopropyl Betaine, Glycerin, Decyl Glucoside, Xanthan Gum, Phenoxyethanol, Butylene Glycol, Fomes Officinalis (Mushroom) Extract, Citric Acid, Disodium EDTA, Potassium Sorbate, Hamamelis Virginiana (Witch Hazel) Water, Glycyrrhiza Glabra (Licorice) Root Extract, Camellia Oleifera (Green Tea) Leaf Extract, Panthenol (D), Ethylhexylglycerin, and Alcohol. In some embodiments, the Antioxidant Scrub is packaged in 6 oz packaging for over-the-counter use. In some embodiments, the exfoliating medicated cleanser comprises the following list of ingredients: active ingredient: Benzoyl Peroxide at 2.5%; and inactive ingredients of: Purified Water, Ammonium Lauryl Sulfate, Glycerin, Cellulose Acetate, Disodium Cocoamphodiacetate, Propylene Glycol, Carbomer, and Sodium Hydroxide. In some embodiments, the benzoyl peroxidase-based cleanser comprises the following list of ingredients: ACTIVE INGREDIENT: Benzoyl Peroxide 2.5%; INACTIVE INGREDIENTS: Purified Water, Ammonium Lauryl Sulfate, Glycerin, Cellulose Acetate, Disodium Cocoamphodiacetate, Propylene Glycol, Carbomer, Sodium Hydroxide.

### Toners

Systems and methods of acne clearance of customizable skin care regimens described herein comprise daily application of one or more facial toners as directed by a specific customizable skin care regimen. In some embodiments, the specific customizable skin care regime is first determined at the initial assessment and one or more facial toners are assigned to the subject for at-home use as part of the specific customizable skin care regimen. In some embodiments, the specific customizable skin care regime is first determined during the initial in-clinic treatment and one or more facial toners are assigned to the subject for at-home use as part of the specific customizable skin care regimen. In some embodiments, the specific customizable skin care regime is modified depending on the response of the subject's facial skin during one or more in-clinic assessments and treatment that follow the initial in-clinic treatment and one or more facial toners are assigned to the subject for continued or new at-home use as part of the specific customizable skin care regimen. In some embodiments, the toner helps to balance the pH of the skin and remove impurities remaining on the skin following cleansing. In some embodiments, toner is applied to the skin of the subject after cleansing. In some embodiments, toner functions to restore pH balance to the skin after the skin's pH balance has been disrupted by application of an alkaline cleanser. In some embodiments, toner functions to remove excess oils and/or cosmetic makeup from the skin that cleansing may not successfully remove. Residual oils and/or cosmetic makeup on the skin of the subject may contribute to the formation of comedones, papules, pustules, nodules, and/or cystic acne. In some embodiments, toner functions to reduce the appearance of acne scars. In some embodiments, toner functions to enhance absorption of serums or other skincare products. In some embodiments, toner functions to prevent pores from becoming clogged. In some embodiments, toner functions to reduce inflammation which can exacerbate acne. In some embodiments, toner functions to hydrate the skin by holding water inside the surface of the skin and prevent excessive dehydration caused by an acne treatment. In some embodiments, the facial toner is a soothing toner. In some embodiments, the hydrating toner is a humectant-based toner. In some embodiments, the facial toner is as an exfoliating toner. In some embodiments, the facial toner is an exfoliating toner. In some embodiments, the exfoliating toner is a glycolic acid and lactic acid-based toner. In some embodiments, the facial toner functions as a salicylic acid-based toner. In some embodiments, the salicylic acid-containing toner is a salicylic-acid and Vitamin C-based toner. In some embodiments, the facial toner is a toning solution. In some embodiments, the facial toner is an exfoliating toner. In some embodiments, the facial toner an antioxidant-based toner. In some embodiments, the facial toner an ascorbic acid-based toner. In some embodiments, the facial toner salicylic acid-based toner or the ascorbic acid-based toner is a salicylic-acid and Vitamin C-based toner. In some embodiments, the soothing facial toner comprises the following list of ingredients: Aqua (Water), Rosa Damascena (Rose) Flower Water, Glycerin, Aloe Barbadensis Leaf Juice, Polysorbate 20, Gluconolactone, Benzyl Alcohol, Sodium Benzoate, Allantoin, Caprylhydroxamic Acid, Bisabolol, and Globularia Alypum Leaf Extract. In some embodiments, the hydrating toner comprises the following list of ingredients: Hamamelis Virginiana (Witch Hazel) Water, Aqua (Water), Alcohol, Glycerin, Polysorbate 20, Leuconostoc/Radish Root Ferment Filtrate, Sodium Lactate, Sodium PCA, Sodium Gluconate, O-cymen-5-OL, Sorbitol, Proline (L), Fructooligosaccharides (D-beta), Glucosamine HCl (D), Gluconolactone, Sodium Hyaluronate (L), Sodium Benzoate, Phenethyl Alcohol, Caprylyl Glycol, and Potassium Sorbate. In some embodiments, the glycolic acid and lactic acid-based toner comprises the following list of ingredients: Aqua (Water), Glycolic Acid, Lactic Acid (L), Propanediol, Alcohol Denat., Glycerin, Leuconostoc/Radish Root Ferment Filtrate, Hamamelis Virginiana (Witch Hazel) Water, Caprylyl Glycol, Sodium PCA, Sodium Gluconate, Chamomilla Recutita (Matricaria) Flower Extract, Alcohol, Allantoin, and Ascorbic Acid (L). In some embodiments, the salicylic-acid and Vitamin C-based toner comprises the following list of ingredients: Aqua (Water), Alcohol Denat., Glycerin, Propylene Glycol, Polysorbate 20, Gluconolactone, Salicylic Acid, Niacinamide, Benzyl Alcohol, Sodium Benzoate, Caprylhydroxamic Acid, Benzoic Acid, Salix Alba (Willow) Bark Extract, Panthenol (D), Ascorbic Acid (L), and Beta-Carotene (D).

### Serums

Systems and methods of acne clearance of customizable skin care regimens described herein comprise daily application of one or more facial serums as directed by a specific customizable skin care regimen. In some embodiments, the specific customizable skin care regime is first determined at the initial assessment and one or more facial serums are assigned to the subject for at-home use as part of the specific customizable skin care regimen. In some embodiments, the specific customizable skin care regime is first determined during the initial in-clinic treatment and one or more facial serums are assigned to the subject for at-home use as part of the specific customizable skin care regimen. In some embodiments, the specific customizable skin care regime is modified depending on the response of the subject's facial skin during one or more in-clinic assessments and treatment that follow the initial in-clinic treatment and one or more facial serums are assigned to the subject for continued or new at-home use as part of the specific customizable skin care regimen. In some embodiments, the serum is applied prior to application of the advanced topical formulation of micronized benzoyl peroxide. In some embodiments, the serum functions aid in acne clearance by unclogging pores, exfoliating, treating acne-causing bacteria, reducing inflammation, decreasing skin oil production, or minimizing the appearance of acne scars, or any combination thereof. In some embodiments, the serum is applied all over the face of the subject. As serum can induce unwanted side effects such as skin irritation and excessive dryness, application of the specific serum in the systems and methods described herein is monitored by the clinician and changes are made as needed based on the response of the subject to the customizable skin care regimen. In some embodiments, the serum functions to effectively reduce redness. In some embodiments, the serum functions to maintain skin elasticity. In some embodiments, the serum functions to lock in moisture to the skin thereby preventing excessive dryness. In some embodiments, the serum functions to repair skin by reducing an extent of acne. In some embodiments, the serum functions to aid in clearing the complexion of the skin. In some embodiments, the serum functions to improve dullness in the skin. In some embodiments, the serum functions to reduce the appearance of dark spots on the skin. In some embodiments, the serum functions to brighten the skin. Serums are absorbed rapidly following application and are to be applied directly to the surface of the skin after cleansing or following application of toner. In some embodiments, the serum is a mandelic acid-based serum. In some embodiments, the serum is an alpha hydroxy acid-based serum. In some embodiments, the alpha hydroxy acid-based serum is a serum comprising mandelic acid and lactic acid. In some embodiments, the alpha hydroxy acid-based serum or the mandelic acid-based serum comprises the following key ingredients which aid in the function of the serum: L-Mandelic acid, L-Lactic acid, sodium hyaluronate (L), and Niacinamide. In some embodiments, the alpha hydroxy acid serum comprises L-Mandelic acid in an amount ranging from 5% to 15% w/v. In some embodiments, the serum is a 5% mandelic acid-based serum. In some embodiments, the serum is an 8% mandelic acid-based serum. In some embodiments, the serum is an 11% mandelic acid-based serum. In some embodiments, the scrum is a 15% mandelic acid-based serum. In some embodiments, the 5% mandelic acid-based serum comprises the following list of ingredients: Aqua (Water), Hamamelis Virginiana (Witch Hazel) Water, Mandelic Acid (L), Alcohol, Lactic Acid (L), Butylene Glycol, PPG-3 Benzyl Ether Myristate. Alcohol Denat., Niacinamide, Polyglyceryl-4 Caprate, Sodium Lactate, Xanthan Gum, Sodium PCA, Sodium Hyaluronate (L), Urea, Phenethyl Alcohol, Caprylyl Glycol, and Potassium Sorbate. In some embodiments, the 8% mandelic acid-based serum comprises the following list of ingredients: Aqua (Water), Hamamelis Virginiana (Witch Hazel) Water, Mandelic Acid (L), Alcohol, Lactic Acid (L), Butylene Glycol, PPG-3 Benzyl Ether Myristate, Alcohol Denat., Niacinamide, Sodium Lactate, Xanthan Gum, Sodium PCA, Sodium Hyaluronate (L), Urea, Phenethyl Alcohol, Caprylyl Glycol, and Potassium Sorbate. In some embodiments, the 11% mandelic acid-based serum comprises the following list of ingredients: Aqua (Water), Hamamelis Virginiana (Witch Hazel) Water, Mandelic Acid (L), Lactic Acid (L), Alcohol, Butylene Glycol, PPG-3 Benzyl Ether Myristate, Alcohol Denat., Niacinamide, Polyglyceryl-4 Caprate, Sodium Lactate, Xanthan Gum, Sodium PCA, Sodium Hyaluronate (L), Urea, Phenethyl Alcohol, Caprylyl Glycol, and Potassium Sorbate. In some embodiments, the 15% mandelic acid-based serum comprises the following list of ingredients: Aqua (Water), Hamamelis Virginiana (Witch Hazel) Water, Mandelic Acid (L), Lactic Acid (L), Alcohol, Butylene Glycol, PPG-3 Benzyl Ether Myristate, Alcohol Denat., Niacinamide, Sodium Lactate, Xanthan Gum, Sodium PCA, Sodium Hyaluronate (L), Urea, Phenethyl Alcohol, Caprylyl Glycol, and Potassium Sorbate. In some embodiments, the serum comprises salicylic acid-based serum. In some embodiments, the serum comprises an alpha and beta-hydroxy acid-based serum. In some embodiments, the serum is a salicylic acid-based serum. In some embodiments, the alpha and beta-hydroxy acid-based serum comprises the following list of ingredients: Aqua (Water), Alcohol Denat., Butylene Glycol, Lactic Acid (L), Salicylic Acid, Dimethyl Isosorbide, Phenoxyethanol, Potassium Azeloyl Diglycinate, Hydroxyethylcellulose, Sodium Gluconate, Xanthan Gum, O-cymen-5-OL, and Ethylhexylglycerin. In some embodiments, the serum is an alpha hydroxy acid-based serum. In some embodiments, the alpha hydroxy acid-based serum is a glycolic acid-based serum. In some embodiments, the glycolic acid-based serum comprises between about 5% to about 10% w/v of glycolic acid. In some embodiments, the 5% glycolic acid-based serum comprises the following list of ingredients: Aqua (Water), Hamamelis Virginiana (Witch Hazel) Water, Alcohol Denat., Glycerin, Glycolic Acid, Alcohol, Phenoxyethanol, Hydroxyethylcellulose, Benzyl Alcohol, Plantago Lanceolata Leaf Extract, Sodium Gluconate, Caprylhydroxamic Acid, Ethylhexylglycerin, and Xanthan Gum. In some embodiments, the 10% glycolic serum comprises the following list of ingredients: Aqua (Water), Glycolic Acid, Hamamelis Virginiana (Witch Hazel) Water, Alcohol Denatured, Glycerin, Alcohol, Phenoxyethanol, Hydroxyethylcellulose, Benzyl Alcohol, Plantago Lanceolata Leaf Extract, Sodium Gluconate, Caprylhydroxamic Acid, Ethylhexylglycerin, and Xanthan Gum. In some embodiments, the serum comprises a retinoid. In some embodiments, the serum is a retinoid-based serum. In some embodiments, the retinoid-based serum is a retinaldehyde serum. In some embodiments, the retinoid-based serum is a retinal-based serum. In some embodiments, the retinal-based serum is a retinaldehyde serum. In some embodiments, the retinoid-based serum is a retinol emulsion. In some embodiments, the retinol emulsion is a retinol peptide serum. In some embodiments, the serum comprises retinal. In some embodiments, the retinal serum is termed a retinaldehyde serum. In some embodiments, use of a retinal-containing serum is more effective at clearing acne than use of a retinol-containing serum. In some embodiments, the retinal-containing serum functions to unclog pores, prevent clogging of pores, reduce acne outbreaks, improve the appearance of acne scars, or reduce inflammation, or any combination thereof. In some instances, use of a retinal-containing serum is monitored for the appearance of any of the following potential unwanted side-effects: erythema, excessive skin dryness, itching, stinging, skin discoloration, skin photosensitivity to UV light, skin swelling, or blistering. In some embodiments, the appearance of one or more unwanted side effects is assessed by the clinician and one or more changes are made to the customizable skin care regimen involving a change in the serum used by the subject. In some embodiments, the retinaldehyde serum comprises the following list of ingredients: Aqua (Water), Glycerin, Cyclodextrin, Caprylic/Capric Triglyceride, Niacinamide, Phenoxyethanol, Butylene Glycol, Glycolic Acid, Xanthan Gum, Sclerotium Gum, Propanediol, Rubus Chamaemorus (Cloud Berry) Seed Oil, Potassium Sorbate, Ethylhexylglycerin, Retinal (Retinaldehyde), Pentylene Glycol, Tetrasodium EDTA, Lonicera Japonica (Honeysuckle) Flower Extract, Mirabilis Jalapa Flower/Leaf/Stem Extract, Lonicera Caprifolium (Honeysuckle) Flower Extract, Rosmarinyl Glucoside, Caffeyl Glucoside, Gallyl Glucoside, Saccharide and Isomerate. In some embodiments, the retinol emulsion comprises the following list of ingredients: Aqua (Water), Caprylic/Capric Triglyceride, Glycerin, Dimethyl Isosorbide, Ammonium Acryloyldimethyltaurate/VP Copolymer, Tapioca Starch, Phenoxyethanol, Retinol, Polysorbate 20, Sodium Stearoyl Glutamate, Cyclodextrin, Sodium Gluconate, O-cymen-5-OL, Ethylhexylglycerin, Pentylene Glycol, Methylglucoside Phosphate, Copper Lysinate/Prolinate, Hydroxypropyl Cyclodextrin, Palmitoyl Tripeptide-38, Caprylyl Glycol, and N-Prolyl Palmitoyl Tripeptide-56 Acetate. In some embodiments, the Vitamin A Corrective II Serum is packaged in 1 oz packaging for over the counter use.

### Hydrators

In some embodiments, systems and methods of acne clearance of customizable skin care regimens described herein comprise daily application of a hydrator as directed by a specific customizable skin care regimen. In some embodiments, application of the hydrator is optional. When skin becomes dry, sebaceous glands produce more oil which may exacerbate acne. In some embodiments, use of a hydrator maintains skin of the subject in a healthy state of hydration so that excess oil is not produced. In some embodiments, the specific customizable skin care regime is first determined at the initial assessment and a hydrator is assigned to the subject for at-home use as part of the specific customizable skin care regimen. In some embodiments, the specific customizable skin care regime is first determined during the initial in-clinic treatment and a hydrator is assigned to the subject for at-home use as part of the specific customizable skin care regimen. In some embodiments, the specific customizable skin care regime is modified depending on the response of the subject's facial skin during one or more in-clinic assessments and treatment that follow the initial in-clinic treatment and a hydrator is assigned to the subject for continued or new at-home use as part of the specific customizable skin care regimen. In some instances, the clinician determines whether the assigned hydrator should be applied to the subject as part of the customizable skin care regimen. In some instances, the subject determines whether to apply the assigned hydrator as part of the customizable skin care regimen. In some embodiments, the hydrator is a water-based hydrating solution. In some embodiments, the hydrator is formulated as a gel. In some embodiments, the hydrator contains antioxidant properties and can help reduce the appearance of oily shine. In some embodiments, the hydrator helps to regulate sebum production. In some embodiments, the hydrator helps to hydrate the skin thereby regulating the sebaceous glands to produce less sebum. In some embodiments, use of the hydrator helps prevent clogged pores and acne breakouts. In some embodiments, the hydrator reduces skin inflammation. In some embodiments, the hydrator aid in repair of damaged skin. In some embodiments, the hydrator helps to maintain the barrier function of skin. In many subjects having acne, the barrier function of facial skin is compromised which exacerbates acne breakouts. In some embodiments, the hydrator functions as a humectant. In some embodiments, the hydrator comprises sodium hyaluronate. In some embodiments, the water-based hydrator attracts and binds moisture to the skin and can hydrate oily or acne-prone skin without blocking pores or causing unwanted greasiness. In some embodiments, the hydrator helps to improve skin hydration following use of skin care products that have been used to strip oils and makeup residue from the skin. In some embodiments, the hydrator is applied prior to applying a moisturizer or a sunscreen during the day. In some embodiments, the hydrator is applied prior to applying the advanced topical formulation of micronized benzoyl peroxide. In some embodiments, the hydrator is applied prior to applying the serum. In some embodiments, the hydrator is applied after serum has been applied. In some embodiments, the hydrator is applied prior to applying the serum and following the application of the serum. In some embodiments, the hydrator is selected for application based on the severity of dehydrated skin. In some embodiments, the hydrator is effective at providing extra hydration to dehydrated skin. In some embodiments, the hydrator effectively hydrates the skin and balances oils in the skin. In some embodiments, the hydrator comprises: syringa vulgaris (lilac stem cells), sodium hyaluronate, totarol, and phenyl-N-butyl nitrone (spin trap). In some embodiments, the hydrator is a hydrating gel. In some embodiments, the hydrating gel is a glycerin-based hydrating gel. In some embodiments, the hydrating gel is a humectant-based gel. In some embodiments, the hydrating gel is a hydrating repair gel serum. In some embodiments, the hydrating repair gel serum is a hyaluronic acid-based serum. In some embodiments, the hydrating repair gel serum is a beta-glucan-based serum. In some embodiments, the glycerin-based hydrating gel comprises the following list of ingredients: Aqua (Water), Glycerin, Carbomer, Maltodextrin, Phenethyl Alcohol, Caprylyl Glycol, Sodium Hydroxide, Alcohol Denat., Sodium Hyaluronate (L), Aloe Barbadensis Leaf Juice Powder, Potassium Sorbate, Syringa Vulgaris (Lilac) Leaf Cell Culture Extract, Sodium Metabisulfite, Lonicera Japonica (Honeysuckle) Flower Extract, Santalum Austrocaledonicum (Sandalwood) Wood Oil, Trisodium Ethylenediamine Disuccinate, Hamamelis Virginiana (Witch Hazel) Water, Lavandula Angustifolia (Lavender) Oil, Rosa Damascena Flower Oil, Citric Acid, Lonicera Caprifolium (Honeysuckle) Flower Extract, Cananga Odorata (Ylang Ylang) Flower Oil, Chlorophyllin-Copper Complex, Totarol, Acetyl Tributyl Citrate, Alcohol, Cinnamomum Camphora (Camphor) Bark Oil, Citrus Aurantium Dulcis (Orange) Peel Oil, and Phenyl t-Butylnitrone. In some embodiments, the beta glucan-based gel comprises the following list of ingredients: Aqua (Water), Glycerin, Beta-Glucan, Sodium Hyaluronate, Sodium Hyaluronate Crosspolymer, Sodium Acetylated Hyaluronate, Hydrolyzed Sodium Hyaluronate, Dipotassium Glycyrrhizate, Ceramide NP, Lactobacillus Ferment, Panthenol, Alpha-Glucan Oligosaccharide, Bisabolol, Sucrose Stearate, Xylitylglucoside, Anhydroxylitol, Hydrogenated Lecithin, Inulin, Xylitol, Arnica Montana Flower Extract, Madecassic Acid, Asiatic Acid, Maltodextrin, Hydrolyzed Gardenia Florida Extract, Dimethicone, Propanediol, Phenoxyethanol, Carbomer, Butylene Glycol, Caprylyl Glycol, Polyglyceryl-10 Stearate, Ethylhexylglycerin, Hexylene Glycol, Sodium Hydroxide, Sodium Phytate, and Pentylene Glycoldelete.

### Moisturizers

In some embodiments, systems and methods of acne clearance of customizable skin care regimens described herein comprise daily application of a moisturizer as directed by a specific customizable skin care regimen. In some embodiments, application of the moisturizer is optional. When skin becomes dry, sebaceous glands produce more oil which may exacerbate acne. In some embodiments, use of a moisturizer maintains skin of the subject in a healthy state of hydration so that excess oil is not produced. In some embodiments, the specific customizable skin care regime is first determined at the initial assessment and a moisturizer is assigned to the subject for at-home use as part of the specific customizable skin care regimen. In some embodiments, the specific customizable skin care regime is first determined during the initial in-clinic treatment and a moisturizer is assigned to the subject for at-home use as part of the specific customizable skin care regimen. In some embodiments, the specific customizable skin care regime is modified depending on the response of the subject's facial skin during one or more in-clinic assessments and treatment that follow the initial in-clinic treatment and a moisturizer is assigned to the subject for continued or new at-home use as part of the specific customizable skin care regimen. In some instances, the clinician determines whether the assigned moisturizer should be applied to the subject as part of the customizable skin care regimen. In some instances, the subject determines whether to apply the assigned moisturizer as part of the customizable skin care regimen. In some embodiments, the moisturizer is a water-based moisturizer. In some embodiments, the water-based moisturizer is a lightweight cream moisturizer. In some embodiments, the water-based moisturizer is an antioxidant-containing moisturizer. In some embodiments, the water-based moisturizer is an ester-based moisturizer. In some embodiments, the water-based moisturizer is an antioxidant-containing moisturizer. In some embodiments, the water-based moisturizer is an emollient cream moisturizer. In some embodiments, the water-based moisturizer is a peptide-containing moisturizer. In some embodiments, the moisturizer comprises syringa vulgaris lilac leaf stem cell culture extract, palmitoyl tripeptide-38, and cranberry fruit extract. In some embodiments, the moisturizer comprises a blend of antioxidant compounds and peptides that function to keep skin nourished. In some embodiments, use of the moisturizer is beneficial for aging, dry, or dehydrated skin. In some embodiments, the peptide-containing moisturizer comprises the following list of ingredients: Aqua (Water), Caprylic/Capric Triglyceride, Coco-Caprylate/Caprate, Cetearyl Alcohol, Glyceryl Stearate, Glycerin, Tapioca Starch, Cetearyl Olivate, Niacinamide, Carthamus Tinctorius (Safflower) Seed Oil, Phenoxyethanol, Alcohol Denat., Sorbitan Olivate, Stearic Acid, Carbomer, Polysorbate 20, Polysorbate 60, Ethylhexylglycerin, Potassium Sorbate, Allantoin, Bisabolol, Maltodextrin, Hamamelis Viginiana (Witch Hazel) Water, Citric Acid, Sodium Hydroxide, Fructooligosaccharides (D-beta), Citrus Nobilis (Mandarin Orange) Peel Oil, Camellia Sinensis (Green Tea) Leaf Extract, Chamomilla Recutita (Matricaria) Flower Extract, Syringa Vulgaris (Lilac) Leaf Cell Culture Extract, Totarol, Glycyrrhiza Glabra (Licorice) Root Extract, Citrus Aurantium Dulcis (Orange) Peel Oil, and Vaccinium Macrocarpon (Cranberry) Fruit Extract. In some embodiments, the moisturizer is a lightweight cream moisturizer. In some embodiments, the moisturizer has a hydrating, antioxidant, and soothing effect on the skin. In some embodiments, the moisturizer comprises lilac stem cells, green tea extract, and allantoin. In some embodiments, the lightweight cream moisturizer comprises the following list of ingredients: Aqua (Water), Caprylic/Capric Triglyceride, Coco-Caprylate/Caprate, Cetearyl Alcohol, Glyceryl Stearate, Glycerin, Tapioca Starch, Cetearyl Olivate, Niacinamide, Carthamus Tinctorius (Safflower) Seed Oil, Phenoxyethanol, Alcohol Denat., Sorbitan Olivate, Stearic Acid, Carbomer, Polysorbate 20, Polysorbate 60, Ethylhexylglycerin, Potassium Sorbate, Allantoin, Bisabolol, Maltodextrin, Hamamelis Viginiana (Witch Hazel) Water, Citric Acid, Sodium Hydroxide, Fructooligosaccharides (D-beta), Citrus Nobilis (Mandarin Orange) Peel Oil, Camellia Sinensis (Green Tea) Leaf Extract, Chamomilla Recutita (Matricaria) Flower Extract, Syringa Vulgaris (Lilac) Leaf Cell Culture Extract, Totarol, Glycyrrhiza Glabra (Licorice) Root Extract, Citrus Aurantium Dulcis (Orange) Peel Oil, and Vaccinium Macrocarpon (Cranberry) Fruit Extract.

### Sun Protection

In some embodiments, systems and methods of acne clearance of customizable skin care regimens described herein comprise daily application of a sunscreen as directed by a specific customizable skin care regimen. In some embodiments, application of the sunscreen is optional. In some embodiments, the specific customizable skin care regimen is first determined at the initial assessment and a sunscreen is assigned to the subject for at-home use as part of the specific customizable skin care regimen. In some embodiments, the specific customizable skin care regime is first determined during the initial in-clinic treatment and a sunscreen is assigned to the subject for at-home use as part of the specific customizable skin care regimen. In some embodiments, the specific customizable skin care regime is modified depending on the response of the subject's facial skin during one or more in-clinic assessments and treatment that follow the initial in-clinic treatment and a sunscreen is assigned to the subject for continued or new at-home use as part of the specific customizable skin care regimen. In some instances, the clinician determines whether the assigned sunscreen should be applied to the subject as part of the customizable skin care regimen. In some instances, the subject determines whether to apply the assigned sunscreen as part of the customizable skin care regimen. In some embodiments, one of more components of the specific customizable skin care regimen renders the facial skin of the subject more susceptible to UV damage. In some embodiments, an appropriate sunscreen is used for sun protection. In some embodiments, the subject is instructed not to use a means of artificial tanning such as a tanning bed during the customizable skin care regimen. In some embodiments, the sunscreen is applied to the face of the subject following application of the advanced topical formulation of micronized benzoyl peroxide and application of any hydrator and/or moisturizer in the AM. In some embodiments, the sunscreen is a broad-spectrum sunscreen. In some embodiments, the broad-spectrum sunscreen is a non-comedogenic sunscreen. In some embodiments, the sunscreen comprises the active ingredients Zinc Oxide and Titanium Dioxide. Broad-spectrum sunscreens are effective in protecting against the harmful effects of both UVA and UVB rays. SPF rating is indicative of extent of protection from UVB rays. Some sunscreens, such as mineral-based sunscreens, comprise one or more sunscreen ingredients that act as physical blockers; scattering or reflecting UV rays away from the skin. Zinc oxide and titanium dioxide are examples of physical blockers that can be used in mineral-based sunscreens. Some sunscreens, such as chemical-based sunscreens, comprise one or more sunscreen ingredients that act as chemical absorbers; absorbing UV rays into the skin and transforming the UV rays into heat that is then released. Avobenzone, oxybenzone, octinoxate, octocrylene, homosalate, and octisalate are examples of sunscreen active ingredients in chemical-based sunscreens. Avobenzone protects against UVA and to some extent UVB rays. In some instances, homosalate aids other sunscreen ingredients in penetrating the skin. Octocrylene can be effective at absorbing both UVA and UVB rays. Oxybenzone can filter UVA rays. Octinoxate can be effective at absorbing UVB rays. Avobenzone can be effective at absorbing UV radiation, particularly UVA rays. Sunscreen ingredients effective at blocking or absorbing UVB rays can prevent damage to the outer layers of the skin, such as preventing sunburn, blistering, and skin cancer. Sunscreen ingredients effective at blocking or absorbing UVB rays can contribute to a healthy skin appearance by preventing sun damage. Sunscreen ingredients effective at blocking or absorbing UVA rays can prevent damage to the outer layers of the skin and the deeper layers of the skin. Sunscreen ingredients effective at blocking or absorbing UVA rays can prevent damage to the deeper layers of the skin associated with skin aging, wrinkling, and skin cancer. Sunscreen ingredients effective at blocking or absorbing UVA rays can contribute to a healthy skin appearance by preventing sun damage. Preventing damage to the deep skin layers by blocking UVA rays prevent premature skin aging and maintain a youthful appearance. In some embodiments, the broad-spectrum sunscreen is a zinc oxide-based sunscreen. In some embodiments, the broad-spectrum sunscreen is a mineral-based sunscreen. In some embodiments, the broad-spectrum sunscreen is an inorganic-filter sunscreen. In some embodiments, the broad-spectrum sunscreen is formulated to SPF 28. In some embodiments, the broad-spectrum sunscreen is formulated to SPF 50. In some embodiments, the broad-spectrum sunscreen is a chemical-based sunscreen. In some embodiments, the broad-spectrum sunscreen is a non-comedogenic sunscreen. In some embodiments, the non-comedogenic sunscreen is a mineral-based sunscreen. In some embodiments, the non-comedogenic sunscreen is a chemical-based sunscreen. Non-comedogenic sunscreens are formulated to provide protection from UV rays and to not block pores. In some embodiments, the broad-spectrum sunscreen is an organic filter-based sunscreen. In some embodiments, the zinc oxide-based sunscreen comprises the following list of ingredients: Active Ingredients: 11.6% Zinc Oxide, 0.67% Titanium Dioxide; Inactive Ingredients: Aqua (Water), Caprylic/Capric Triglyceride, Cetearyl Alcohol, Cetearyl Glucoside, Coco-Caprylate/Caprate, Polysorbate 60, Glycerin, Squalane, Glyceryl Isostearate, Dicaprylyl Ether, Isononyl Isononanoate, Magnesium Aluminum Silicate, 1,2-Hexanediol, Citric Acid, Caprylyl Glycol, Polyglyceryl-6 Polyricinoleate, Xanthan Gum, Polyhydroxystearic Acid, Leuconostoc/Radish Root Ferment Filtrate, Populus Tremuloides Bark Extract, Alumina, Gluconolactone, Potassium Sorbate, Aloe Barbadensis Leaf Juice Powder, Helianthus Annuus (Sunflower) Seed Oil, Trisodium Ethylenediamine Disuccinate, Lonicera Caprifolium (Honeysuckle) Flower Extract, Lonicera Japonica (Honeysuckle) Flower Extract, Fructooligosaccharides (D-beta), Allantoin, Simethicone, Tocopherol (D-alpha), and Tropolone. In some embodiments, the non-comedogenic sunscreen comprises the active ingredients Ocstisalate, Oxybenzone, Octocrylene, Homosalate and Avobenzone. In some embodiments, the non-comedogenic sunscreen comprises the active ingredients Homosalate, Octisalate, and Avobenzone. In some embodiments, the non-comedogenic sunscreen comprises the following list of ingredients: Active Ingredients: Homosalate about 10.0%, Octisalate about 5.0%, Avobenzone about 3.0%; Inactive Ingredients: Aqua (Water), Butyloctyl Salicylate, Glyceryl Stearate, Diethylhexyl 2,6-Naphthalate, C12-15 Alkyl Benzoate, Dimethicone, Glycerin, Hamamelis Virginiana (Witch Hazel) Bark/Twig Extract, Niacinamide, Panthenol, Bisabolol, Cetearyl Alcohol, Tocopheryl Acetate, Sodium Hyaluronate, Potassium Azeloyl Diglycinate, Allantoin, Potassium Cetyl Phosphate, Polymethylsilsesquioxane, Bis-Dodecylhexadecyl Dimer Dilinoleate/Propanediol Copolymer, Phenoxyethanol, Propylheptyl Caprylate, Carbomer, Ethylhexylglycerin, Sodium Phytate, and Sodium Hydroxide. In some embodiments, the chemical-based sunscreen comprises the following list of ingredients: Active Ingredients: Octisalate 4.75% (Sunscreen), Oxybenzone 4.25% (Sunscreen), Octocrylene 2.80% (Sunscreen), Avobenzone 3.00% (Sunscreen); Inactive Ingredients: Acrylates/C12-22 Alkylmethacrylate Copolymer, Alerurites Moluccanus Seed (Kukui Nut Oil), Allantoin, Butyloctyl Salicylate, Benzoic Acid, C12-C15 Alkyl Benzoate, Camellia Sinensis (Green Tea) Leaf Extract, Cetearyl Alcohol, Cyclopentasiloxane, Dimethicone, Ethylhexylglycerin, Glycereth-2 Cocoate, Glycerin, Helianthus Annuus (Sunflower) Seed Oil, Macadamina (Nut) Ternifolia Seed Oil, Magnesium Aluminum Silicate, Phenoxyethanol, Polyacrylate Crosspolymer-6, Potassium Cetyl Phosphate, Tetrasodium EDTA, Tocopheryl Acetate (Vitamin E Acetate), Tocopherol, and Water (Aqua). In some embodiments, the non-comedogenic sunscreen has an SPF of at least about 50. In some embodiments, the sunscreen is a combination mineral and chemical-based sunscreen. In some embodiments, the combination mineral and chemical-based sunscreen is a hybrid organic and inorganic filter-based sunscreen. In some embodiments, the combination mineral and chemical-based sunscreen is rated at about SPF 15, about SPF 20, about SPF 25, about SPF 30, about SPF 35, about SPF 40, about SPF 45, or about SPF 50. In some embodiments, the non-comedogenic sunscreen is rated to at least about SPF 15, at least about SPF 20, at least about SPF 25, at least about SPF 30, at least about SPF 35, at least about SPF 40, at least about SPF 45, or at least about SPF 50. In some embodiments, the customizable skin care regimen to the subject comprises applying a non-comedogenic sunscreen to the skin of the subject daily in the AM.

### Topical Acne-Clearing Creams

Systems and methods of acne clearance of customizable skin care regimens described herein comprise daily application of one or more topical acne-clearing creams as directed by a specific customizable skin care regimen. In some embodiments, the system of acne clearance uses one or more advanced acne medication formulations. In some embodiments, the advanced acne medication formulation is an advanced topical formulation. In some embodiments, the advanced topical formulation is a benzoyl peroxide-based acne treatment topical formulation. In some embodiments, the advanced topical formulation is an advanced formulation of 2.5% micronized benzoyl peroxide. In some embodiments, the advanced topical formulation is an advanced formulation of 5% micronized benzoyl peroxide. In some embodiments, the advanced topical formulation is an advanced formulation of 10% micronized benzoyl peroxide. In some embodiments, the specific customizable skin care regime is first determined at the initial assessment and one or more topical acne-clearing creams are assigned to the subject for at-home use as part of the specific customizable skin care regimen. In some embodiments, the specific customizable skin care regimen is first determined during the initial in-clinic treatment and one or more topical acne-clearing creams are assigned to the subject for at-home use as part of the specific customizable skin care regimen. In some embodiments, the specific customizable skin care regimen is modified depending on the response of the subject's facial skin during one or more in-clinic assessments and treatment that follow the initial in-clinic treatment and one or more topical acne-clearing creams are assigned to the subject for continued or new at-home use as part of the specific customizable skin care regimen. In some embodiments, the topical acne-clearing cream is an advanced topical formulation of micronized benzoyl peroxide. Micronized suspensions can provide effective delivery of small particles of benzoyl peroxide to the skin upon topical application. Micronized suspensions are suspensions containing micronized benzoyl peroxide and are preferred to standard or non-micronized suspensions of benzoyl peroxide. In some embodiments, the one or more topical acne-clearing creams function as anti-acne formulations. In some embodiments, the advanced topical formulation micronized benzoyl peroxide functions to reduce the number of acne-causing bacteria on the face of the subject and within the pores. In some embodiments, the advanced topical formulation micronized benzoyl peroxide functions to reduce rosacea. In some instances, skin reactions including peeling, itching, irritation, and reddening of the skin may occur after the initiation of daily application of the advanced topical formulation of micronized benzoyl peroxide. In some instances, the specific customizable skin care regimen is adjusted based on the response of the subject in effectively clearing acne and the selected advanced topical formulation of micronized benzoyl peroxide for use by the subject is adjusted. In some embodiments, the advanced topical formulation of micronized benzoyl peroxide comprises about 2.5% micronized benzoyl peroxide w/v. In some embodiments, the advanced topical formulation of micronized benzoyl peroxide comprises about 5% micronized benzoyl peroxide w/v. In some embodiments, the advanced topical formulation of micronized benzoyl peroxide comprises about 10% micronized benzoyl peroxide w/v. In some embodiments, the advanced topical formulation micronized benzoyl peroxide is termed either 2.5% Advanced Acne Med, 5% Advanced Acne Med, or 10% Advanced Acne Med. In some embodiments, the advanced formulation of 2.5% micronized benzoyl peroxide comprises the following list of ingredients: Active Ingredient: Benzoyl Peroxide 2.5%; Inactive Ingredients: Water (Aqua), Glycerin, Propanediol, Sodium Hyaluronate, Bisabolol, Carbomer, Ammonium Acryloyldimethyltaurate/VP Copolymer, Xanthan Gum, Sodium Hydroxide, Phenoxyethanol, and Ethylhexylglycerin. In some embodiments, the advanced formulation of 5% micronized benzoyl peroxide comprises the following list of ingredients: Active Ingredient: Benzoyl Peroxide 5%; Inactive Ingredients: Water (Aqua), Glycerin, Propanediol, Sodium Hyaluronate, Bisabolol, Carbomer, Ammonium Acryloyldimethyltaurate/VP Copolymer, Xanthan Gum, Sodium Hydroxide, Phenoxyethanol, and Ethylhexylglycerin. In some embodiments, the advanced formulation of 10% micronized benzoyl peroxide comprises the following list of ingredients: Active Ingredient: Benzoyl Peroxide 10%; Inactive Ingredients: Water (Aqua), Glycerin, Propanediol, Sodium Hyaluronate, Bisabolol, Carbomer, Ammonium Acryloyldimethyltaurate/VP Copolymer, Xanthan Gum, Sodium Hydroxide, Phenoxyethanol, and Ethylhexylglycerin. In some embodiments, the advanced acne medication formulation effectively treats acne. In some embodiments, the advanced acne medication formulation effectively clears acne. In some embodiments, the advanced acne medication formulation penetrates pores to control acne blemishes, blackheads, and whiteheads. In some embodiments, the advanced acne medication formulation prevents new acne blemishes from forming. In some embodiments, the advanced acne medication formulation applies smoothly and penetrates more deeply and consistently compared to non-micronized benzoyl peroxide topical formulations making it a more effective in acne clearance. In some embodiments, the advanced acne medication formulation components bisabolol and sodium hyaluronate function to minimize irritation. In some embodiments, the advanced acne medication formulation components bisabolol and sodium hyaluronate function to minimize excessive dryness due to benzoyl peroxide. In some embodiments, the advanced acne medication formulation is applied after cleansing the skin, and following application of toner, serum, and optional hydrator. In some embodiments, the advanced topical formulation of 5% micronized benzoyl peroxide is used to increase from the advanced topical formulation of 2.5% micronized benzoyl peroxide due to insufficient acne clearance. In some embodiments, the advanced topical formulation of 10% micronized benzoyl peroxide is used to increase from the advanced topical formulation of 5% micronized benzoyl peroxide due to insufficient acne clearance. In some embodiments, the advanced topical formulation of micronized benzoyl peroxide is used in subjects seeking improved skin appearance and clearance of acne wherein the subject has either a normal or an oily skin type.

In some embodiments, an optional sulfur spot treatment is applied to the face of subject in one or more areas of inflamed acne as the second of the one or more topical acne-clearing creams. Use of the sulfur spot treatment is indicated for use in mild, moderate, or severe inflamed or combination acne. Use of the sulfur spot treatment is not indicated for use in moderate or severe noninflamed acne. In some embodiments, the sulfur spot treatment comprises the following list of ingredients: Active Ingredient: Sulfur 6%; Inactive Ingredients: Aqua (Water), Butylene Glycol, Acacia Senegal Gum, Carbomer, Phenoxyethanol, Bisabolol, Allantoin, Benzyl Alcohol, Glycerin, Caprylhydroxamic Acid, Dextran, Sodium Hydroxide, Ethylhexylglycerin, and Palmitoyl Tripeptide-8. In some embodiments, the sulfur spot treatment is invisible following application. In some embodiments, the sulfur spot treatment comprises a beneficial peptide that improves red and inflamed acne lesions. In some embodiments, the sulfur spot treatment is applied in the AM, in the PM, or at any time throughout the day. In some embodiments, the sulfur spot treatment provides a soothing effect to the skin of the subject. In some embodiments, a small amount (e.g., pea-sized) of the sulfur spot treatment is applied to areas of inflamed acne breakouts after application of serum, optional hydrator, and advanced topical formulation of micronized benzoyl peroxide. Following application of the sulfur spot treatment, the subject is directed to allow at least 1-2 minutes for the spot treatment to dry and then follow up with the customizable skin care regimen to apply the optional moisturizer and the sunscreen.

### Professionally Administered Acid-Based Facial Chemical Peels

Systems and methods of acne clearance of customizable skin care regimens described herein comprise one or more professionally administered acid-based facial chemical peels following either an initial assessment or an in-clinic treatment. In some embodiments of methods for clearing inflamed or combination acne in a subject in need thereof described herein, the method comprises conducting an in-clinic assessment and treatment comprising at least one facial acne peel or an acne hydrating treatment, wherein the assessment and treatment are each administered by a clinician. In some embodiments, the in-clinic assessment and treatment begin on or around the same starting date as the customizable skin care regimen begins, and the in-clinic assessment and treatment are recurrently conducted by the clinician until the subject has achieved a significant extent of clearance of inflamed acne or combination acne. In some embodiments, the subject has mild inflamed or combination acne at the time of a first in-clinic assessment. In some embodiments, the subject has moderate inflamed or combination acne at the time of a first in-clinic assessment. In some embodiments, the subject has severe inflamed or combination acne at the time of a first in-clinic assessment. In some embodiments, the in-clinic assessment and treatment are used to revise the customizable skin care regimen to: i) achieve favorable acne clearance and improved facial skin condition in the subject. In some embodiments, the in-clinic assessment and treatment are used to revise the customizable skin care regimen to: ii) improve tolerance to skin care products selected as part of the customizable skin care regimen. In some embodiments, the in-clinic assessment and treatment are used to revise the customizable skin care regimen to: iii) achieve favorable acne clearance and improved facial skin condition in the subject and improve tolerance to skin care products selected as part of the customizable skin care regimen. In some embodiments of methods for clearing moderate or severe noninflamed acne in a subject in need thereof described herein, the method comprises conducting an in-clinic assessment and treatment comprising at least one facial acne peel or an acne hydrating treatment, wherein the assessment and treatment are each administered by a clinician. In some embodiments, the in-clinic assessment and treatment begin on or around the same starting date as the customizable skin care regimen begins, and the in-clinic assessment and treatment are recurrently conducted by the clinician until the subject has achieved a significant extent of clearance of noninflamed acne. In some embodiments, the subject has moderate noninflamed acne at the time of a first in-clinic assessment, and the subject does not have inflamed acne at the time of the first in-clinic assessment. In some embodiments, the subject has severe noninflamed acne at the time of a first in-clinic assessment, and the subject does not have inflamed acne at the time of the first in-clinic assessment. In some embodiments, the in-clinic assessment and treatment are used to revise the customizable skin care regimen to: i) achieve favorable acne clearance. In some embodiments, the in-clinic assessment and treatment are used to revise the customizable skin care regimen to: ii) improve facial skin condition in the subject. In some embodiments, the in-clinic assessment and treatment are used to revise the customizable skin care regimen to: iii) improve tolerance to skin care products selected as part of the customizable skin care regimen. In some embodiments, the in-clinic assessment and treatment are used to revise the customizable skin care regimen to: i) achieve favorable acne clearance; ii) improve facial skin condition in the subject; iii) improve tolerance to skin care products selected as part of the customizable skin care regimen, or any combination thereof. In-clinic treatments are administered at the initial in-clinic treatment and may be administered at Week 1, Week 3, Week 5, Week 7, Week 9, Week 11, Week 13, Week 15, and any subsequent period of two weeks in which the subject maintains participation in the customizable skin care regimen. At each assessment, the clinician grades the facial acne severity of the subject and provides any needed revisions to the customizable skin care regimen to improve acne clearing, tolerance, and appearance. At each assessment, including at the initial assessment, the clinician records a total inflamed acne count and total noninflamed acne count. At each assessment following the initial assessment, the clinician compares the recorded total inflamed acne count and total noninflamed acne count to the total inflamed acne count and total noninflamed acne count from the last assessment or from the initial assessment. In some embodiments, statistical analysis (e.g., paired t-tests, repeated measures of analysis of variance [ANOVA], or a regression model, etc.) is used to determine if the subject has achieved a significant extent of clearance of inflamed acne or combination acne or a significant extent of clearance of noninflamed acne, or a significant extent of clearance of inflamed acne or combination acne and a significant extent of clearance of noninflamed acne. In some embodiments, a reduction in IGA acne severity score is used to determine if the subject has achieved a significant extent of clearance. In some embodiments, the subject achieves a significant clearance in acne as defined by having an IGA acne severity score of 0 or 1. In some embodiments, the subject achieves a significant clearance in acne as defined by having an IGA acne severity score of 0 or 1, with at least a two-grade improvement from baseline. In some embodiments, the subject achieves a significant clearance in acne as measured by moving from IGA acne severity score 4 to 1. In some embodiments, the subject achieves a significant clearance in acne as measured by moving from IGA acne severity score 4 to 0. In some embodiments, the subject achieves a significant clearance in acne as measured by moving from IGA acne severity score 3 to 1. In some embodiments, the subject achieves a significant clearance in acne as measured by moving from IGA acne severity score 3 to 0. In some embodiments, the subject achieves a significant clearance in acne as measured by moving from IGA acne severity score 2 to 0. In some embodiments, the method achieves favorable acne clearance and improved facial skin condition in the subject. In some embodiments, favorable acne clearance is a significant extent of clearance. In some embodiments, the method achieves improved tolerance to skin care products selected as part of the customizable skin care regimen. In some embodiments, the method achieves favorable acne clearance, improved facial skin condition in the subject, and improved tolerance to skin care products selected as part of the customizable skin care regimen. In some embodiments, the method achieves favorable acne clearance and improved facial skin condition in the subject. In some embodiments, the method achieves favorable acne clearance and improved tolerance to skin care products selected as part of the customizable skin care regimen. In some embodiments, the initial professionally administered acid-based facial chemical peel is conducted at Week 1. In some embodiments, acid-based facial chemical peel 1 is conducted at Week 1, Week 3, and Week 5. In some embodiments, acid-based facial chemical peel 2 is conducted at Week 7, Week 9, Week 11, Week 13, Week 15, and any subsequent two-week period. In some embodiments, acid-based facial chemical peel 3 is conducted at Week 11, Week 13, Week 15, and any subsequent two-week period. In some embodiments, if the clinician determines severity of 0, 1, or 2 on a scale of 0-10 of tingling, stinging, and/or burning in a skin tolerance test with use of the glycolic-lactic toner, acid-based facial chemical peel 1, acid-based facial chemical peel 2, or acid-based facial chemical peel 3 is selected by the clinician for the subject. In some embodiments, application of the exfoliating toner is required prior to conducting the facial acne peel. In some embodiments, if the clinician determines severity of 3 or greater on a scale of 0-10 of tingling, stinging, and/or burning in a skin tolerance test with use of the exfoliating toner, Acne Hydrating Treatment is selected by the clinician for the subject. In some embodiments, Acne Hydrating Treatment is conducted at Week 1, Week 3, Week 5, Week 7, Week 9, Week 11, Week 13, Week 15, and any subsequent two-week period. In some embodiments, the facial acne peel is used to clear acne by exfoliating the skin, unclogging pores, and reducing inflammation. In some embodiments, the facial acne peel improves skin textures. In some embodiments, the facial acne peel reduces the appearance of acne scars. In some embodiments, the facial acne peel reduces the appearance of large pores. In some embodiments, the facial acne peel fades areas of hyperpigmentation. In some embodiments, the facial acne peel dries areas of acne breakouts. In some embodiments, the facial acne peel is administered by the clinician using a brush, cotton ball, gauze, spatula, or sponge to apply the acne peel solution to the skin. For light and medium peels, the forehead and temples of the subject are treated first, followed by the cheeks and chin, and finally, optionally, the areas around the eyes and mouth. The strength of the peel determines sensation while the solution is on the skin. Acid-based facial peel levels rated as 1, 2, or 3 peels typically do not require anesthesia or pain medication. In some embodiments, the solution is not removed from the skin and the solution is self-neutralizing. Following the peel procedure, the skin of the subject is cleansed, and sunscreen is applied. In some embodiments, acid-based chemical peel 1 is a trichloroacetic acid (TCA)-based peel. In some embodiments, acid-based chemical peel 1 is a TCA and lactic acid-based peel. In some embodiments, acid-based chemical peel 1, acid-based chemical peel 2, and acid-based chemical peel 3 are acid-based peels which may include TCA, alpha hydroxy acids, salicylic acid, or kojic acid, or any combination thereof. In some embodiments, acid-based chemical peel 1 is a superficial acid-based chemical peel. In some embodiments, acid-based chemical peel 2 and acid-based chemical peel 3 is a TCA and mandelic acid-based peel. In some embodiments, the peel is a non-TCA peel. In some embodiments, non-TCA peel is a chemical peel containing 15-40% acid (which may include alpha hydroxy acids, salicylic acid, enzymes, and kojic acid). In some embodiments, the solution used for acid-based chemical peel 1 comprises the following list of ingredients: Aqua (Water), Alcohol Denat., Glycerin, Mandelic Acid (L), Trichloroacetic Acid, Lactic Acid (L), Phenoxyethanol, Kojic Acid, Sodium Lactate, Benzyl Alcohol, Sodium PCA, Potassium Azeloyl Diglycinate, Sodium Gluconate, Caprylhydroxamic Acid, Sodium Hyaluronate (L), Ethylhexylglycerin, Sorbitol, Proline (L), Gluconolactone, Sodium Benzoate, and Diglucosyl Gallic Acid. In some embodiments, acid-based chemical peel 2 is a TCA and mandelic acid-based peel. In some embodiments, the solution used for acid-based chemical peel 2 comprises the following list of ingredients: Aqua (Water), Alcohol Denat., Glycerin, Trichloroacetic Acid, Mandelic Acid (L), Lactic Acid (L), Phenoxyethanol, Kojic Acid, Benzyl Alcohol, Sodium Lactate, Potassium Azeloyl Diglycinate, Sodium Gluconate, Caprylhydroxamic Acid, Sodium PCA, Sodium Hyaluronate (L), Ethylhexylglycerin, Sorbitol, Proline (L), Gluconolactone, Sodium Benzoate, and Diglucosyl Gallic Acid. In some embodiments, the solution used for acid-based chemical peel 3 comprises the following list of ingredients: Aqua (Water), Alcohol Denat., Trichloroacetic Acid, Glycerin, Mandelic Acid (L), Lactic Acid (L), Phenoxyethanol, Kojic Acid, Sodium Lactate, Benzyl Alcohol, Sodium PCA, Potassium Azeloyl Diglycinate, Sodium Gluconate, Caprylhydroxamic Acid, Ethylhexylglycerin, Sorbitol, Proline (L), Gluconolactone, Sodium Benzoate, Diglucosyl Gallic Acid, and Sodium Hyaluronate (L). In some embodiments, the solution used for the Hydrating Enzyme Mask comprises the following list of ingredients: Aqua (Water), Caprylic/Capric Triglyceride, Squalane, Glycerin, Hydroxyethyl Acrylate/Sodium Acryloyldimethyl Taurate Copolymer, Cetearyl Alcohol, Behenyl Alcohol, Carnitine (L), Sodium Acrylate/Acryloyldimethyltaurate/Dimethylacrylamide Crosspolymer, Phenoxyethanol, Coco-Glucoside, Isohexadecane, Benzyl Alcohol, Sodium PCA, Sodium Gluconate, Caprylhydroxamic Acid, Polysorbate 60, Allantoin, Bisabolol, Carica Papaya (Papaya) Fruit Extract, Sodium Hyaluronate (L), Ethylhexylglycerin, Papain, Caprylyl Glycol, and Acetyl Heptapeptide-4. In some embodiments, the peel is an enzyme-based hydrating mask. In some embodiments, the peel is an enzyme-based chemical peel. In some embodiments, the enzyme-based chemical peel is a papain-containing hydrating mask.

### Subjects

Compositions and formulations described herein may be used as part of a skin care regimen that comprises topical application of the composition or formulation. The skin care regimen may be designed to improve or clear acne in the subject. The subject may be assessed to determine their Fitzpatrick skin type complexion to inform parameters of the skin care regimen. The subject may be assessed to determine their skin type based on the American Academy of Dermatology system of classification containing five main skin types of: 1) normal, 2) dry, 3) oily, 4) combination, or 5) sensitive, to inform parameters of the skin care regimen. The subject may be assessed for a dermatological condition with an extent of acne according to the Investigator's Global Assessment (IGA) of acne severity to inform parameters of the skin care regimen. The following are the guidelines according to IGA of acne severity:
Level 0, ‘Clear’, Residual hyperpigmentation and erythema may be present.
Level 1, ‘Almost clear’, A few scattered comedones and a few small papules are present.
Level 2, ‘Mild’, Acne is easily recognizable; less than half the face is involved. Some comedones and some papules and pustules are present.
Level 3, ‘Moderate’, More than half the face is involved. Many comedones, papules and pustules are present. One nodule may be present.
Level 4, ‘Severe’, Entire face is involved, covered with comedones, numerous papules and pustules are present, and few nodules and cysts are present.

In some embodiments of methods for clearing acne described herein, the method comprises assessing an extent of acne severity in the subject. In some embodiments, the extent of acne severity in the subject is assessed by completing a count of inflamed and noninflamed facial acne in the subject. In some embodiments, improvement in an extent of acne severity in the subject is determined by comparing a count of inflamed and noninflamed facial acne in the subject to a previously taken count of inflamed and noninflamed facial acne in the subject. In some embodiments, the extent of acne severity in the subject is assessed according to the Investigator's Global Assessment (IGA) of acne severity. In some embodiments, improvement in an extent of acne severity in the subject is determined by comparing an IGA acne severity score in the subject to a previous IGA acne severity score in the subject. In some embodiments, the previous IGA acne severity score in the subject was determined at an initial assessment prior to the start of a customized skin care regimen. In some embodiments, the previous IGA acne severity score in the subject was determined during a previous in-clinic treatment. In some embodiments of systems for clearing acne described herein, the system is configured to perform the method steps of a customized skin care regimen. In some embodiments, the method steps of a customized skin care regimen comprise assessing an extent of acne severity in a subject, wherein the subject is in need of clearing inflamed acne or combination acne, or wherein the subject is in need of clearing noninflamed acne.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,”, “participant”, or “patient” are often used interchangeably herein. A “subject” can be a biological entity to whom the composition or formulation is applied. subject can be a human. The subject may be diagnosed or observed to have a dermatological condition. The dermatological condition may include acne vulgaris.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

The section headings used herein are for organizational purposes only and are not to be construed as limiting the subject matter described.

### EXAMPLES

The following examples are included for illustrative purposes only and are not intended to limit the scope of the inventive concepts.

### Example 1: Quantitative Advanced Formulation of 2.5% Micronized Benzoyl Peroxide for Use with Acne Home Care Protocol

In this example, an advanced acne medication formula is presented for use in home skin care protocol for acne improvement and clearance. This advanced acne medication formula is for use as a topical bactericidal-based acne treatment formulation within systems and methods for acne clearance. Key ingredients of the formulation include about 2.5% micronized benzoyl peroxide, bisabolol, and sodium hyaluronate. Micronized benzoyl peroxide is utilized for a smaller and more consistent benzoyl peroxide particle size that penetrates skin pores more efficiently and has a smoother, less gritty consistency than non-micronized benzoyl peroxide. This micronized benzoyl peroxide also prevents potential skin irritation following topical application to the face of a subject in need thereof. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The smoother consistency and the less potential for dryness and/or irritation contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness. The Acne Medication 2.5% BPO formulation described in this Example comprises three components that function as rheology modifiers to determine favorable consistency and effective delivery of the anti-acne active ingredient (micronized BPO). The selected rheology modifiers are Xanthan Gum, Carbomer, and Ammonium Acryloyldimethyltaurate/VP Copolymer. The concentration of each of the listed rheology modifiers influences the flow of the formulation to facial skin following topical application. Flow of the formulation may be measured using shear and the shear parameters of stress (τ) and strain rate (γ′) are calculated from measurements of torque and flow rate. The rheology modifiers and concentrations thereof were selected to create a rheology profile for precise delivery of the micronized BPO. The rheology profile for the formulation includes the yield stress and the viscosity at different shear modes and rates. The rheology profile of the formulation provides a functional formulation network, provides an easy to apply structure in viscosity, and generates favorable glide and suspension properties. The basic rheology-determining parameters of an emulsion or lotion are considered a continuous phase rheology and determine the nature of the drops (size distribution, deformability, internal viscosity, concentration of particles and particles interactions). In this Example, the selected rheology modifiers help to suspend micronized benzoyl peroxide particles evenly throughout the formulation to prevent any settling or sedimentation and help with uniform distribution and effective delivery. This prevention of settling of micronized benzoyl peroxide particles ensures consistency throughout a packaged formulation. The selected rheology modifiers control viscosity and flow to improve consistency of application of the formulation and extend useable lifetimes of a packaged formulation. Besides just viscosity, the structure and solid-like properties of micro dispersions play a crucial role in dispersion attributes and behaviors of micronized benzoyl peroxide particles. The rheology profile of the formulation provides a system that holds active ingredient BPO and helps with suspending, delivery, and uniform distribution. Xanthan gum is a rheology control agent that improves the texture, stability, and appearance of the formulation. Xanthan gum also is a naturally occurring polysaccharide that is nontoxic and nonirritating. Xanthan Gum concentration makes the formulation feel smoother and silkier upon topical application and can help reduce sensory irritation in the subject by forming a protective barrier on the skin. Carbomer primarily functions as a rheology modifier, meaning it controls the consistency and flow of the formulation by significantly increasing its viscosity, acting as a thickener to create gel form with a smooth, spreadable texture while maintaining stability and preventing separation of ingredients. This allows for even application and improved delivery of active ingredients to the skin upon topical application. By controlling viscosity, carbomer contributes to a desirable texture and feel on the skin, making the formulation easier to spread and apply. By controlling viscosity, carbomer contributes to a desirable texture and feel on the skin, making the formulation easier to spread and apply and creates an easy water breaking sensation that helps transferring active ingredient. Ammonium Acryloyldimethyltaurate/VP Copolymer forms a cross-linked polymer network that traps particles of micronized BPO, creating a stable structure and keeping the BPO particles in the form and sizes that can penetrate deeper into the acne pores. It also works as thickening and gelling agent plus as texturizer and provides a light, fresh, and non-tacky skin feel. The rheology profile of a topical formulation with micronized benzoyl peroxide generates enhanced characteristics by significantly improving an ability of the micronized benzoyl peroxide particles to spread and penetrate the skin due to their smaller micronized particle size, thereby allowing for better distribution of the BPO within the formulation. This leads to increased efficacy and reduced irritation compared to standard benzoyl peroxide particles; making the formulation smoother and easier to apply while enhancing its penetration into the skin's pores. By distributing the active ingredient of micronized benzoyl peroxide more evenly, rheology modifiers reduce irritation caused by concentrated clusters of large particles.

### Contraindications

This formulation is not to be provided to subjects that are allergic to benzoyl peroxide.

The components and respective concentrations in the formulation are listed in Table 1 below. As provided in Table 1, the International Nomenclature of Cosmetic Ingredients (INCI)/Common Name is provided for each component.

TABLE 1
Formula A-Acne Medication 2.5% BPO

| INCI/Common Name | % w/w |
|---|---|
| Benzoyl Peroxide | 2.5-2.625 |
| Glycerin | 2.991 |
| Phenoxyethanol | 0.900 |
| Ethylhexylglycerin | 0.100 |
| Propanediol | 1.000 |
| Sodium Hydroxide | 0.160 |
| Carbomer | 0.400 |
| Ammonium | 0.300 |
| Acryloyldimethyltaurate/VP | |
| Copolymer | |
| Xanthan Gum | 0.100 |
| Sodium Hyaluronate | 0.01-0.1 |
| Bisabolol | 0.010-0.10 |
| Aqua | QS 100 |

### Subject Information

In some instances, the subject is assessed to determine their skin type (e.g., normal, dry, oily, combination, sensitive, or the like), acne type and acne severity, Fitzpatrick skin complexion type, or other skin conditions and concerns to inform parameters of the skin care regimen, or any combination thereof. The formulation used and frequency of use may vary with the skin assessment of the subject, the age and physical condition of the subject, the duration of the treatment, the response and tolerability by the subject, and like factors. In some instances, the subject is a mammal. In some instances, the subject is a human. In some instances, the subject is a male. In some instances, the subject is a female. In some instances, the human subject is at least about 18 years of age. In some instances, the human subject is a child or adult 12 years of age or over. In some instances, the human subject is an adult between the ages of 18-79.

Recommendations during use of formulation to improve or clear acne: subjects are encouraged to stay sufficiently hydrated during ongoing skin care treatment regimens using this formulation.

### Usage and Tips:

The subject should apply a dime to nickel size amount to entire area, avoid neck and eyes. Let dry. The topical formulation should be applied all over the affected area; do not spot treat. Subject is recommended to apply a hydrating gel underneath the advanced acne medication formulation for added hydration.

### Example 2: Quantitative Advanced Formulation of 5% Micronized Benzoyl Peroxide for Use with Acne Home Care Protocol

In this example, an advanced acne medication formula is presented for use in home skin care protocol for acne improvement and clearance. This advanced acne medication formula is for use as a topical bactericidal-based acne treatment formulation within systems and methods for acne clearance. Key ingredients of the formulation include about 5% micronized benzoyl peroxide, bisabolol, and sodium hyaluronate. Micronized benzoyl peroxide is utilized for a smaller and more consistent benzoyl peroxide particle size that penetrates skin pores more efficiently and has a smoother, less gritty consistency than non-micronized benzoyl peroxide. This micronized benzoyl peroxide also prevents potential skin irritation following topical application to the face of a subject needing acne clearance. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The less potential for dryness and/or irritation and smoother consistency contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness. The Acne Medication 5% BPO formulation described in this Example comprises three components that function as rheology modifiers to determine favorable consistency and effective delivery of the anti-acne active ingredient (micronized BPO). The selected rheology modifiers are Xanthan Gum, Carbomer, and Ammonium Acryloyldimethyltaurate/VP Copolymer. The concentration of each of the listed rheology modifiers influences the flow of the formulation to facial skin following topical application. Flow of the formulation may be measured using shear and the shear parameters of stress (τ) and strain rate (γ′) are calculated from measurements of torque and flow rate. The rheology modifiers and concentrations thereof were selected to create a rheology profile for precise delivery of the micronized BPO. The rheology profile for the formulation includes the yield stress and the viscosity at different shear modes and rates. The rheology profile of the formulation provides a functional formulation network, provides an easy to apply structure in viscosity, and generates favorable glide and suspension properties. The basic rheology-determining parameters of an emulsion or lotion are considered a continuous phase rheology and determine the nature of the drops (size distribution, deformability, internal viscosity, concentration of particles and particles interactions). In this Example, the selected rheology modifiers help to suspend micronized benzoyl peroxide particles evenly throughout the formulation to prevent any settling or sedimentation and help with uniform distribution and effective delivery. This prevention of settling of micronized benzoyl peroxide particles ensures consistency throughout a packaged formulation. The selected rheology modifiers control viscosity and flow to improve consistency of application of the formulation and extend useable lifetimes of a packaged formulation. Besides just viscosity, the structure and solid-like properties of micro dispersions play a crucial role in dispersion attributes and behaviors of micronized benzoyl peroxide particles. The rheology profile of the formulation provides a system that holds active ingredient BPO and helps with suspending, delivery, and uniform distribution. Xanthan gum is a rheology control agent that improves the texture, stability, and appearance of the formulation. Xanthan gum also is a naturally occurring polysaccharide that is nontoxic and nonirritating. Xanthan Gum concentration makes the formulation feel smoother and silkier upon topical application and can help reduce sensory irritation in the subject by forming a protective barrier on the skin. Carbomer primarily functions as a rheology modifier, meaning it controls the consistency and flow of the formulation by significantly increasing its viscosity, acting as a thickener to create gel form with a smooth, spreadable texture while maintaining stability and preventing separation of ingredients. This allows for even application and improved delivery of active ingredients to the skin upon topical application. By controlling viscosity, carbomer contributes to a desirable texture and feel on the skin, making the formulation easier to spread and apply. By controlling viscosity, carbomer contributes to a desirable texture and feel on the skin, making the formulation easier to spread and apply and creates an easy water breaking sensation that helps transferring active ingredient. Ammonium Acryloyldimethyltaurate/VP Copolymer forms a cross-linked polymer network that traps particles of micronized BPO, creating a stable structure and keeping the BPO particles in the form and sizes that can penetrate deeper into the acne pores. It also works as thickening and gelling agent plus as texturizer and provides a light, fresh, and non-tacky skin feel. The rheology profile of a topical formulation with micronized benzoyl peroxide generates enhanced characteristics by significantly improving an ability of the micronized benzoyl peroxide particles to spread and penetrate the skin due to their smaller micronized particle size, thereby allowing for better distribution of the BPO within the formulation. This leads to increased efficacy and reduced irritation compared to standard benzoyl peroxide particles; making the formulation smoother and easier to apply while enhancing its penetration into the skin's pores. By distributing the active ingredient of micronized benzoyl peroxide more evenly, rheology modifiers reduce irritation caused by concentrated clusters of large particles.

### Contraindications

This formulation is not to be provided to subjects that are allergic to benzoyl peroxide.

### Subject Information

In some instances, the subject is assessed to determine their skin type (e.g., normal, dry, oily, combination, sensitive, or the like), acne type and acne severity, Fitzpatrick skin complexion type, or other skin conditions and concerns to inform parameters of the skin care regimen, or any combination thereof. The formulation used and frequency of use may vary with the skin assessment of the subject, the age and physical condition of the subject, the duration of the treatment, the response and tolerability by the subject, and like factors. In some instances, the subject is a mammal. In some instances, the subject is a human. In some instances, the subject is a male. In some instances, the subject is a female. In some instances, the human subject is at least about 18 years of age. In some instances, the human subject is a child or adult 12 years of age or over. In some instances, the human subject is an adult between the ages of 18-79. Age of subjects: young adolescents or adults.

Recommendations during use of formulation to improve or clear acne: subjects are encouraged to stay sufficiently hydrated during ongoing skin care treatment regimens using this formulation.

### Usage and Tips:

The subject should apply a dime to nickel size amount to entire area, avoid neck and eyes. Let dry. The topical formulation should be applied all over the affected area; do not spot treat. Subject is recommended to apply a hydrating gel underneath the advanced acne medication formulation for added hydration.

The components and respective concentrations in the formulation are listed in Table 2 below. As provided in Table 2, the International Nomenclature of Cosmetic Ingredients (INCI)/Common Name is provided for each component.

TABLE 2
Formula B-Acne Medication 5% BPO

| INCI/Common Name | % w/w |
|---|---|
| Benzovl Peroxide | 5.0-5.25 |
| Glycerin | 2.991 |
| Phenoxyethanol | 0.900 |
| Ethylhexylglycerin | 0.100 |
| Propanediol | 1.000 |
| Sodium Hydroxide | 0.100 |
| Carbomer | 0.400 |
| Ammonium | 0.400 |
| Acryloyldimethyltaurate/VP | |
| Copolymer | |
| Xanthan Gum | 0.200 |
| Sodium Hyaluronate | 0.01-0.1 |
| Bisabolol | 0.010-0.10 |
| Aqua | QS 100 |

### Example 3: Quantitative Advanced Formulation of 10% Micronized Benzoyl Peroxide for Use with Acne Home Care Protocol

In this example, an advanced acne medication formula is presented for use in home skin care protocol for acne improvement and clearance. This advanced acne medication formula is for use as a topical bactericidal-based acne treatment formulation within systems and methods for acne clearance. Key ingredients of the formulation include about 10% micronized benzoyl peroxide, bisabolol, and sodium hyaluronate. Micronized benzoyl peroxide is utilized for a smaller and more consistent benzoyl peroxide particle size that penetrates skin pores more efficiently than non-micronized benzoyl peroxide. This micronized benzoyl peroxide also prevents potential skin irritation following topical application to the face of a subject needing acne clearance. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The less potential for dryness and/or irritation contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness. The Acne Medication 10% BPO formulation described in this Example comprises three components that function as rheology modifiers to determine favorable consistency and effective delivery of the anti-acne active ingredient (micronized BPO). The selected rheology modifiers are Xanthan Gum, Carbomer, and Ammonium Acryloyldimethyltaurate/VP Copolymer. The concentration of each of the listed rheology modifiers influences the flow of the formulation to facial skin following topical application. Flow of the formulation may be measured using shear and the shear parameters of stress (τ) and strain rate (γ′) are calculated from measurements of torque and flow rate. The rheology modifiers and concentrations thereof were selected to create a rheology profile for precise delivery of the micronized BPO. The rheology profile for the formulation includes the yield stress and the viscosity at different shear modes and rates. The rheology profile of the formulation provides a functional formulation network, provides an easy to apply structure in viscosity, and generates favorable glide and suspension properties. The basic rheology-determining parameters of an emulsion or lotion are considered a continuous phase rheology and determine the nature of the drops (size distribution, deformability, internal viscosity, concentration of particles and particles interactions). In this Example, the selected rheology modifiers help to suspend micronized benzoyl peroxide particles evenly throughout the formulation to prevent any settling or sedimentation and help with uniform distribution and effective delivery. This prevention of settling of micronized benzoyl peroxide particles ensures consistency throughout a packaged formulation. The selected rheology modifiers control viscosity and flow to improve consistency of application of the formulation and extend useable lifetimes of a packaged formulation. Besides just viscosity, the structure and solid-like properties of micro dispersions play a crucial role in dispersion attributes and behaviors of micronized benzoyl peroxide particles. The rheology profile of the formulation provides a system that holds active ingredient BPO and helps with suspending, delivery, and uniform distribution. Xanthan gum is a rheology control agent that improves the texture, stability, and appearance of the formulation. Xanthan gum also is a naturally occurring polysaccharide that is nontoxic and nonirritating. Xanthan Gum concentration makes the formulation feel smoother and silkier upon topical application and can help reduce sensory irritation in the subject by forming a protective barrier on the skin. Carbomer primarily functions as a rheology modifier, meaning it controls the consistency and flow of the formulation by significantly increasing its viscosity, acting as a thickener to create gel form with a smooth, spreadable texture while maintaining stability and preventing separation of ingredients. This allows for even application and improved delivery of active ingredients to the skin upon topical application. By controlling viscosity, carbomer contributes to a desirable texture and feel on the skin, making the formulation easier to spread and apply. By controlling viscosity, carbomer contributes to a desirable texture and feel on the skin, making the formulation easier to spread and apply and creates an easy water breaking sensation that helps transferring active ingredient. Ammonium Acryloyldimethyltaurate/VP Copolymer forms a cross-linked polymer network that traps particles of micronized BPO, creating a stable structure and keeping the BPO particles in the form and sizes that can penetrate deeper into the acne pores. It also works as thickening and gelling agent plus as texturizer and provides a light, fresh, and non-tacky skin feel. The rheology profile of a topical formulation with micronized benzoyl peroxide generates enhanced characteristics by significantly improving an ability of the micronized benzoyl peroxide particles to spread and penetrate the skin due to their smaller micronized particle size, thereby allowing for better distribution of the BPO within the formulation. This leads to increased efficacy and reduced irritation compared to standard benzoyl peroxide particles; making the formulation smoother and easier to apply while enhancing its penetration into the skin's pores. By distributing the active ingredient of micronized benzoyl peroxide more evenly, rheology modifiers reduce irritation caused by concentrated clusters of large particles.

### Contraindications

This formulation is not to be provided to subjects that are allergic to benzoyl peroxide.

### Subject Information

In some instances, the subject is assessed to determine their skin type (e.g., normal, dry, oily, combination, sensitive, or the like), acne type and acne severity, Fitzpatrick skin complexion type, or other skin conditions and concerns to inform parameters of the skin care regimen, or any combination thereof. The formulation used and frequency of use may vary with the skin assessment of the subject, the age and physical condition of the subject, the duration of the treatment, the response and tolerability by the subject, and like factors. In some instances, the subject is a mammal. In some instances, the subject is a human. In some instances, the subject is a male. In some instances, the subject is a female. In some instances, the human subject is at least about 18 years of age. In some instances, the human subject is a child or adult 12 years of age or over. In some instances, the human subject is an adult between the ages of 18-79.

Recommendations during use of formulation to improve or clear acne: subjects are encouraged to stay sufficiently hydrated during ongoing skin care treatment regimens using this formulation.

### Usage and Tips:

The subject should apply a dime to nickel size amount to entire area, avoid neck and eyes. Let dry. The topical formulation should be applied all over the affected area; do not spot treat. Subject is recommended to apply a hydrating gel underneath the advanced acne medication formulation for added hydration.

The components and respective concentrations in the formulation are listed in Table 3 below.

TABLE 3
Formula C-Acne Medication 10% BPO

| INCI/Common Name | % w/w |
|---|---|
| Benzoyl Peroxide | 10.0-10.5 |
| Glycerin | 2.991 |
| Phenoxyethanol | 0.900 |
| Ethylhexylglycerin | 0.100 |
| Propanediol | 1.000 |
| Sodium Hydroxide | 0.160 |
| Carbomer | 0.500 |
| Ammonium | 0.400 |
| Acryloyldimethyltaurate/VP | |
| Copolymer | |
| Xanthan Gum | 0.300 |
| Sodium Hyaluronate | 0.01-0.1 |
| Bisabolol | 0.010-0.10 |
| Aqua | QS 100 |

### Example 4: Acne Hydrating Treatment Protocol for Study

Outlined below is the Hydrating Enzyme Treatment for a subject that is has dry and/or irritated skin or is pregnant or nursing. Upon assessment by a clinician, the subject may be directed to undergo an acne hydrating treatment in lieu of a facial acne peel. If the clinician determines severity of 3 or greater on a scale of 0-10 of tingling, stinging, and/or burning in a skin tolerance test with use of the glycolic-lactic toner, acne hydrating treatment is indicated as part of the customizable skin care regimen.

### Treatment Items:

Surfactant-based gel cleanser
Soothing facial toner
Papain-containing hydrating mask
Glycerin-based hydrating gel
Non-comedogenic sunscreen lotion-SPF 30 or SPF 50
Headband
Cotton round pads
Fan brush
Small bowl for enzyme
Gloves
Towel for enzyme removal
Extractor Tool

Step 1: Cleanse. The entire area to be treated is cleansed with a nickel-size amount of a surfactant-based gel cleaner and rinsed thoroughly with warm water.

Step 2: Tone. A cotton round pad is moistened with a soothing facial toner and swiped over the face evenly.

Step 3: Enzyme Treatment (can be used with or without steam). 3-4 pumps of enzyme are put in a small bowl. A thin layer of an enzyme-based chemical peel is applied with a fan brush to the area to be treated. The solution is left on for 7 minutes (5 for inflamed acne) as tolerated and removed completely with a steam towel.

Step 4: Hydrate. A generous amount of glycerin-based hydrating gel is massaged into the skin to prepare for extractions.

Step 5: Extractions: The pores are now softened from the enzyme. Extractions are performed, then the area is wiped thoroughly with a salicylic-acid and vitamin C-based toner to finish up. If the subject is pregnant or nursing, a soothing facial toner is applied instead. High frequency may be performed for additional benefits.

Finish: If the appointment is during the day, a non-comedogenic sunscreen lotion of SPF 30 is gently applied. Optionally, if the appointment is during the day, a non-comedogenic sunscreen lotion of SPF 50 is gently applied.

### Example 5: Acne Peel for Face Treatment Protocol

Outlined below is the procedure to use with acid-based chemical peel 1, acid-based chemical peel 2, and acid-based chemical peel 3. These peels contain TCA/trichloroacetic acid (5%, 7.5%, or 10%) and are not appropriate for use on pregnant or nursing subjects. If the subject is pregnant or nursing, please refer to the 20% Lactic Peel and enzyme-based chemical peel protocols.

### Treatment Items:

Surfactant-based gel cleanser
Exfoliating toner
Trichloroacetic acid-based peel solution
Salicylic-acid and Vitamin C-based toner (or Soothing facial toner if pregnant or nursing)
Sulfur-based OTC acne treatment medication
Non-comedogenic sunscreen lotion-SPF 30 or SPF 50
Peptide-containing moisturizer
Headband
Cotton round pads
4×4 esthetic wipe
Fan
Extractor Tool
Gloves

Step 1: Cleanse. The entire area to be treated is cleansed with a nickel-size amount of surfactant-based gel cleanser and rinsed thoroughly with warm water.

Step 2: Tone. Exfoliating Toner: This toner is always used before a peel treatment to degrease the skin while acting as the Skin Tolerance Test. Moisten a cotton round pad and swipe over the entire area to be treated thoroughly and evenly. The subject is asked if they are experiencing any tingling. Utilize the number scale (1-10) to help determine what the subject is feeling. On a scale of 1 to 10, 10 being extremely irritating, ask what number they are experiencing. If the subject feels a “3” or more do not proceed with a chemical peel and opt for the Acne Hydrating Treatment instead.

Step 3: Acid-based facial chemical peel. Take a 4×4 cotton esthetic wipe and fold it in half. Fold it in half one more time. Place the wipe directly onto the open bottle of acid-based chemical peel 1 solution and invert the bottle, making a circle of solution on the pad. Move the bottle around the pad and repeat this four times in a circle.

Starting on the forehead, wipe evenly and apply the peel in panels to cover the entire facial area and upper neck. Do not go over an area more than once. Ask the subject if they feel any tingling using the number scale (1-10). Fan the face to reduce the tingling. If the tingling does not calm down within two minutes, apply the peptide-containing moisturizer and continue to fan the face. This should help reduce the tingling sensation.

If the subject felt less than a “3”, apply one more layer of the acid-based chemical peel 1 solution and fan it down. If the tingling does not calm down within two minutes, apply the peptide-containing moisturizer and continue to fan their face. This should help reduce the tingling sensation. If the subject is not sensitive, use acid-based chemical peel 2 solution for the second layer. In subsequent treatments, you may opt to perform two layers of acid-based chemical peel 2, and eventually acid-based chemical peel 3. Do not apply peptide-containing moisturizer if there is not a need to reduce any tingling sensation.

Step 4: Extractions. The pores are now softened from the enzyme. Perform extractions, then wipe the area thoroughly with salicylic-acid and vitamin C-based toner to finish up. If the subject is pregnant or nursing, apply soothing facial toner instead. You may perform high frequency for additional benefits.

Step 5: Sulfur-based OTC acne treatment medication (optional). For Inflamed acne, sulfur-based OTC acne treatment medication can be applied to the affected area for calming and antibacterial action. After 3 to 5 minutes, remove the sulfur-based OTC acne treatment medication by wiping with a 4×4 esthetic wipe soaked with soothing facial toner. Do not apply if the subject is allergic to sulfur.

Step 6: Finish. If the appointment is during the day, gently apply non-comedogenic sunscreen lotion-SPF 30. If the appointment is in the evening, gently apply peptide-containing moisturizer if needed to reduce any residual tingling. Optionally, non-comedogenic sunscreen lotion
SPF 50 may be applied during the day.

### Example 6: Serum Skin Sensitivity Test

A skin sensitivity test is performed at the first initial consultation and any time the clinician plans to change or increase to a more potent serum. The serum recommendations are based on the results of the skin tolerance test. The subject's skin must be tested to determine which serum their skin can tolerate.

Knowing the subject's tolerance will guide the clinician in making the most compatible choices for their skin. Make sure the subject's home care is not too strong. Excessive at-home skin care is not better. If the home care routine is too strong, the products will eventually dehydrate and/or burn the skin and will impede the clearing process.

### Steps for Doing the Test:

1) Cleanse with surfactant-based gel cleanser and tone with soothing toner.
2) Apply 8% solution of alpha hydroxy acid-based serum to the lower side of the left cheek for inflamed, combination and moderate/severe noninflamed. Test retinaldehyde serum for mild noninflamed.
3) Ask if the subject feels any tingling, and if they do, ask, “On a scale of one to ten, one being you hardly feel anything and ten being very irritating, what number would you give it?”
4) If the subject feels a 2 or less, proceed to Step 7 below. If they feel more than a 2, then test the 5% solution of alpha hydroxy acid-based serum for inflamed or combination acne on the opposite lower cheek. If a number higher than 3 is determined, cleanse the serum off with water.
5) If the clinician proceeds to the other cheek to test 5% solution of alpha hydroxy acid-based serum, the subject is asked if they feel any tingling and if so, what degree of tingling they feel. If they feel a 2 or less, then that is the serum they will use. If a number higher than 3 is determined, cleanse the serum off with water.
6) If the subject feels more than a 2 with both 8% solution of alpha hydroxy acid-based serum and 5% solution of alpha hydroxy acid-based serum, see step 8.
7) Once the clinician finds a serum that the subject feels at a 2 or less, the serum is applied all over their face and the subject is asked if they still feel the same degree of tingling just to be sure that the subject can tolerate it. If they feel a number higher than 3, cleanse the serum off with water and (see step 8) and proceed with a treatment if planned. The exfoliating toner need not be applied prior to a peel.
8) If a subject cannot tolerate any serum (feels over a 2 on a 1-10 scale of tingling, stinging, and/or burning), do not proceed to give them one. Have them follow the home care instructions for Week 1 & 2, minus the serum. Most of the time, an advanced acne medication formulation (a micronized benzoyl peroxide formulation described one of Examples 1-3) makes their skin less sensitive over time and the test of their skin for a serum is made again in 2 weeks. IMPORTANT—If the subject feels a 0 with 8% solution of alpha hydroxy acid-based serum, there is no need to test anything higher on the face. Even if the subject can tolerate the higher strengths, the result is too much drying in the beginning of the customizable skin care regimen.

### Example 7: Efficacy and Tolerability of a Multi-Modality Treatment Method for Facial Acne Vulgaris

In this Example, an acne treatment method tailored to an individual's acne type has been developed and incorporates esthetician-administered facial treatments, at-home skincare regimens, and guidance on lifestyle and nutrition that may contribute to breakouts.

Study Objective: Evaluate the efficacy, tolerability, and safety of an acne system to improve mild to severe facial acne.

### Study Population

Approximately 60 participants are enrolled in the study. This is an open-label study; 60 participants have been deemed a large enough sample size for efficacy and safety. No randomization was conducted. Only participants with facial acne who meet the criteria for inclusion and none of the exclusion criteria are eligible for enrollment.

### Inclusion Criteria

1. Women and men over the age of 18 with no known medical conditions that, in the investigator/clinician's opinion, may interfere with study participation.
2. Participants must provide written consent and consent for photographic release.
3. A facial acne grade of mild to severe (2-4) on the Investigator's Global Assessment of Acne Severity (IGA).
4. Participants are willing to comply with the study procedures and visit schedule and use no new skincare products other than the study products provided in the treatment area for the duration of the study.
5. Willingness to avoid extended periods of sun exposure and use of tanning beds for the duration of the study.
6. Participants must be willing to refrain from any procedures to the treatment area during the study duration.

### Exclusion Criteria

1. Any dermatological disorder or poor health, which, in the investigator/clinician's opinion, may interfere with accurately evaluating the skin characteristics.
2. Participants who have demonstrated a previous hypersensitivity reaction to any of the ingredients of the study products or have any known allergies to topical products or their ingredients.
3. Participants with clinically significant unstable medical disorders.
4. Participants who are, in the opinion of the investigator/clinician, unwilling or unable to comply with the requirements of the study visits or procedures in the protocol.
5. Participants that have used retinol, steroids, or other topicals with actives on the face in the last 2 weeks, which, in the opinion of the investigator/clinician, would interfere with the study objectives.
6. Participants with recent excessive exposure to sunlight or artificial UV light (e.g., use of tanning beds/booths or sunbathing) or have used self-tanner on the face within 7 days of study entry.
7. Any facial resurfacing procedure (ablative or non-ablative laser or medium-depth chemical peel in the past month).
8. Facial waxing within 7 days of baseline or dermaplaning, dermabrasion, or any other facial treatment within 14 days that may compromise the skin barrier.
9. Use of oral isotretinoin, spironolactone, or any other oral acne medications in the last 6 months.
10. Unwilling to reduce certain dietary substances that may be associated with acne flare-ups, such as;
   a. Dairy
   b. Whey or Soy—protein shakes and bars
   c. Excessive sugar/processed foods
   d. Peanuts and other legumes
11. Currently taking high-level doses of B12 or Biotin.
12. Current user of tobacco or marijuana.
13. Participants of childbearing potential on hormonal contraceptives that have not been on a consistent dose for two months before baseline.
14. Pregnant, planning on becoming pregnant, or nursing during study participation.
15. Have been in another clinical study in the last 30 days.

### Study Methods

Participants undergo up to 9 study visits, including one screening and baseline visit, which may be combined if the participant meets eligibility to be enrolled on the same day. At the initial screening visit, which may be completed virtually, participants are consented prior to any study procedures. Medical history, concomitant medications, topical products, and procedures are collected. Study eligibility is assessed, and if a participant requires a washout of exclusionary products or procedures/treatments, they return to the office after the appropriate time frame for the baseline visit. At the baseline visit, the following is completed: Clinician assessment, participant assessment, baseline photography, and skin sensitivity test or a skin tolerance test. Participants have an in-office acne treatment by a trained clinician according to their acne skin type. Participants are given a skincare regimen according to their acne skin type to use twice daily at-home. Participants are asked to return to the office for follow-up visits every 2 weeks until the study ends at week 16. At each follow-up visit, the same procedures are completed.

### Photography

Photographs are taken at all visits using a camera system with consistent placement and conditions. Photographs are taken prior to any product application (clean and dry skin only). Any photo used for publication or marketing purposes has consent for use. Participants must agree to have their photographs taken to participate in this study.

### Study Products

Participants receive an acne skin care regimen according to their skin characteristics and type of acne. All participants undergo a skin tolerability/sensitivity test prior to dispensing the study products. Participants receive adequate amounts of topical products for at-home application twice daily.

### Study Visits

An initial screening visit was conducted to assess study enrollment criteria for potential study participants. In this initial screening visit, he clinician and/or study personnel discussed the study with potential participants. Participants consented and assessed for eligibility to enroll in the study. If a participant required discontinuation of exclusionary products or procedures/treatments, they returned to the office after the appropriate time frame for the baseline visit.

A baseline visit was conducted for reassess eligibility to enroll in the study and any additions or changes to the subject's medical history and (concomitant medications, topical products, and procedures/treatments). The baseline visit may be combined with the initial screening visit.
During the baseline visit, this following was undertaken;
Investigator/Clinician assessment of facial skin and acne assessment (IGA).
Participant assessment and questionnaire.
Photography of clean, dry facial skin (5 views post 5-10 minutes of cleansing).
Description of Skin Serum Sensitivity completed by Clinician:
   1. Cleanse with surfactant-based gel cleaner and toner with soothing toner.
   2. Apply 8% solution of alpha hydroxy acid-based serum to the lower side of the left cheek for inflamed, combination, and moderate/severe noninflamed. Test retinaldehyde serum for mild noninflamed.
   3. Ask if they the participant feels any tingling and if they do, ask, “On a scale of one to ten, one being you hardly feel anything and ten being very irritating, what number would you give it?”
   4. If the participant feels a 2 or less, proceed to Step 7 below. If they feel more than a 2, the 5% solution of alpha hydroxy acid-based serum is tested for inflamed or combination acne on the opposite lower cheek. Cleanse the serum off with water if you get a number higher than 3.
   5. If you are proceeding to the other cheek to test 5% solution of alpha hydroxy acid-based serum, ask if the participant feels any tingling and, if so, what degree of tingling they feel. If they feel a 2 or less, then that is the serum used. Cleanse the serum off with water if you get a number higher than 3.
   6. If the participant feels more than a 2 with both 8% solution of alpha hydroxy acid-based serum and 5% solution of alpha hydroxy acid-based serum, see step 8.
   7. Once you find a serum that the participant feels at a 2 or less, put the serum all over their face and ask if they still feel the same degree of tingling to ensure tolerability. If they feel a number higher than 3, cleanse the serum off with water (see step 8) and proceed with a treatment if planned. You do not need to apply the exfoliating toner prior to a peel.
   8. If a participant cannot tolerate any serum (feels over a 2 on a 1-10 scale of tingling, stinging, or burning), do not dispense one. Have them follow the home care instructions for Weeks 1 & 2, excluding the serum. Test their skin for a serum again in 2 weeks.
   9. IMPORTANT—If they feel a 0 with 8% solution of alpha hydroxy acid-based serum, there is no need to test anything higher on the face. Even if they can tolerate the higher strengths, they are too drying initially.
Clinician Facial Procedure.
Dispense the acne skincare regimen with instructions for use.

The following information was collected for the participant questionnaire during the initial screening visit. The participant questionnaire may be repeated during baseline visit and during any or all of the follow-up visits. The prospective participant or enrolled participant completed the following:
Please indicate if you have used any of the medications or drugs listed below in the last 2 years, when they were used, and for how long you used them.

| | | HOW |
|---|---|---|
| MEDICATION | WHEN | LONG |
| Antibiotics (oral) | | |
| Antibiotics (topical) | | |
| Isotretinoin | | |
| Benzoyl Peroxide | | |
| Tretinoin, tazarotene, or | | |
| adapalene gel 0.1% | | |
| Thyroid medication | | |
| Blood Thinning Meds | | |

Please list any other medications or drugs listed that you have used in the past 2 years and include when they were used, and for how long you used them.
   Medical History (Please Check all that Apply)

| | | |
|---|---|---|
| Herpes Simplex | HIV/AIDS | Hemophilia |
| Eczema | Thyroid Problems | Lupus |
| Psoriasis | Hormone Problems | Anemia |
| Hepatitis | Hysterectomy | High Blood Pressure |
| Cancer | Ovary(ies) Removed | Diabetes |
| Staph | Pacemaker | Metal Pins in Body |
| Infection/MRSA | | |

Are you under a dermatologist's or other physician's care? If yes, provide doctor's name.
Lifestyle considerations:
   Have you ever had any reaction to any products or anything you have put on your face? If yes, what products?
   Please check any of these you are allergic to: 1) Sulfur 2) Aspirin 3) Latex
   List any other allergies you know of:
   Do you smoke/vape? If yes, what do you smoke?
   Do you use fabric
   softener or fabric softener sheets in the dryer?
   Do you swim in a chlorinated pool?
   Do you work around chemicals, tars, oils, grease, or inks?
   List occupation. Do you work nights?
   Are you currently under a lot of stress? (common stress triggers include: job loss, new job, wedding, death in the family or close friend, graduation, long commute, heavily scheduled)
   Do you use birth control pills, shots or use an TUD? If so, which do you use and what brand product?
   Are you pregnant or nursing?
   Do you have shaving irritation on your face?
   What type of razor do you use for shaving (e.g., double blade, triple blade, rotary blade)
Diet—List any of the following that are consumed as part of your diet and frequency of consumption

| | HOW OFTEN | | HOW OFTEN |
|---|---|---|---|
| FOODS | PER WEEK | FOODS | PER WEEK |
| Fast Food | | Peanuts | |
| Processed Food | | Sushi | |
| Salty Snacks | | Kelp and Seaweed | |
| Milk/Yogurt | | Miso Soup | |
| Cheese | | Soy | |
| Whey or Soy | | Vitamins/ | |
| Protein | | Supplements | |
| Peanut Butter | | Seafood | |

List products currently using and provide product names below:

| Product Category | Product Name |
|---|---|
| Cleanser | |
| Toner | |
| Serums | |
| Moisturizers | |
| Sunscreen | |
| Mask | |
| Foundation | |
| Blush | |
| Exfoliants (acids, serums, | |
| scrubs) | |
| Acne medications | |
| Any other products | |

List other skin procedures undertaken within the last 90 days as indicated below. Please indicate date of procedures and location

| Treatment | When | Where |
|---|---|---|
| Chemical Peels | | |
| If so, what kind: | | |
| Microdermabrasion | | |
| Dermabrasion | | |
| Laser Hair Removal | | |
| Laser Rejuvenation/Resurfacing | | |
| Skin Cancer Removal | | |
| Facial Waxing | | |
| Electrolysis | | |
| Other: | | |

### Follow-Up Visits

Follow-up visits were conducted from between Weeks 2-16 of the study. Participants returned for follow-up visits at the clinic every 2 weeks (+/−3 days). If a participant missed a visit and was not able to return until the next scheduled visit, a virtual follow-up was performed if possible.

The following procedures were performed at follow-up visits:
Assess and record any additions or changes in concomitant medications and topical products or procedures to the treatment area.
Record any Adverse Events and assess the tolerability of the products (erythema or redness, peeling, dryness, stinging, and/or burning).
Photography of clean, dry facial skin (5 views post 10 minutes of cleansing).
Investigator/Clinician assessment of facial skin and acne assessment (IGA), skin tolerability, and appearance.
Participant assessment and questionnaire.
Skin tolerance test to be completed (if required).
Clinician Facial Procedure.
Dispense additional products as needed for the acne skincare regimen.

Week 16: Participants returned for a final follow-up visit at Week 16 (+/−5 days). The following procedures were performed:
Assess and record any additions or changes in concomitant medications and topical products or procedures to the treatment area.
Record any Adverse Events and assess the tolerability of the products (erythema or redness, peeling, dryness, stinging, and burning).
Photography of clean, dry facial skin (5 views post 10 minutes of cleansing).
Investigator/Clinician assessment of facial skin and acne assessment (IGA).
Participant assessment and questionnaire.
Clinician Facial Procedure.
   Restrictions within Study:
Excessive exposure to sunlight or artificial UV light (e.g., use of tanning beds/booths and sunbathing).
Use of other topical products other than the study products.
Procedures or treatments on the facial area during the study.
The addition or modification of makeup, hair care, or any other topical on the area being treated for acne must be approved and recorded prior to use by the Investigator/Clinician.
Pregnant or planning on becoming pregnant during the course of study participation through self-reporting at all visits.

### Study Participant Withdrawal and Discontinuation:

After admission to the study, the participant may withdraw at any time for any reason. The reason for withdrawal will be documented. A participant may be discontinued if 2 visits are missed within a month or if they cannot follow the skin care regimen or visit schedule. A participant who becomes pregnant during the study will be discontinued.

### Assessments:

The following clinical skin care grading system and categorization of acne severity was used according to the Investigator's Global Assessment (IGA) of acne severity

Investigator's Global Assessment (IGA) of acne severity

| | | |
|---|---|---|
| 0 | ‘Clear’ | Residual hyperpigmentation and |
| | | erythema may be present |
| 1 | ‘Almost clear’ | A few scattered comedones and |
| | | a few small papules |
| 2 | ‘Mild’ | Easily recognizable; less than |
| | | half the face is involved. Some |
| | | comedones and some |
| | | papules and pustules |
| 3 | ‘Moderate’ | More than half the face is |
| | | involved. Many comedones, |
| | | papules and pustules. One |
| | | nodule may be present |
| 4 | ‘Severe’ | Entire face is involved, covered |
| | | with comedones, numerous |
| | | papules and pustules, and few |
| | | nodules and cysts |

Additional skin parameters were also assessed:

| | | | | | | | | | | |
|---|---|---|---|---|---|---|---|---|---|---|
| Parameter | 0 | 1 | 2 | 3 | 4 | 5 | 6 | 7 | 8 | 9 |

| | | |
|---|---|---|
| Pore Size | 0 = Pores | 9 = Pores are |
| | are small | large and |
| | and not | prominent |
| | noticeable | |
| Overall | 0 = No | 9 = Overall, |
| Redness of | redness | inflammatory |
| Inflammatory | associated | lesions exhibit |
| Lesions | with the | severe degree of |
| | inflammatory | redness |
| | lesions | |
| Overall Size of | 0 = No | 9 = Overall, |
| Inflammatory | longer | size is very |
| Lesions | visible | large |

Tolerability was assessed for each of the following features in facial skin: erythema (redness), peeling, dryness, and stinging/burning according to the grading system below.

| Grade | Description |
|---|---|
| 0 | Absent |
| 1 | Trace |
| 2 | Slight |
| 3 | Moderate |
| 4 | Prominent |

The following participant questionnaires were completed:

| | | | | | | |
|---|---|---|---|---|---|---|
| How would | Clear | Almost | Mild | Moderate | Severe | Very Severe |
| you | | Clear | | | | |
| describe | | | | | | |
| your acne | | | | | | |
| at the | | | | | | |
| moment? | | | | | | |
| How much | 0 = No | 1 = | 2 = | 3 = Good | 4 = Very | 5 = |
| has your | Improvement | Minimal | Moderate | Improvement | Good | Excellent |
| acne | | Improvement | Improvement | | Improvement | Improvement |
| improved | | | | | | |
| since | | | | | | |
| starting the | | | | | | |
| acne | | | | | | |
| skincare | | | | | | |
| regimen? | | | | | | |

Since using the acne skin care regimen, check the box below that describes your answer to the statement:

| | | | | | | |
|---|---|---|---|---|---|---|
| | | | Neither | | | |
| Strongly | | Somewhat | Agree nor | Somewhat | | Strongly |
| Agree | Agree | Agree | Disagree | Disagree | Disagree | Disagree |

Lessened the
appearance
of acne on
my skin
Made my
skin look
less oily
Lessened the
appearance of
discoloration
on my skin
Improved
the evenness
of my skin
tone
Made me
feel more
confident in
the way my
skin looks
Made my
skin look
healthier
Made me
look and feel
better
Improved
the overall
appearance
of my skin
Using this
regimen
gave me a
feeling of
hope
Using this
regimen
made me
feel
supported

### Week 16 Study Visit Questions:

| Last Study Visit | Very | | | | Very |
|---|---|---|---|---|---|
| Questions: | Likely | Likely | Neutral | Unlikely | Unlikely |
| I would continue | | | | | |
| using this acne | | | | | |
| skincare regimen | | | | | |
| I would | | | | | |
| recommend | | | | | |
| this acne skincare | | | | | |
| regimen to others | | | | | |

### Data Analysis:

All data points collected during the study are compared to baseline. Data collected from discontinued participants may be included in the statistical analysis. An independent statistician performs statistical analysis. At the end of the study, an independent blinded dermatologist may review the study photos to assess the efficacy of the study products on the skin.

### Risks:

Possible risks from the study products could include skin irritations, contact dermatitis, and hypersensitivities ranging from itchiness and redness to peeling. Most resolve spontaneously.

### Adverse Events:

An adverse event (AE) is defined as any adverse health-related event associated with the use of a cosmetic product. A serious adverse event (SAE) is defined as death, a life-threatening adverse experience, inpatient hospitalization, a persistent or significant disability/incapability, or a congenital anomaly/birth defect. Serious adverse events are reported to the sponsor within 24 hours of the investigative personnel's knowledge of the event. All AEs, whether observed by the clinical staff or by the participant, and whether or not thought to be related, are recorded on an Adverse Event form. The study staff asks the participant about any adverse reactions experienced since the last visit. Participants are also instructed to be contacted the study site if they experience any adverse events.

Participant Privacy Protection: All participants who enroll in the study are given a unique code that is their participant number and initials. All information pertaining to the participant is de-identified.

Monitoring of the study: The sponsor may make site visits during the study and may inspect all forms and other documentation directly associated with the study.

Study Design Alteration: No alteration is made to this protocol without written documentation and sponsor and IRB approval. Written approval is in the form of an amendment attached to the final protocol. If the Investigator/Clinician determines that an immediate alteration to the protocol is required to protect the safety of the participants, this is documented and brought to the sponsor's attention.

### Confidential Information:

All information supplied by the Sponsor in connection with this study and not previously published is considered confidential information. This information includes, but is not limited to, the clinical protocol, case report forms, and other scientific data. Any data collected during the study is considered confidential. This confidential information shall remain the sole property of the Sponsor and shall not be disclosed to others without the written consent of the Sponsor.

The information developed during the conduct of this clinical study is also considered confidential and may be used by the Sponsor in connection with the marketing of the study product and regimen. The information may be disclosed as deemed necessary by the Sponsor. To allow the use of the information derived from this clinical study, the investigator/clinician is obliged to provide the Sponsor with complete test results and all data developed in this study. Investigators/Clinicians and Sponsor will collaborate on the publishing of any manuscript. Study data will be kept in a secure location only accessible by limited study staff, and all identifying information of participants will be protected from improper use and disclosure. All data collected during the study will be retained as per applicable regulations.

The below is a schedule for procedures undertaken at various time points of the study. The screening visit and the baseline visit may be performed on the same day if a washout is not required.

Schedule for Procedures

| | | Week | Week | Week | Week | Week | Week | Week |
|---|---|---|---|---|---|---|---|---|
| Screening | Baseline | 2 | 4 | 8 | 10 | 12 | 14 | 16 |

Informed Consent
Medical/Topical History
Demographics
Inclusion/Exclusion
Criteria
Concomitant
Medications/Topical Query
Adverse Event Query
Photography
Clinician Facial Acne
Assessment
Clinic Facial Procedure
Participant
Assessment/Questionnaire
Dispense Products with
Instructions for Use

### Adaptive System:

Throughout the duration of the skin care regimen implemented in this study, continued assessment of conditions and improvement within the facial skin of each participant is undertaken. At the baseline visit, the participant undergoes a facial acne assessment by a clinician or individual trained in clinician skin grading according to IGA of acne severity. The results of this assessment are recorded and may be combined with additional participant information collected and recorded including medical/topical history, participant demographics, concomitant medications, previous adverse events, current and/or previous skin care regimen products and methods used, Fitzpatrick skin type system categorization of the participant, and any additional information collected from the participant assessment and questionnaire. This combined assessment and information is used to recommended clinical facial procedure to be undertaken at the time of the baseline visit. The recommended clinical facial procedure comprises a chemical peel that is selected according to participant's current facial skin conditions and background information. Also, during the baseline visit, the clinician or individual trained in clinician skin grading establishes an ongoing daily skincare regimen between visits comprising use of a recommended acne clearing formulation described herein. The adaptive aspects of this system continue with additional clinician assessment conducted during each follow-up visit. During each follow-up visit, the participant is assessed and undergoes a recommended clinical facial procedure comprising a chemical peel according to the participant's current facial skin condition, determination of acne severity, and accompanied information from baseline and any additional assessments from previous follow-up visits. Also, during each baseline visit, the clinician or individual trained in clinician skin grading reassesses the ongoing daily skincare regimen comprising use of the recommended acne clearing formulation described herein and makes any changes necessary according to progress or outcome of the ongoing skin care regimen conducted by the participant in between visits. In some cases, the participant is instructed to continue following the parameters of the most recent skin care regimen. In some cases, the participant is instructed to adjust the parameters of the most recent skin care regimen to using a different acne clearing formulation or associated skin cleaning products. Reassessment continues by the clinician during subsequent follow-up visits until a sufficient improvement in acne clearance in the participant is achieved. In some instances, the participant achieves at least an IGA acne severity assessment of 0 ‘clear’ or 1 ‘almost clear’. This specialized, adaptive multi-modal system for clearing facial acne considers an individual's specific skin type, acne severity, and continued response for acne clearance. Customized modifications to the participant's skin care regiment are based upon response to treatment.

Results: Sixty participants completed the study. IGA scores demonstrated a statistically significant improvement in acne at all time points, beginning at week 2 and continuing to improve through week 16, (p<0.0001), with at least a two-grade improvement from baseline achieved at Week 14 (72%, p=0.001) and at Week 16 (80%, p<0.0001). Participant skin assessment grades demonstrated statistically significant improvement at all follow-up visits (p<0.0001). All participants tolerated individual regimens well with no reported adverse events.

Conclusions: The multi-modality method for treating acne demonstrated significant improvements in facial acne over the 16-week study.

While preferred aspects of the present inventive concepts have been shown and described herein, it will be obvious to those skilled in the art that such aspects are provided by way of example only. Numerous variations, changes, and substitutions will now occur to those skilled in the art without departing from the inventive concepts. It should be understood that various alternatives to the aspects of the inventive concepts described herein may be employed in practicing the inventive concepts. It is intended that the following claims define the scope of the inventive concepts and that methods and structures within the scope of these claims and their equivalents be covered thereby.

### Aspects

Aspect 1: A method for clearing inflamed or combination acne in a subject in need thereof, the method comprising:
i) assessing an extent of acne severity in the subject;
ii) determining a skin type of the subject;
iii) assigning a customizable skin care regimen to the subject, wherein the customizable skin care regimen is initiated by the subject and comprises:
   a. cleansing the skin of the subject with facial cleanser;
   b. optionally applying ice to the skin of the subject following the cleansing;
   c. applying a toner to the skin of the subject following the cleansing in a), or the optional application of the ice in b);
   d. applying a serum to the skin of the subject following the application of the toner;
   e. applying a topical bactericidal-based acne treatment formulation to the skin of the subject following the application of the serum, wherein the topical bactericidal-based acne treatment formulation is selected from:
      i. a benzoyl peroxide-based acne treatment topical formulation;
      ii. a salicylic acid-based acne treatment topical formulation; or
      iii. a sulfur-based acne treatment topical formulation; and
iv) conducting an in-clinic assessment and treatment comprising at least one facial acne peel or an acne hydrating treatment, wherein the assessment and treatment are each administered by a clinician.

Aspect 2: A method for clearing inflamed or combination acne in a subject in need thereof, the method comprising:
i) assessing an extent of acne severity in the subject;
ii) determining a skin type of the subject;
iii) assigning a customizable skin care regimen to the subject, wherein the customizable skin care regimen is initiated by the subject and comprises:
   a. cleansing the skin of the subject with facial cleanser,
   b. optionally applying ice to the skin of the subject following the cleansing;
   c. applying a toner to the skin of the subject following the cleansing in a), or the optional application of the ice in b);
   d. applying a serum to the skin of the subject following the application of the toner;
   e. applying a topical bactericidal-based acne treatment formulation to the skin of the subject following the application of the serum, wherein the topical bactericidal-based acne treatment formulation is selected from:
      i. a benzoyl peroxide-based acne treatment topical formulation;
      ii. a salicylic acid-based acne treatment topical formulation; or
      iii. a sulfur-based acne treatment topical formulation;
   f. applying a hydrator or a moisturizer to the skin of the subject;
   g. applying a sunscreen to the skin of the subject daily in the AM, and
iv) conducting an in-clinic assessment and treatment comprising at least one facial acne peel or an acne hydrating treatment, wherein the assessment and treatment are each administered by a clinician.

Aspect 3: The method of aspect 2, wherein the customizable skin care regimen comprises applying a hydrator and a moisturizer to the skin of the subject.

Aspect 4: The method of aspect 2 or 3, wherein the sunscreen is a broad-spectrum sunscreen.

Aspect 5: The method of aspect 4, wherein the broad-spectrum sunscreen is a mineral-based sunscreen.

Aspect 6: The method of aspect 5, wherein the mineral-based sunscreen is rated to at least SPF 50.

Aspect 7: The method of aspect 4, wherein the broad-spectrum sunscreen is a chemical-based sunscreen.

Aspect 8: The method of any one of aspects 4-7, wherein the broad-spectrum sunscreen is a non-comedogenic sunscreen.

Aspect 9: The method of aspect 8, wherein the non-comedogenic sunscreen is rated to at least SPF 30 or to at least SPF 50.

Aspect 10: The method of any one of aspects 1-9, wherein the in-clinic assessment and treatment begin on or around the same starting date as the customizable skin care regimen begins, and wherein the in-clinic assessment and treatment are recurrently conducted by the clinician until the subject has achieved a significant extent of clearance of inflamed acne or combination acne.

Aspect 11: The method of aspect 10, wherein the in-clinic assessment and treatment are used to revise the customizable skin care regimen to achieve favorable acne clearance and improved facial skin condition in the subject.

Aspect 12: The method of aspect 10 or 11, wherein the in-clinic assessment and treatment are used to revise the customizable skin care regimen to improve tolerance to skin care products selected as part of the customizable skin care regimen.

Aspect 13: The method of any of the preceding aspects, wherein the subject has mild, moderate, or severe inflamed or combination acne at the time of a first in-clinic assessment.

Aspect 14: The method of aspect 13, wherein the subject is assigned an initial skin care routine for twice daily use at home.

Aspect 15: The method of aspect 14, wherein the initial skin care routine is customized to the skin type of the subject.

Aspect 16: The method of aspect 15, wherein the skin type of the subject is (i) dry, (ii) mature, (iii) Fitzpatrick type 4, 5, or 6, (iv) normal, (v) oily and Fitzpatrick type 4, 5, or 6, (vi) oily, or (vii) oily in a subject that is pregnant or nursing (oily P/N).

Aspect 17: The method of aspect 16, wherein the skin type of the subject is (i) dry, (ii) mature, or (iii) Fitzpatrick type 4, 5, or 6, and wherein the cleanser is an amino acid surfactant-based cleanser.

Aspect 18: The method of aspect 16 or 17, wherein the skin type of the subject is (i) dry, and wherein the toner is a hydrating toner.

Aspect 19: The method of any one of aspects 16-18, wherein the skin type of the subject is (i) dry, (ii) mature, or (iii) Fitzpatrick type 4, 5, or 6, and wherein the serum is a 5% solution of an alpha hydroxy acid-based serum or an 8% solution of an alpha hydroxy acid-based serum.

Aspect 20: The method of any one of aspects 16-19, wherein the skin type of the subject is (i) dry, (ii) mature, or (iii) Fitzpatrick type 4, 5, or 6, and wherein the topical bactericidal-based acne treatment formulation comprises about 2.5% micronized benzoyl peroxide.

Aspect 21: The method of any one of aspects 16-20, wherein a peptide-containing moisturizer is optionally applied to the face of the subject.

Aspect 22: The method of aspect 16, wherein the skin type of the subject is (iv) normal, and wherein the cleanser is a surfactant-based gel cleanser.

Aspect 23: The method of aspect 22, wherein the skin type of the subject is (iv) normal, and wherein the toner is a soothing facial toner.

Aspect 24: The method of aspect 22 or 23, wherein the skin type of the subject is (iv) normal, and wherein the serum is a 5% solution of an alpha hydroxy acid-based serum or an 8% solution of an alpha hydroxy acid-based serum.

Aspect 25: The method of any one of aspects 22-24, wherein the skin type of the subject is (iv) normal, and wherein the topical bactericidal-based acne treatment formulation comprises about 5% micronized benzoyl peroxide.

Aspect 26: The method of aspect 16, wherein the skin type of the subject is (v) oily and Fitzpatrick type 4, 5, or 6, and wherein the cleanser is a surfactant-based gel cleanser.

Aspect 27: The method of aspect 26, wherein the skin type of the subject is (v) oily and Fitzpatrick type 4, 5, or 6, and wherein the toner is a soothing facial toner.

Aspect 28: The method of aspect 26 or 27, wherein the skin type of the subject is (v) oily and Fitzpatrick type 4, 5, or 6, wherein the serum is a 5% solution of an alpha hydroxy acid-based serum or an 8% solution of an alpha hydroxy acid-based serum.

Aspect 29: The method of any one of aspects 26-28, wherein the skin type of the subject is (v) oily and Fitzpatrick type 4, 5, or 6, wherein the topical bactericidal-based acne treatment formulation comprises about 2.5% micronized benzoyl peroxide.

Aspect 30: The method of aspect 16, wherein the skin type of the subject is (vi) oily, and wherein the toner is a salicylic-acid and vitamin C-based toner.

Aspect 31: The method of aspect 16, wherein the skin type of the subject is (vii) oily P/N, and wherein the toner is a soothing facial toner.

Aspect 32: The method of aspect 30 or 31, wherein the skin type of the subject is (vi) oily or (vii) oily P/N, and wherein the cleanser is a surfactant-based gel cleanser or a surfactant-based exfoliating gel cleanser.

Aspect 33: The method of aspect 32, wherein the skin type of the subject is (vi) oily or (vii) oily P/N, and wherein the serum is a 5% solution of an alpha hydroxy acid-based serum or an 8% solution of an alpha hydroxy acid-based serum.

Aspect 34: The method of aspect 32 or 33, wherein the skin type of the subject is (vi) oily or (vii) oily P/N, and wherein the topical bactericidal-based acne treatment formulation comprises about 5% micronized benzoyl peroxide.

Aspect 35: The method of any the preceding aspects, wherein the applying ice to the skin of the subject is done for between about 5-90 seconds.

Aspect 36: The method of any the preceding aspects, wherein the optionally applying ice to the skin of the subject is done for at least about 5, 10, 15, 20, 25, 30, 35, 40, 45, 50, 55, 60, 70, 80, or 90 seconds.

Aspect 37: The method of any the preceding aspects, wherein the optionally applying ice to the skin of the subject is done for less than about 90, 60, 45 or 30 seconds.

Aspect 38: The method of any the preceding aspects, wherein an optional hydrator is applied to the skin of the subject and wherein the optional hydrator maintains skin of the subject in a healthy state of hydration so that excess oil is not produced.

Aspect 39: The method of aspect 38, wherein the optional hydrator is a glycerin-based hydrating gel.

Aspect 40: The method of any one of aspects 22-39, wherein an ester-based moisturizer is optionally applied to the skin of the subject depending on an extent of facial skin dryness.

Aspect 41: The method of any the preceding aspects, wherein starting from Week 3 onward in the customizable skin care regimen, sulfur-based over the counter (OTC) acne medication is applied to areas of the facial skin of the subject having inflamed acne breakouts.

Aspect 42: The method of any the preceding aspects, wherein starting from Week 7 onward in the customizable skin care regimen, the strength of the micronized benzoyl peroxide in the topical bactericidal-based acne treatment formulation used by the subject in the customizable skin care regimen is increased if the subject has not achieved sufficient acne clearance.

Aspect 43: The method of any the preceding aspects, wherein starting from Week 7 onward in the customizable skin care regimen, the strength of the micronized benzoyl peroxide in the topical bactericidal-based acne treatment formulation is increased if total inflamed acne count is the same or more than during a previous in-clinic assessment and treatment session and if the total noninflamed acne count is less, the same, or more than during the most recent in-clinic assessment and treatment session.

Aspect 44: The method of any the preceding aspects, wherein starting from Week 7 onward in the customizable skin care regimen, the strength of the serum is increased one level if total inflamed acne count is the same or more than during a previous in-clinic assessment and treatment session and the total noninflamed acne count is the same or more than during the most recent in-clinic assessment and treatment session.

Aspect 45: The method of any one of aspects 1-41, wherein the serum is changed to a retinaldehyde serum if the total inflamed acne count is zero and if the total noninflamed acne count is the same or more than during the most recent in-clinic assessment and treatment session.

Aspect 46: The method of any the preceding aspects, wherein no changes are made to an ongoing customizable skin care regimen if the total inflamed acne count and the total noninflamed acne count are both reduced.

Aspect 47: The method of any the preceding aspects, wherein a facial acne peel is administered by a clinician to the subject at the start of treatment in Week 1, in Week 3, in Week 5, in Week 7, in Week 9, in Week 11, in Week 13, or in Week 15, or any combination thereof if tolerated by the subject.

Aspect 48: The method of any the preceding aspects, wherein an acne hydrating treatment is administered by a clinician to the subject at the start of treatment, at Week 1, at Week 3, at Week 5, at Week 7, at Week 9, at Week 11, at Week 13, or at Week 15, or any combination thereof upon determining that an acne peel will not be well-tolerated by the subject at the time.

Aspect 49: A method for clearing noninflamed acne in a subject in need thereof, the method comprising:
i) assessing an extent of acne severity in the subject;
ii) determining a skin type of the subject;
iii) assigning a customizable skin care regimen to the subject, wherein the customizable skin care regimen is initiated by the subject and comprises:
   a. cleansing the skin of the subject with facial cleanser;
   b. applying a toner to the skin of the subject following the cleansing;
   c. applying a serum to the skin of the subject following the application of the toner;
   d. applying a topical bactericidal-based acne treatment formulation to the skin of the subject following the application of the serum, wherein the topical bactericidal-based acne treatment formulation is selected from:
      i. a benzoyl peroxide-based acne treatment topical formulation;
      ii. a salicylic acid-based acne treatment topical formulation; or
      iii. a sulfur-based acne treatment topical formulation; and
iv) conducting an in-clinic assessment and treatment comprising at least one facial acne peel or an acne hydrating treatment, wherein the assessment and treatment are each administered by a clinician.

Aspect 50: A method for clearing noninflamed acne in a subject in need thereof, the method comprising:
i) assessing an extent of acne severity in the subject;
ii) determining a skin type of the subject;
iii) assigning a customizable skin care regimen to the subject, wherein the customizable skin care regimen is initiated by the subject and comprises:
   a. cleansing the skin of the subject with facial cleanser;
   b. applying a toner to the skin of the subject following the cleansing;
   c. applying a serum to the skin of the subject following the application of the toner;
   d. applying a topical bactericidal-based acne treatment formulation to the skin of the subject following the application of the serum, wherein the topical bactericidal-based acne treatment formulation is selected from:
      i. a benzoyl peroxide-based acne treatment topical formulation;
      ii. a salicylic acid-based acne treatment topical formulation; or
      iii. a sulfur-based acne treatment topical formulation;
   e. applying a moisturizer to the skin of the subject;
   f. applying a sunscreen to the skin of the subject daily in the AM; and
iv) conducting an in-clinic assessment and treatment comprising at least one facial acne peel or an acne hydrating treatment, wherein the assessment and treatment are each administered by a clinician.

Aspect 51: The method of aspect 49 or 50, wherein the in-clinic assessment and treatment begin on or around the same starting date as the customizable skin care regimen begins, and wherein the in-clinic assessment and treatment are recurrently conducted by the clinician until the subject has achieved a significant extent of clearance of inflamed acne or combination acne.

Aspect 52: The method of aspect 51, wherein the in-clinic assessment and treatment are used to revise the customizable skin care regimen to achieve favorable acne clearance and improved facial skin condition in the subject.

Aspect 53: The method of aspect 51 or 52, wherein the in-clinic assessment and treatment are used to revise the customizable skin care regimen to improve tolerance to skin care products selected as part of the customizable skin care regimen.

Aspect 54: The method of any one of aspects 49-53, wherein the subject has moderate or severe noninflamed acne, and wherein the subject does not have inflamed acne.

Aspect 55: The method of aspect any one of aspects 49-54, wherein the subject is assigned an initial skin care routine for twice daily use at home.

Aspect 56: The method of aspect 55, wherein the initial skin care routine is customized to the skin type of the subject.

Aspect 57: The method of aspect 56, wherein the skin type of the subject is (i) dry, (ii) mature, (iii) Fitzpatrick type 4, 5, or 6, (iv) normal, (v) oily and Fitzpatrick type 4, 5, or 6, (vi) oily, or (vii) oily in a subject that is pregnant or nursing (oily P/N).

Aspect 58: The method of aspect 57, wherein the skin type of the subject is (i) dry, (ii) mature, or (iii) Fitzpatrick type 4, 5, or 6, and wherein the cleanser is an amino acid surfactant-based cleanser or a sulfate-free gel cleanser.

Aspect 59: The method of aspect 57, wherein the skin type of the subject is (i) dry, (ii) mature, or (iii) Fitzpatrick type 4, 5, or 6, and wherein the toner is a humectant-based toner.

Aspect 60: The method of aspect 58 or 59, wherein the skin type of the subject is (i) dry, (ii) mature, or (iii) Fitzpatrick type 4, 5, or 6, and wherein the serum is a 5% solution of an alpha hydroxy acid-based serum or an 8% solution of an alpha hydroxy acid-based serum.

Aspect 61: The method of any one of aspects 58-60, wherein the skin type of the subject is (i) dry, (ii) mature, or (iii) Fitzpatrick type 4, 5, or 6, and wherein the topical bactericidal-based acne treatment formulation comprises about 2.5% micronized benzoyl peroxide.

Aspect 62: The method of any one of aspects 58-61, wherein an optional peptide-containing moisturizer is applied to the face of the subject.

Aspect 63: The method of aspect 57, wherein the skin type of the subject is (iv) normal or (v) oily and Fitzpatrick type 4, 5, or 6, and wherein the cleanser is a surfactant-based gel cleanser.

Aspect 64: The method of aspect 57, wherein the skin type of the subject is (vi) oily, or (vii) oily P/N, and wherein the cleanser is a surfactant-based gel cleanser or a surfactant-based exfoliating gel cleanser.

Aspect 65: The method of aspect 63 or 64, wherein the toner is a soothing facial toner.

Aspect 66: The method of any one of aspects 63-65, wherein the serum is a 5% solution of an alpha hydroxy acid-based serum or an 8% solution of an alpha hydroxy acid-based serum.

Aspect 67: The method of any one of aspects 63, 65, or 66, wherein the skin type of the subject is (v) oily and Fitzpatrick type 4, 5, or 6, and wherein the topical bactericidal-based acne treatment formulation comprises about 2.5% micronized benzoyl peroxide.

Aspect 68: The method of any one of aspects 63-67, wherein the skin type of the subject is (iv) normal, (vi) oily, or (vii) oily P/N, and wherein the topical bactericidal-based acne treatment formulation comprises about 5% micronized benzoyl peroxide.

Aspect 69: The method of any one of aspects 63-68, wherein the skin type of the subject is (iv) normal, (v) oily and Fitzpatrick type 4, 5, or 6, (vi) oily, or (vii) oily P/N, and wherein the serum is a 5% solution of an alpha hydroxy acid-based serum or an 8% solution of an alpha hydroxy acid-based serum.

Aspect 70: The method of any one of aspects 49-69, wherein an optional hydrator is applied to the skin of the subject and wherein the optional hydrator maintains skin of the subject in a healthy state of hydration so that excess oil is not produced.

Aspect 71: The method of aspect 70, wherein the optional hydrator is a glycerin-based hydrating gel.

Aspect 72: The method of any one of aspects 50-63, wherein an ester-based moisturizer is optionally applied to the skin of the subject depending on an extent of facial skin dryness.

Aspect 73: The method of any one of aspects 49-72, wherein from Week 7 onward if the total noninflamed acne count increases compared to a previous assessment, application of a retinaldehyde serum is added to the customizable skin care regimen in each evening.

Aspect 74: The method of any one of aspects 49-73, wherein from Week 7 onward if the total noninflamed acne count is reduced from the most recent assessment, no changes are made in the customizable skin care regimen.

Aspect 75: The method of any one of aspects 49-74, wherein a facial acne peel is administered by a clinician to the subject at the start of treatment in Week 1, in Week 3, in Week 5, in Week 7, in Week 9, in Week 11, in Week 13, or in Week 15, or any combination thereof if tolerated by the subject.

Aspect 76: The method of any one of aspects 49-75, wherein an acne hydrating treatment is administered by a clinician to the subject at the start of treatment, at Week 1, at Week 3, at Week 5, at Week 7, at Week 9, at Week 11, at Week 13, or at Week 15, or any combination thereof upon determining that an acne peel will not be well-tolerated by the subject at the time.

Aspect 77: The method of any one of the preceding aspects, wherein the subject achieves a significant clearance in acne compared to baseline by Week 10, by Week 12, by Week 14, or by Week 16.

Aspect 78: The method of any one of the preceding aspects, wherein the subject achieves a significant clearance in acne as defined by having an Investigator's Global Assessment (IGA) of acne severity score of 0 or 1, with at least a two-grade improvement from baseline.

Aspect 79: The method of any one of the preceding aspects, wherein the subject achieves a statistically significant clearance in acne as defined by having an IGA score of 0 or 1, with at least a two-grade improvement from baseline, by Week 14.

Aspect 80: The method of any one of the preceding aspects, wherein the subject achieves a statistically significant clearance in acne as defined by having an IGA score of 0 or 1, with at least a two-grade improvement from baseline, by Week 16.

Aspect 81: The method of any one of aspects 49-80, wherein the subject has mild noninflamed acne, and wherein the subject does not have inflamed acne.

Aspect 82: The method of any one of aspects 50-81, wherein the sunscreen is a broad-spectrum sunscreen.

Aspect 83: The method aspect 82, wherein the broad-spectrum sunscreen is a mineral-based sunscreen.

Aspect 84: The method of aspect 83, wherein the mineral-based sunscreen is rated to at least SPF 50.

Aspect 85: The method of aspect 82, wherein the broad-spectrum sunscreen is a chemical-based sunscreen.

Aspect 86: The method of any one of aspects 82-85, wherein the broad-spectrum sunscreen is a non-comedogenic sunscreen.

Aspect 87: The method of aspect 86, wherein the non-comedogenic sunscreen is rated to at least SPF 30 or to at least SPF 50.

Aspect 88: A system for clearing inflamed acne or combination acne in a subject in need thereof, the system comprising:
a facial cleanser, a toner, a serum, an OTC acne medication, a hydrator, a moisturizer, a sunscreen, and instructions, wherein the system is configured to perform the method steps of a customized skin care regimen comprising:
   (i) cleansing the face of the subject at least twice daily with the facial cleanser;
   (ii) optional applying of ice to the face of the subject at least twice daily following the cleansing;
   (iii) applying the toner to the skin of the subject following the cleansing in (i), or the optional application of the ice in (ii);
   (iv) applying of the serum to the face of the subject at least twice daily following the application the toner;
   (v) applying the OTC acne medication comprising an advanced topical acne clearing formulation comprising micronized benzoyl peroxide;
   (vi) optionally applying the moisturizer to the face of the subject;
   (vii) applying the sunscreen to the face of the subject daily in the AM; and in-clinic treatment comprising at least one facial acne peel or an acne hydrating treatment.

Aspect 89: The system of aspect 88, wherein the customized skin care regimen further comprises applying the hydrator to the face of the subject.

Aspect 90: The system of aspect 89, wherein the moisturizer is applied following the application of the hydrator.

Aspect 91: A system for clearing noninflamed acne in a subject in need thereof, the system comprising: a facial cleanser, a toner, a serum, an OTC acne medication, a hydrator, an optional moisturizer, a sunscreen, and instructions, wherein the system is configured to perform the method steps of a customized skin care regimen comprising:
(i) cleansing the face of the subject at least twice daily with the facial cleanser;
(ii) applying of the toner to the face of the subject at least twice daily following the cleansing;
(iii) applying of the serum to the face of the subject at least twice daily following the application the toner;
(iv) applying of the OTC acne medication to the face of the subject;
(v) optionally applying the moisturizer to the face of the subject;
(vi) applying the sunscreen to the face of the subject daily in the AM; and in-clinic treatment comprising at least one facial acne peel or an acne hydrating treatment.

Aspect 92: The system of aspect 91, wherein the OTC acne medication is an advanced topical acne clearing formulation comprising micronized benzoyl peroxide.

Aspect 92: A system for clearing mild noninflamed acne in a subject in need thereof, the system comprising: a facial cleanser, a toner, a serum, a hydrator, an optional water-based moisturizer, a sunscreen, and instructions, wherein the system is configured to perform the method steps of a customized skin care regimen comprising:
(i) cleansing the face of the subject at least twice daily with the facial cleanser;
(ii) applying of the toner to the face of the subject at least twice daily following the application of ice;
(iii) applying of the serum to the face of the subject at least twice daily following the application the toner;
(iv) optional applying of a hydrating gel to the face of the subject once or twice daily following the application the serum;
(v) optional applying of the water-based moisturizer to the face of the subject once or twice daily following either the application the serum or the application of the hydrating gel;
(vi) applying the sunscreen to the face of the subject daily in the AM; and in-clinic treatment comprising at least one facial acne peel or an acne hydrating treatment.

Aspect 93: The method of aspect 92, wherein the mild noninflamed acne in the subject is improved by at least about 2 weeks, about 3 weeks, about 4 weeks, about 5 weeks, about 6 weeks, about 7 weeks, about 8 weeks, about 9 weeks, or more than about 9 weeks after the start of the customized skin care regimen.

What is claimed is:

1. A method for clearing inflamed or combination acne in a subject in need thereof, the method comprising:
(i) assessing an extent of acne severity in the subject;
(ii) determining a skin type of the subject;
(iii) assigning a customizable skin care regimen to the subject, wherein the customizable skin care regimen is initiated by the subject and comprises:
a. cleansing the skin of the subject with a facial cleanser;
b. optionally applying ice to the skin of the subject following the cleansing;
c. applying a toner to the skin of the subject following the cleansing in a), or applying a toner to the skin of the subject following the optional application of the ice in b);
d. applying a serum to the skin of the subject following the application of the toner;
e. applying a topical bactericidal-based acne treatment formulation to the skin of the subject following the application of the serum, wherein the topical bactericidal-based acne treatment formulation is selected from:
i. a benzoyl peroxide-based acne treatment topical formulation;
ii. a salicylic acid-based acne treatment topical formulation; or
iii. a sulfur-based acne treatment topical formulation;
f. applying a hydrator, a moisturizer, or the hydrator and the moisturizer to the skin of the subject;
g. applying a non-comedogenic sunscreen to the skin of the subject daily in the AM; and
(iv) conducting an in-clinic assessment and treatment comprising at least one facial acne peel or an acne hydrating treatment, wherein the in-clinic assessment and treatment are each administered by a clinician.

2. The method of claim 1, wherein the in-clinic assessment and treatment begin on or around the same starting date as the customizable skin care regimen begins, wherein the in-clinic assessment and treatment are recurrently conducted by the clinician until the subject has achieved a significant extent of clearance of inflamed acne or combination acne, and wherein the subject has mild, moderate, or severe inflamed or combination acne at the time of a first in-clinic assessment.

3. The method of claim 2, wherein the in-clinic assessment and treatment are used to revise the customizable skin care regimen to: i) achieve favorable acne clearance and improved facial skin condition in the subject, ii) improve tolerance to skin care products selected as part of the customizable skin care regimen, or iii) achieve favorable acne clearance and improved facial skin condition in the subject and improve tolerance to skin care products selected as part of the customizable skin care regimen.

4. The method of claim 3, wherein an optional hydrator is applied to the skin of the subject.

5. The method of claim 3, wherein the subject is assigned an initial skin care routine for twice daily use at home, wherein the initial skin care routine is customized to the skin type of the subject, and wherein the skin type of the subject is (i) dry, (ii) mature, (iii) Fitzpatrick type 4, 5, or 6; (iv) normal, (v) oily and Fitzpatrick type 4, 5, or 6, (vi) oily, or (vii) oily in a subject that is pregnant or nursing (oily P/N).

6. The method of claim 5, wherein the skin type of the subject is (i) dry, (ii) mature, or (iii) Fitzpatrick type 4, 5, or 6; wherein the cleanser is an amino acid surfactant-based cleanser, wherein the toner is a hydrating toner, wherein the serum is a 5% solution of an alpha hydroxy acid-based serum or an 8% solution of an alpha hydroxy acid-based serum, and wherein the topical bactericidal-based acne treatment formulation comprises about 2.5% micronized benzoyl peroxide.

7. The method of claim 5, wherein the skin type of the subject is (iv) normal, wherein the cleanser is a surfactant-based gel cleanser, wherein the toner is a soothing facial toner, wherein the serum is a 5% solution of an alpha hydroxy acid-based serum or an 8% solution of an alpha hydroxy acid-based serum, and wherein the topical bactericidal-based acne treatment formulation comprises about 5% micronized benzoyl peroxide.

8. The method of claim 5, wherein the skin type of the subject is (v) oily and Fitzpatrick type 4, 5, or 6; wherein the cleanser is a surfactant-based gel cleanser, wherein the toner is a soothing facial toner, wherein the serum is a 5% solution of an alpha hydroxy acid-based serum or an 8% solution of an alpha hydroxy acid-based serum, and wherein the topical bactericidal-based acne treatment formulation comprises about 2.5% micronized benzoyl peroxide.

9. The method of claim 5, wherein the skin type of the subject is (vi) oily and wherein the toner is a salicylic-acid and vitamin C-based toner, or wherein the skin type of the subject is (vii) oily P/N and wherein the toner is a soothing facial toner; wherein the cleanser is a surfactant-based gel cleanser or a mandelic acid-based cleanser; wherein the serum is a 5% solution of an alpha hydroxy acid-based serum or an 8% solution of an alpha hydroxy acid-based serum; and wherein the topical bactericidal-based acne treatment formulation comprises about 5% micronized benzoyl peroxide.

10. The method of claim 5, wherein from Week 7 onward after the customizable skin care regimen has been initiated by the subject, a strength of the serum applied in (iii) d) is increased if a total inflamed acne count in the subject is less compared to a total inflamed acne count at the initiation of the customizable skin care regimen and a total noninflamed acne count is the same or increased compared to a total noninflamed acne count at the initiation of the customizable skin care regimen.

11. The method of claim 5, wherein from Week 7 onward after the customizable skin care regimen has been initiated by the subject, the serum in (iii) d) is changed to a retinaldehyde serum if a total inflamed acne count in the subject is zero and a total noninflamed acne count is the same or increased compared to a total noninflamed acne count at the initiation of the customizable skin care regimen.

12. The method of claim 5, wherein from Week 7 onward after the customizable skin care regimen has been initiated by the subject, a strength of the topical bactericidal-based acne treatment formulation applied in (iii) e) is increased if a total inflamed acne count in the subject is the same or increases compared to a total inflamed acne count at the initiation of the customizable skin care regimen.

13. The method of claim 1, wherein the subject achieves a significant clearance in acne as defined by having an Investigator's Global Assessment (IGA) of acne severity score of 0 or 1, with at least a two-grade improvement from baseline.

14. A system for clearing inflamed acne or combination acne in a subject in need thereof, the system comprising:
a facial cleanser, a toner, a serum, an over the counter (OTC) acne medication, a hydrator, an optional moisturizer, a sunscreen, and instructions, wherein the system is configured to perform the method steps of a customized skin care regimen comprising:
(i) cleansing the face of the subject at least twice daily with the facial cleanser;
(ii) optionally applying ice to the skin of the subject following the cleansing;
(iii) applying the toner to the face of the subject at least twice daily following the application of ice if ice has been applied in (ii), or following the cleansing in (i);
(iv) applying the serum to the face of the subject following the application of the toner;
(v) applying the OTC acne medication, wherein the OTC acne medication comprises an advanced topical acne clearing formulation comprising micronized benzoyl peroxide;
(vi) applying a hydrator, a moisturizer, or the hydrator and the moisturizer to the skin of the subject;
(vii) applying a non-comedogenic sunscreen to the skin of the subject daily in the AM; and
in-clinic treatment comprising at least one facial acne peel or an acne hydrating treatment.

15. A method for clearing noninflamed acne in a subject in need thereof, the method comprising:
(i) assessing an extent of acne severity in the subject;
(ii) determining a skin type of the subject;
(iii) assigning a customizable skin care regimen to the subject, wherein the customizable skin care regimen is initiated by the subject and comprises:
a. cleansing the skin of the subject with a facial cleanser;
b. applying a toner to the skin of the subject following the cleansing;
c. applying a serum to the skin of the subject following the application of the toner;
d. applying a topical bactericidal-based acne treatment formulation to the skin of the subject following the application of the serum, wherein the topical bactericidal-based acne treatment formulation is selected from:
i. a benzoyl peroxide-based acne treatment topical formulation;
ii. a salicylic acid-based acne treatment topical formulation; or
iii. a sulfur-based acne treatment topical formulation;
e. applying a moisturizer, a hydrator, or the moisturizer and the hydrator to the skin of the subject;
f. applying a non-comedogenic sunscreen to the skin of the subject daily in the AM; and
(iv) conducting an in-clinic assessment and treatment comprising at least one facial acne peel or an acne hydrating treatment, wherein the in-clinic assessment and treatment are each administered by a clinician.

16. The method of claim 15, wherein the in-clinic assessment and treatment begin on or around the same starting date as the customizable skin care regimen begins, wherein the in-clinic assessment and treatment are recurrently conducted by the clinician until the subject has achieved a significant extent of clearance of noninflamed acne, wherein the subject has moderate or severe noninflamed acne at the time of a first in-clinic assessment, and wherein the subject does not have inflamed acne at the time of the first in-clinic assessment.

17. The method of claim 15, wherein the in-clinic assessment and treatment are used to revise the customizable skin care regimen to: i) achieve favorable acne clearance; ii) improve facial skin condition in the subject; iii) improve tolerance to skin care products selected as part of the customizable skin care regimen, or any combination thereof.

18. The method of claim 15, wherein the moisturizer in e) is applied, and wherein an optional hydrator is applied to the skin of the subject and maintains skin of the subject in a healthy state of hydration so that excess oil is not produced.

19. The method of claim 15, wherein the subject is assigned an initial skin care routine for twice daily use at home, wherein the initial skin care routine is customized to the skin type of the subject, and wherein the skin type of the subject is (i) dry, (ii) mature, (iii) Fitzpatrick type 4, 5, or 6, (iv) normal, (v) oily and Fitzpatrick type 4, 5, or 6; (vi) oily, or (vii) oily in a subject that is pregnant or nursing (oily P/N).

20. The method of claim 19, wherein the skin type of the subject is: (i) dry, (ii) mature, or (iii) Fitzpatrick type 4, 5, or 6; wherein the cleanser is an amino acid surfactant-based cleanser or a sulfate-free gel cleanser; wherein the toner is a hydrating toner; wherein the serum is a 5% solution of an alpha hydroxy acid-based serum or an 8% solution of an alpha hydroxy acid-based serum; and wherein the topical bactericidal-based acne treatment formulation comprises about 2.5% micronized benzoyl peroxide.

21. The method of claim 19, wherein the skin type of the subject is: (iv) normal or (v) oily and Fitzpatrick type 4, 5, or 6 and wherein the cleanser is a surfactant-based gel cleanser; or wherein the skin type of the subject is (vi) oily, or (vii) oily P/N and wherein the cleanser is an surfactant-based gel cleanser or a surfactant-based exfoliating gel cleanser; wherein the toner is a soothing facial toner; wherein the serum is a 5% solution of an alpha hydroxy acid-based serum or an 8% solution of an alpha hydroxy acid-based serum; and wherein the topical bactericidal-based acne treatment formulation comprises about 2.5% micronized benzoyl peroxide or about 5% micronized benzoyl peroxide.

22. The method of claim 19, wherein from Week 7 onward after the customizable skin care regimen has been initiated by the subject, if a total noninflamed acne count in the subject increases compared to a previous assessment in the subject, application of a retinaldehyde serum to the subject is added to the customizable skin care regimen in each evening.

23. The method of claim 15, wherein the subject achieves a significant clearance in acne as defined by having an Investigator's Global Assessment (IGA) of acne severity score of 0 or 1, with at least a two-grade improvement from baseline.
